(19) United States
(12) Patent Application Publication
(10) Pub. No.: US 2019/0002613 A1
(43) Pub. Date: Jan. 3, 2019

(54) HYDROPHOBICALLY MODIFIED ALKALI-SWELLABLE EMULSION POLYMERS

(71) Applicant: Lubrizol Advanced Materials, Inc., Cleveland, OH (US)

(72) Inventors: Shui-Jen Raymond Hsu, Westlake, OH (US); Yi Yang, Bridgewater, NJ (US); Sinan Li, Hudson, OH (US); Qunhua Xu, Milltown, NJ (US)

(21) Appl. No.: 16/065,143

(22) PCT Filed: Dec. 19, 2016
(86) PCT No.: PCT/US16/67508

§ 371 Date: Jun. 22, 2018

Related U.S. Application Data
(60) Provisional application No. 62/387,587, filed on Dec. 23, 2015.

(51) Int. Cl. C08F 220/28; C08F 220/18; C08F 220/06; C08J 3/05; A61K 8/06; A61K 8/81; A61Q 5/02; A61Q 5/12; A61Q 19/10; A61Q 5/06; A61Q 1/02

(52) U.S. Cl. CPC C08F 220/28; A61K 2800/33; C08F 220/06; C08J 3/05; A61K 8/06; A61K 8/8152; A61K 8/8147; A61Q 5/02; A61Q 5/12; A61Q 19/10; A61Q 5/06; A61Q 1/02; C08F 2810/20; C08F 2800/20; C08F 2220/281; A61K 2800/30; A61K 2800/48; A61K 2800/26; C08F 220/18

(57) ABSTRACT

The present technology relates to hydrophobically modified alkali-swellable emulsion polymers that are useful as rheology modifiers. More specifically the present technology relates to hydrophobically modified alkali-swellable emulsion polymers containing residues of a polyunsaturated amphiphilic macromonomer. In one aspect the disclosed polymers are useful for thickening aqueous surfactant containing compositions, while providing the compositions in which they are included excellent rheology properties, clarity, and the ability to stably suspend insoluble and particulate materials over long periods of time.

### FIELD

The present technology relates to alkali-swellable emulsion polymers that are useful as rheology modifiers for aqueous systems. More specifically the present technology relates to hydrophobically modified alkali-swellable emulsion polymers containing residues of a polyunsaturated amphiphilic monomer. In one embodiment the disclosed polymers are useful for thickening aqueous surfactant containing compositions.

### BACKGROUND

Rheology modifiers are used as thickeners and structurants in a variety of industrial, consumer and pharmaceutical products. They affect product performance, aesthetics, application and suspension and the delivery of active chemical agents. It is standard practice to include rheology modifiers in personal care products in order to achieve optimum rheology properties. Various polymer types have been proposed for the purpose of increasing the rheology characteristics of personal care compositions, and are classified in several categories according to their chemical structure, physical form and on the mechanism by which they thicken.

Swellable acrylic emulsion polymers have long been employed in the art to thicken aqueous compositions. There are two major classes of swellable acrylic emulsion polymer thickeners: alkali-swellable emulsions (ASE) and hydrophobically modified alkali-swellable emulsions (HASE). An ASE thickener typically is a crosslinked copolymer that is prepared from ethylenically polymerizable monomers including a monomer containing an acid group (e.g., (meth)acrylic acid), a monomer containing a nonionic group (e.g., a non-water soluble lower alkyl ester of (meth)acrylic acid) and an ethylenically polyunsaturated monomer for crosslinking. A HASE thickener is a copolymer typically prepared from a monomer containing an acid group (e.g., (meth)acrylic acid), a monomer containing a nonionic group (e.g., a non-water soluble lower alkyl ester of (meth)acrylic acid) and an associative monomer containing hydrophobic groups (e.g., a hydrophobically modified polyoxyalkylene ester of (meth)acrylic acid).

The ASE polymers thicken aqueous systems by a hydrodynamic thickening mechanism. As supplied the majority of the acid groups on the polymer are in the protonated state. In this state the polymer molecules are tightly coiled imparting relatively little viscosity or suspension properties to the aqueous medium in which they are included. When neutralized with an inorganic or organic base the acid groups ionize causing the polymer to uncoil and extend due to charge repulsion of the ionized (anionic) carboxylate groups. In this hydrodynamic thickening mechanism the thickening and suspending effects of the neutralized polymers are due to the increased physical packing of the expanded polymer molecules (microgels) sometimes referred to as “space filling” or “volume exclusion”.

Unlike the ASE polymeric thickeners, HASE polymers contain pendant hydrophobic groups situated along the backbone. The hydrophobic groups are spaced from the polymer backbone via the polyalkylene oxide moieties. This polymeric thickener class functions by a dual thickening mechanism. Upon neutralization with an inorganic or organic base HASE polymers expand and swell as described for the ASE hydrodynamic thickening mechanism. In addition, the hydrophobic groups situated along the polymer chains interact with each another as well as with extrinsic hydrophobic components contained in the medium in which the polymer is included forming three-dimensional intramolecular and intermolecular hydrophobic associations or networks. These networks, combined with the hydrodynamic exclusion mechanism created by the expanded HASE chains, produces the desired thickening effect. The extrinsic hydrophobic components can be hydrophobic groups contained in surfactants, oils, long carbon chain esters, insoluble particles and the like.

While a rheology modifier may thicken or enhance the viscosity of a composition in which it is included, it does not necessarily provide desirable yield stress properties. A yield stress property is critical to achieving certain physical and aesthetic characteristics in a liquid medium, such as the indefinite suspension of particles, insoluble liquid droplets, or the stabilization of gas bubbles within the medium. Particles dispersed in a liquid medium will remain suspended if the yield stress (yield value) of the medium is sufficient to overcome the effect of gravity or buoyancy on those particles. Insoluble liquid droplets can be prevented from rising and coalescing and gas bubbles can be suspended and uniformly distributed in a liquid medium using yield value as a formulating tool. A yield stress polymer is used generally to adjust or modify the rheological properties of aqueous compositions. Such properties include, without limitation, viscosity improvement, flow rate improvement, stability to viscosity change over time, and the ability to suspend particles for indefinite periods of time.

It is known to covalently crosslink an ASE rheology modifying polymer to impart yield stress properties to the aqueous medium in which it is dispersed (Principles of Polymer Science and Technology in Cosmetics and Personal Care, Ch. 6, pp. 233-235; Marcel Dekker, Inc., 1999). U.S. Pat. No. 6,635,702 to Lubrizol Advanced Materials, Inc. discloses a crosslinked ASE polymer for use in aqueous surfactant containing compositions to thicken and stabilize products containing insoluble and particulate materials. The disclosed compositions are demonstrated to be stable and have an attractive visual appearance.

In contrast the HASE polymers generally rely on physical crosslinking formed from the associations of the hydrophobic moieties contained on the polymer backbone. As specified on page 239 of Principles of Polymer Science and Technology in Cosmetics and Personal Care, supra, “These interpolymeric associations create transient, non-covalent, interpolymeric cross-links.” The transient nature of these “crosslinks” is not conducive to the maintenance of stable yield value properties over extended periods of time. The stable long term suspension of insoluble and particulate materials is not achieved by conventional linear HASE polymers.

Exemplary HASE polymers are disclosed in U.S. Pat. No. 3,657,175; U.S. Pat. No. 4,384,096; U.S. Pat. No. 4,464,524; U.S. Pat. No. 4,801,671; U.S. Pat. No. 5,292,843, U.S. Pat. No. 5,874,495; U.S. Pat. No. 7,649,047; and U.S. Pat. No. 7,288,616. An extensive review of HASE polymers is found in Gregory D. Shay, Chapter 25, “Alkali-Swellable and Alkali-Soluble Thickener Technology A Review”, Polymers in Aqueous Media-Performance Through Association, Advances in Chemistry Series 223, J. Edward Glass (ed.), ACS, pp. 457-494, Division Polymeric Materials, Washington, D.C. (1989). The published literature discloses that HASE polymers can be optionally crosslinked.

While HASE polymers have been crosslinked, the crosslinking agents are conventional crosslinking monomers containing at least two ethylenically polymerizable unsaturated moieties. These are relatively low molecular weight molecules (typically less than 300 Daltons). Exemplary crosslinkers employed in the emulsion polymerization of acrylic based monomers are polyvinyl aromatic monomers (e.g., divinylbenzene, divinyl naphthalene, and trivinylbenzene); polyunsaturated alicyclic monomers (e.g., 1,2,4-trivinylcyclohexane; di-functional esters of phthalic acid (e.g., diallyl phthalate); polyalkenyl ethers (e.g, triallyl pentaerythritol, diallyl pentaerythritol, diallyl sucrose, octaallyl sucrose, and trimethylolpropane diallyl ether); polyunsaturated esters of polyalcohols or polyacids (e.g., 1,6-hexanediol di(meth)acrylate, tetramethylene tri(meth)acrylate, allyl (meth)acrylate, diallyl itaconate, diallyl fumarate, diallyl maleate, trimethylolpropane tri(meth)acrylate, trimethylolpropane di(meth)acrylate, and polyethylene glycol di(meth)acrylate); alkylene bisacrylamides (e.g, methylene bisacrylamide and propylene bisacrylamide); hydroxy and carboxy derivatives of methylene bis-acrylamide (e.g., N,N′-bismethylol methylene bisacrylamide); polyethyleneglycol di(meth)acrylates (e.g., ethyleneglycol di(meth)acrylate, diethyleneglycol di(meth)acrylate, and triethyleneglycol di(meth)acrylate.

A commercially available crosslinked HASE polymer is available under the INCI Name: Acrylates/Steareth-20 Methacrylate Crosspolymer and is identified under INCI Monograph ID 19820 to be a copolymer of stereth-20 methacrylate and one or more monomers consisting of methacrylic acid or some of their simple esters crosslinked with an allyl ether of pentaerythritol or an allyl ether of trimethylolpropane.

As previously discussed, hydrophobically modified crosslinked copolymers are viscosity building agents that increase the viscosity of compositions in which they are dissolved or dispersed. As increasing amounts of viscosity builder are added to a cleansing or cleaning there is a corresponding increase in the viscosity of the composition. It is well known in the personal care, household care and industrial and institutional care formulation art that a liquid cleanser or cleaner should have an ideal viscosity. Indeed, viscosity allows for a controlled handling and dispensing of the product during use as compared to a thinner product. In personal care cleansing applications, a thick, rich shampoo or body cleanser is appealing to consumers from a sensory perspective. In household care applications, viscosity permits a better efficacy of the product when applied to non-horizontal surfaces such as toilet bowls, sinks, shower stalls, bath tubs, and the like. In addition, cleansing and cleaning products are expected to be easy to use. In other words, the shear thinning profile of the liquid composition should exhibit high viscosity at low shear conditions and lower viscosity at high shear conditions to aid in the application and removal of the product from the substrate to be cleaned.

However, there are some drawbacks associated with increasing the viscosity of a product beyond its ideal viscosity. Very high viscous products are typically difficult to apply and rinse away, especially if the shear thinning profile of the viscosity building agent is deficient. High viscosities can also adversely affect packaging, dispensing, dissolution, and the foaming and sensory properties of the product. Accordingly, there is a need for HASE polymer that does not significantly change the ideal viscosity profile of a surfactant containing composition, is able to stably suspend insoluble and particulate materials for extended periods of time, and has good clarity properties.

Although the foregoing disclosures and their commercial embodiments provide rheology modifying properties, there still is a need for improvements in the thickening and suspension profile as well as in suspension clarity of aqueous surfactant containing compositions thickened with HASE polymers.

### SUMMARY OF THE DISCLOSED TECHNOLOGY

The present technology relates to hydrophobically modified alkali-swellable emulsion polymers referred to herein as HASE. The HASE polymers of the present technology are the polymerization product of a monomer mixture comprising (A) at least one acidic vinyl monomer; (B) at least one nonionic vinyl monomer; (C) at least one alkoxylated associative monomer having a hydrophobic end group; (D) at least one polyunsaturated amphiphilic macromonomer; and optionally one or more of, (E) at least one semihydrophobic monomer; (F) at least one crosslinking monomer, and mixtures of (E) and (F) thereof.

The rheology modifier is a copolymer represented by formula (I) below.

AₐB_{b}C_{c}D_{d}EₑF_{f} (I)

wherein (A) is a repeating unit of at least one acidic vinyl monomer residue; (B) is a repeating unit of at least one nonionic vinyl monomer residue; (C) is a repeating unit of at least one alkoxylated associative monomer residue; (D) is a repeating unit of at least one amphiphilic polyunsaturated macromonomer residue; E is a repeating unit of at least one semihydrophobic monomer residue; and (F) is a polyunsaturated crosslinking monomer residue; and wherein a, b, c, d, e and f represent the percentage by weight of each monomer repeating unit contained within the copolymer, and the sum of a+b+c+d+e+f is 100 weight percent.

In one aspect the polymerizable monomer mixture used to prepare the HASE polymers of the disclosed technology contains a chain transfer agent (G)_{g} wherein g represents the weight percent of the chain transfer agent present in the polymerizable monomer mixture, and the sum of a+b+c+d+e+f+g is 100 weight percent of the monomer mixture.

Monomer residues A, B, C, D, E and F are covalently linked to one another and can be arranged in random, block and branched architecture.

The HASE polymers of the present technology provide compositions having aesthetically pleasing rheological properties ranging from pourable liquids to non-pourable gels, as well as non-runny, yet flowable, compositions, without requiring additional or auxiliary rheology modifiers. The disclosed polymers can also suspend abrasives, pigments, particulates, water insoluble materials, such as encapsulated oil beads, liposomes, capsules, silicones, gaseous bubbles, and the like.

Advantageously, the HASE polymers of the disclosed technology can be employed, without limitation, in personal care products, health care products, household care products, non-household, institutional and industrial care products, and the like and in industrial chemical processes and applications as, for example, rheology modifiers, film formers, thickeners, emulsifiers, stabilizers, solubilizers, suspending agents, and pigment grinding additives. The disclosed HASE polymers are particularly useful as thickeners in personal care compositions, textile treatment compositions for finishing, coating and printing applications, and in industrial paints and coatings.

In particular, the HASE polymers of the disclosed technology are able to provide surfactant containing compositions over a wide concentration range with an ideal viscosity, long term suspension stability and clarity.

### BRIEF DESCRIPTION OF THE DRAWINGS

FIG. 1 illustrates an exemplary plot of the elastic (G′) and viscous moduli (G″) as a function of increasing oscillatory stress amplitude (Pa) for a polymer capable of providing a yield stress property to a surfactant containing formulation. The plot shows the crossover point of G′ and G″ corresponding to the yield stress value of the formulation.

### DESCRIPTION OF EXEMPLARY EMBODIMENTS

Exemplary embodiments in accordance with the disclosed technology will be described. Various modifications, adaptations or variations of the exemplary embodiments described herein may become apparent to those skilled in the art as such are disclosed. It will be understood that all such modifications, adaptations or variations that rely upon the teachings of the disclosed technology, and through which these teachings have advanced the art, are considered to be within the scope and spirit of the presently disclosed technology.

The compositions, polymers and methods of the disclosed technology may suitably comprise, consist of, or consist essentially of the components, elements, steps, and process delineations described herein. The technology illustratively disclosed herein suitably may be practiced in the absence of any element which is not specifically disclosed herein.

Except as otherwise noted, the articles “a”, “an”, and “the” mean one or more.

Unless otherwise stated, all percentages, parts, and ratios expressed herein are based upon weight of the total compositions of the disclosed technology.

When referring to a specified monomer(s) that is incorporated into a polymer of the disclosed technology, it will be recognized that the monomer(s) is incorporated into the polymer backbone as a unit(s) derived from the specified monomer(s) (e.g., monomer repeating unit or monomer residue).

As used herein, the term “amphiphilic” means that the constituent material has distinct hydrophilic and hydrophobic portions. “Hydrophilic” typically means a portion that interacts intramolecularly with water and other polar molecules. “Hydrophobic” typically means a portion that interacts preferentially with oils, fats or other non-polar molecules or components rather than aqueous media.

The prefix “(meth)acryl” includes “acryl” as well as “methacryl”. For example, the term (meth)acrylic includes both acrylic and methacrylic, and the term (meth)acrylate includes acrylate as well as methacrylate. By way of further example, the term “(meth)acrylamide” includes both acrylamide and methacrylamide.

Here, as well as elsewhere in the specification and claims, individual numerical values (including carbon atom numerical values), or limits, can be combined to form additional non-disclosed and/or non-stated ranges.

While overlapping weight ranges for the various compounds, components and ingredients that are contained in the polymers, compositions and formulations of the disclosed technology have been expressed for selected embodiments and aspects of the technology, it should be readily apparent that the specific amount of each component in the disclosed polymers, compositions and formulations will be selected from its disclosed range such that the amount of each component is adjusted so that the sum of all components in the polymer, composition or formulation will total 100 weight percent. The amounts employed will vary with the purpose and character of the desired product and can be readily determined by one skilled in the art.

The headings provided herein serve to illustrate, but not to limit the disclosed technology in any way or manner.

Acidic Vinyl Monomer (A)

Acidic vinyl monomers suitable for use in the present technology are acidic, polymerizable, ethylenically unsaturated monomers containing at least one carboxylic acid group, sulfonic acid group, or a phosphonic acid group to provide an acidic or anionic functional site. These acid groups can be derived from monoacids or diacids, anhydrides of dicarboxylic acids, monoesters of diacids, and their salts.

Suitable acidic vinyl carboxylic acid monomers include, but are not limited to, acrylic acid, methacrylic acid, itaconic acid, citraconic acid, maleic acid, fumaric acid, crotonic acid, aconitic acid, and their salts. Alkyl (C₁-C₁₈) monoesters of maleic, fumaric, itaconic, aconitic acid, and their salts, such as, for example, methyl hydrogen maleate, monoisopropyl maleate, butyl hydrogen fumarate can be utilized as acidic vinyl monomers. Anhydrides of dicarboxylic acids, such as, for example, maleic anhydride, itaconic anhydride, citraconic anhydride, and their salts also can be utilized as acidic vinyl monomers. Such anhydrides generally hydrolyze to the corresponding diacids upon prolonged exposure to water, or at elevated pH.

Suitable sulfonic acid group containing monomers include, but are not limited to, vinyl sulfonic acid, 2-sulfoethyl methacrylate, styrene sulfonic acid, 2-acrylamido-2-methylpropane sulfonic acid (AMPS™ monomer), allyloxybenzene sulfonic acid, and the like.

Non-limiting examples of suitable phosphonic acid group-containing monomers include vinyl phosphonic acid, allyl phosphonic acid, 3-acrylamidopropyl phosphonic acid, and the like.

Suitable salts of the acidic vinyl monomers include, without limitation, alkali metal salts, such as sodium, potassium and lithium salts; alkaline earth metal salts, such as calcium and magnesium salts; ammonium salts; and alkyl substituted ammonium salts, such as salts of 2-amino-2-methyl-1-propanol (AMP), ethanolamine, diethanolamine, triethanolamine, triethylamine, and the like.

The acidic vinyl monomers and/or the salts thereof can be utilized individually or in mixtures of two or more in the monomer mixture for preparing the disclosed polymers. The acidic vinyl monomer comprises from about 5 to about 75 weight percent of the total monomer mixture in one aspect, from about 10 to about 65 weight percent in another aspect, and from about 25 to about 60 weight percent in a further aspect, and from about 30 to about 45 in a still further aspect, based on the total monomer weight.

Nonionic Vinyl Monomer (B)

The nonionic vinyl monomers suitable for use in the disclosed technology are copolymerizable, nonionic, ethylenically unsaturated monomers. By nonionic is meant that the monomer (or monomer repeating unit) does not contain a positive or negative charge and does not ionize in an aqueous solution when exposed to an acidic or an alkaline pH. The nonionic vinyl monomer can be water soluble or water insoluble. In one aspect of the disclosed technology, the nonionic vinyl monomer is at least one compound selected from formula (I), at least one compound selected from formula (II), and mixtures of compounds selected from formula (I) and formula (II):

CH₂═C(X)Z (I)

CH₂═CH—OC(O)R (II)

wherein, in each of formulas (I) and (II), X is H or methyl; and Z is —C(O)OR¹, —C(O)NH₂, —C(O)NHR¹, —C(O)N(R¹)₂, —C₆H₄R¹, —C₆H₄OR¹, —C₆H₄Cl, —CN, —NHC(O)CH₃, —NHC(O)H, N-(2-pyrrolidonyl), N-caprolactamyl, —C(O)NHC(CH₃)₃, —C(O)NHCH₂CH₂—N-ethyleneurea, —SiR₃, —C(O)O(CH₂)ₓSiR₃, —C(O)NH(CH₂)ₓSiR₃, or —(CH₂)ₓSiR₃; x is an integer ranging from about 1 to about 6; each R is independently linear and branched C₁-C₁₈ alkyl; each R¹ is independently linear and branched C₁-C₃₀ alkyl, hydroxy substituted linear and branched C₂-C₃₀ alkyl, or halogen substituted linear and branched C₁-C₃₀ alkyl.

Non-limiting examples of suitable water insoluble, nonionic vinyl monomers include C₁-C₃₀ alkyl (meth)acrylates, such as methyl (meth)acrylate, ethyl (meth)acrylate, propyl (meth)acrylate, sec-butyl (meth)acrylate, iso-butyl (meth)acrylate, tert-butyl (meth)acrylate), hexyl (meth)acrylate, heptyl (meth)acrylate, octyl (meth)acrylate, 2-ethylhexyl (meth)acrylate, decyl (meth)acrylate, isodecyl (meth)acrylate, lauryl (meth)acrylate, tetradecyl (meth)acrylate, hexadecyl (meth)acrylate, stearyl (meth)acrylate, behenyl (meth)acrylate, and mixtures thereof; C₁-C₃₀ alkyl (meth)acrylamides; styrene; substituted styrenes, such as vinyl toluene (e.g., 2-methyl styrene), butyl styrene, isopropyl styrene, p-chloro styrene, and the like; vinyl esters, such as vinyl acetate, vinyl butyrate, vinyl caprolate, vinyl pivalate, vinyl neodecanoate, and the like; unsaturated nitriles, such as methacrylonitrile, acrylonitrile, and the like; and unsaturated silanes, such as trimethylvinylsilane, dimethylethylvinylsilane, allyldimethylphenylsilane, allytrimethylsilane, 3-acrylamidopropyltrimethylsilane, 3-trimethylsilylpropyl methacrylate, and mixtures thereof.

Non-limiting examples of suitable water soluble nonionic vinyl monomers are C₂-C₆ hydroxyalkyl (meth)acrylates (e.g., 2-hydroxyethyl(meth)acrylate, 3-hydroxypropyl(meth)acrylate, and 4-hydroxybutyl(meth)acrylate); glycerol mono(meth)acrylate; tris(hydroxymethyl)ethane mono(meth)acrylate; pentaerythritol mono(meth)acrylate; N-hydroxymethyl (meth)acrylamide; 2-hydroxyethyl (meth)acrylamide; 3-hydroxypropyl (meth)acrylamide; (meth)acrylamide; N-vinyl caprolactam; N-vinyl pyrrolidone; methacrylamidoethyl-N-ethyleneurea (e.g., CH₂═C(CH₃)C(O)NHCH₂CH₂—N-ethyleneurea), C₁-C₄ alkoxy-substituted (meth)acrylates and (meth)acrylamides, such as methoxyethyl (meth)acrylate, 2-(2-ethoxyethoxy)ethyl (meth)acrylate, and mixtures thereof.

The nonionic vinyl monomer comprises from about 10 to about 90 weight percent of the total monomer mixture in one aspect, from about 25 to about 75 weight percent in another aspect, and from about 30 to about 60 weight percent in a further aspect, based on a total monomer weight basis.

Alkoxylated Associative Monomer (C)

The alkoxylated associative monomer of the disclosed technology has an ethylenically unsaturated end group portion (i) for addition polymerization with the other monomers of the disclosed technology; a polyoxyalkylene mid-section portion (ii) for imparting selective hydrophilic and/or hydrophobic properties to the product polymer, and a hydrophobic end group portion (iii) for providing selective hydrophobic properties to the polymer.

The portion (i) supplying the ethylenically unsaturated end group can be a residue derived from an α,β-ethylenically unsaturated monocarboxylic acid. Alternatively, portion (i) of the associative monomer can be a residue derived from an allyl ether or vinyl ether; a nonionic vinyl substituted urethane monomer, such as disclosed in U.S. Reissue Pat. No. 33,156 or U.S. Pat. No. 5,294,692; or a vinyl-substituted urea reaction product, such as disclosed in U.S. Pat. No. 5,011,978; the relevant disclosures of each are incorporated herein by reference.

The mid-section portion (ii) is a polyoxyalkylene segment of about 2 to about 150 in one aspect, from about 5 to about 120 in another aspect, from about 10 to about 60 in a further aspect, and from about 15 to 30 in a still further aspect of repeating C₂-C₄ alkylene oxide units. The mid-section portion (ii) includes polyoxyethylene, polyoxypropylene, and polyoxybutylene segments, and combinations thereof comprising from about 2 to about 150 in one aspect, from about 5 to about 120 in another aspect, from about 10 to about 60 in a further aspect, and from about 15 to about 30 in a still further aspect of ethylene oxide units, propylene oxide units, butylene oxide units, and mixtures thereof. When the polyalkylene oxide units are selected from two or more different alkylene oxide unit types, they can be arranged in random or block architecture.

The hydrophobic end group portion (iii) of the alkoxylated associative monomer is a hydrocarbon moiety belonging to one of the following hydrocarbon classes: a C₈-C₃₀ linear alkyl, a C₈-C₃₀ branched alkyl, a C₂-C₃₀ alkyl-substituted phenyl, aryl-substituted C₂-C₃₀ alkyl groups, a C₇-C₃₀ saturated or unsaturated carbocyclic alkyl group. The saturated or unsaturated carbocyclic moiety can be a C₁-C₅ alkyl substituted or unsubstituted monocyclic or bicyclic moiety. In one aspect the bicyclic moiety is selected from bicycloheptyl or bicycloheptenyl. In another aspect the bicycloheptenyl moiety is disubstituted with the alkyl substituent(s). In a further aspect the bicycloheptenyl moiety is disubstituted with methyl on the same carbon atom.

Non-limiting examples of suitable hydrophobic end group portions (iii) of the alkoxylated associative monomers are linear or branched alkyl groups having about 8 to about 30 carbon atoms, such as capryl (C₈), iso-octyl (branched C₈), decyl (C₁₀), lauryl (C₁₂), myristyl (C₁₄), cetyl (C₁₆), cetearyl (C₁₆-C₁₈), stearyl (C₁₈), isostearyl (branched C₁₈), arachidyl (C₂₀), behenyl (C₂₂), lignoceryl (C₂₄), cerotyl (C₂₆), montanyl (C₂₈), melissyl (C₃₀), and the like.

Examples of linear and branched alkyl groups having about 8 to about 30 carbon atoms that are derived from a natural source include, without being limited thereto, alkyl groups derived from hydrogenated peanut oil, soybean oil and canola oil (all predominately C₁₈), hydrogenated tallow oil (C₁₆-C₁₈), and the like; and hydrogenated C₁₀-C₃₀ terpenols, such as hydrogenated geraniol (branched C₁₀), hydrogenated farnesol (branched C₁₅), hydrogenated phytol (branched C₂₀), and the like.

Non-limiting examples of suitable C₂-C₃₀ alkyl substituted phenyl groups include octylphenyl, nonylphenyl, decylphenyl, dodecylphenyl, hexadecylphenyl, octadecylphenyl, isooctylphenyl, sec-butylphenyl, and the like.

Exemplary aryl-substituted C₂-C₄₀ alkyl groups include, without limitation, styryl (e.g., 2-phenylethyl), distyryl (e.g., 2,4-diphenylbutyl), tristyryl (e.g., 2,4,6-triphenylhexyl), 4-phenylbutyl, 2-methyl-2-phenylethyl, tristyrylphenolyl, and the like.

Suitable C₇-C₃₀ carbocyclic groups include, without limitation, groups derived from sterols from animal sources, such as cholesterol, lanosterol, 7-dehydrocholesterol, and the like; from vegetable sources, such as phytosterol, stigmasterol, campesterol, and the like; and from yeast sources, such as ergosterol, mycosterol, and the like. Other carbocyclic alkyl hydrophobic end groups useful in the disclosed technology include, without limitation, cyclooctyl, cyclododecyl, adamantyl, decahydronaphthyl, and groups derived from natural carbocyclic materials, such as pinene, hydrogenated retinol, camphor, isobornyl alcohol, norbornyl alcohol, nopol and the like.

Useful alkoxylated associative monomers can be prepared by any method known in the art. See, for example, U.S. Pat. No. 4,421,902 to Chang et al.; U.S. Pat. No. 4,384,096 to Sonnabend; U.S. Pat. No. 4,514,552 to Shay et al.; U.S. Pat. No. 4,600,761 to Ruffner et al.; U.S. Pat. No. 4,616,074 to Ruffner; U.S. Pat. No. 5,294,692 to Barron et al.; U.S. Pat. No. 5,292,843 to Jenkins et al.; U.S. Pat. No. 5,770,760 to Robinson; U.S. Pat. No. 5,412,142 to Wilkerson, III et al.; and U.S. Pat. No. 7,772,421, to Yang et al., the pertinent disclosures of which are incorporated herein by reference.

In one aspect, exemplary alkoxylated associative monomers include those represented by formulas (III) and (liA) as follows:

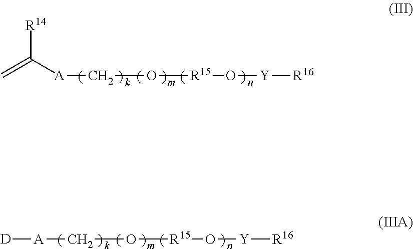

wherein R¹⁴ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; D represents a vinyl or an allyl moiety; (R¹⁵—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or 04H₈, and combinations thereof; and n is an integer in the range of about 2 to about 150 in one aspect, from about 10 to about 120 in another aspect, and from about 15 to about 60 in a further aspect; Y is —R¹⁵O—, —R¹⁵NH—, —C(O)—, —C(O)NH—, —R¹⁵NHC(O)NH—, —C(O)NHC(O)—, or a divalent alkylene radical containing 1 to 5 carbon atoms, e.g., methylene, ethylene, propylene, butylene, pentylene; R¹⁶ is a substituted or unsubstituted alkyl selected from a C₈-C₃₀ linear alkyl, a C₈-C₃₀ branched alkyl, a C₇-C₃₀ carbocyclic, a C₂-C₃₀ alkyl-substituted phenyl, an araalkyl substituted phenyl, and an aryl-substituted C₂-C₃₀ alkyl; wherein the R¹⁶ alkyl group, aryl group, phenyl group, or carbocyclic group optionally comprises one or more substituents selected from the group consisting of a methyl group, a hydroxyl group, an alkoxyl group, benzyl group phenylethyl group, and a halogen group. In one aspect, Y is ethylene and R¹⁶ is

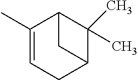

In one aspect, the hydrophobically modified alkoxylated associative monomer is an alkoxylated (meth)acrylate having a hydrophobic group containing 8 to 30 carbon atoms represented by the following Formula (IIIB) as follows:

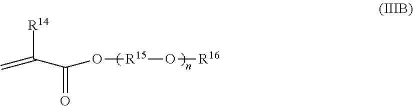

wherein R¹⁴ is hydrogen or methyl; R¹⁵ is a divalent alkylene moiety independently selected from C₂H₄, C₃H₆, and C₄H₈, and n represents an integer ranging from about 2 to about 150 in one aspect, from about 5 to about 120 in another aspect, from about 10 to about 60 in a further aspect, and from about 15 to about 30 in a still further aspect, (R¹⁵—O) can be arranged in a random or a block configuration; R¹⁶ is a substituted or unsubstituted alkyl selected from a C₈-C₃₀ linear alkyl, a C₈-C₃₀ branched alkyl, an alkyl substituted and unsubstituted C₇-C₃₀ carbocyclic alkyl, a C₂-C₃₀ alkyl-substituted phenyl, and an aryl-substituted C₂-C₃₀ alkyl.

Representative monomers under formula (IIIB) include lauryl polyethoxylated (meth)acrylate (LEM), cetyl polyethoxylated (meth)acrylate (CEM), cetearyl polyethoxylated (meth)acrylate (CSEM), stearyl polyethoxylated (meth)acrylate, arachidyl polyethoxylated (meth)acrylate, behenyl polyethoxylated (meth)acrylate (BEM), cerotyl polyethoxylated (meth)acrylate, montanyl polyethoxylated (meth)acrylate, melissyl polyethoxylated (meth)acrylate, phenyl polyethoxylated (meth)acrylate, nonylphenyl polyethoxylated (meth)acrylate, w-tristyrylphenyl polyoxyethylene (meth)acrylate, where the polyethoxylated portion of the monomer contains about 2 to about 150 ethylene oxide units in one aspect, from about 5 to about 120 in another aspect, from about 10 to about 60 in a further aspect and from about 15 to about 30 in a still further aspect; octyloxy polyethyleneglycol (8) polypropyleneglycol (6) (meth)acrylate, phenoxy polyethylene glycol (6) polypropylene glycol (6) (meth)acrylate, and nonylphenoxy polyethylene glycol polypropylene glycol (meth)acrylate.

The alkoxylated associative monomer comprises from about 0.1 to about 25 weight percent of the total monomer mixture in one aspect, from about 0.5 to about 20 weight percent in another aspect, from about 1 to about 15 weight percent in a further aspect, and from about 5 to about 10 weight percent in a still further aspect based on a total monomer weight basis.

Amphiphilic Polyunsaturated Macromonomer (D)

The amphiphilic polyunsaturated macromonomer contains a hydrophobic moiety and a hydrophilic moiety. The hydrophobic moiety provides solubility in oils, and the hydrophilic moiety provides water solubility. The amphiphilic nature of the macromonomer conveys surfactant-like properties to the polymer in which it is included.

The amphiphilic polyunsaturated macromonomers have a molecular weight of at least 500 daltons in one aspect, 500 to 60,000 daltons in another aspect, 1,000 to 50,000 daltons in still another aspect, 1500 to 30,000 daltons in a further aspect, and 2,000 to 25,000 daltons in a still further aspect.

In one aspect, exemplary amphiphilic polyunsaturated macromonomers suitable for use with the present technology can include, but not be limited to, compounds such as those disclosed in US 2013/0047892 (published Feb. 28, 2013 to Palmer, Jr. et al.), represented by the following formulas:

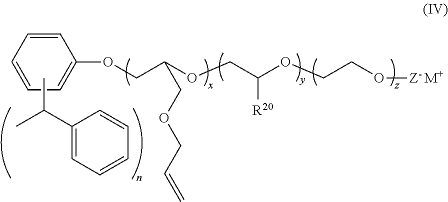

where R²⁰ is CH₃, CH₂CH₃, C₆H₅, or C₁₄H₂₉; n is1, 2, or 3; x is 2-10, y is 0-200, z is 4-200, more preferably from about 5 to 60, and most preferably from about 5 to 40; Z can be either SO₃⁻ or PO₃²⁻, and M⁺ is N⁺, K⁺, NH₄⁺, or an alkanolamine such as, for example, monoethanolamine, diethanolamine, and triethanolamine;

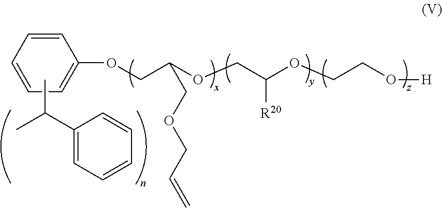

where R²⁰ is CH₃, CH₂CH₃, C₆H₅, or C₁₄H₂₉; n is 1, 2, 3; x is 2-10, y is 0-200, z is 4-200 in one aspect, from about 5 to 60 in another aspect, and from about 5 to 40 in a further aspect;

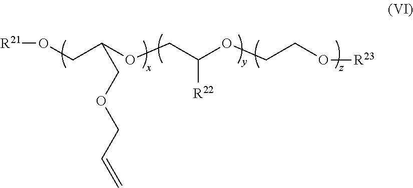

where R²¹ is a C₈-C₃₀ alkyl, alkaryl, alkenyl, or cycloalkyl group in one aspect, and a C₁₀-C₂₄ alkyl, aryl, alkylaryl, and aralkylaryl group in another aspect; R²² is CH₃, CH₂CH₃, C₆H₅, or C₁₄H₂₉; x is 2-100 in one aspect, and 2-10 in another aspect, y is 0-200 in one aspect, and from 0 or 1-50 in another aspect, z is 4-200 in one aspect, from about 5 to 60 in another aspect, and from about 5-40 in a further aspect; and R²³ is H or Z⁻M⁺, where Z can be either SO₃⁻ or PO₃²⁻, and M⁺ is Na⁺, K⁺, NH₄⁺, or an alkanolamine such as, for example, monoethanolamine, diethanolamine, and triethanolamine.

In one aspect, the polyunsaturated macromonomer is selected from the compounds represented by formulas (VI), (VII) or (VIII).

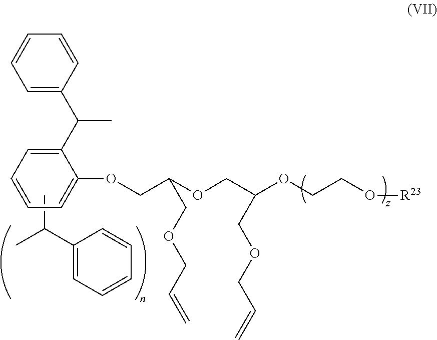

where n is 1 or 2; z is 4-40 in one aspect, 5-38 in another aspect, and 10-20 in a further aspect; and R²³ is H, SO₃⁻M⁺ or PO₃⁻M⁺, and M is selected from Na, K, and NH₄.

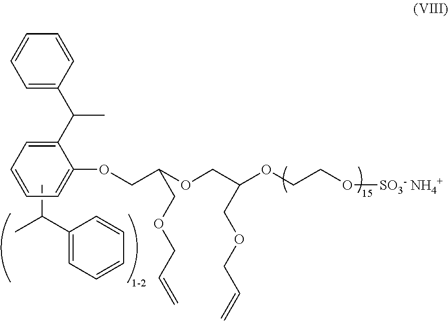

In one embodiment, the amphiphilic polyunsaturated macromonomer can be used in an amount ranging from about 0.01 to about 20 weight percent in one aspect, from about 0.5 to about 10 weight percent in another aspect, from about 0.75 to about 7 weight percent in still another aspect, from about 1 to about 5 weight percent in a further aspect, and from about 1.5 to about 3 weight percent in a still further aspect, based on the total weight of the monounsaturated monomers utilized to prepare the HASE polymers of the disclosed technology. Stated another way, the amount of amphiphilic macromonomer can be calculated on the basis of parts by wt. (100% active material) per 100 parts by wt. (100% active material) of total monounsaturated monomers utilized to prepare the HASE polymer of the disclosed technology.

In one embodiment, the amphiphilic polyunsaturated macromonomer contains an average of about 1.5 to about 2 unsaturated moieties in the molecule.

Alkoxylated Semi-Hydrophobic Monomer (E)

The alkoxylated semi-hydrophobic monomers of the disclosed technology are structurally similar to the alkoxylated associative monomer described above, but have a substantially non-hydrophobic end group portion. They may optionally be included in the polymer. The alkoxylated semi-hydrophobic monomer contains an ethylenically unsaturated end group portion (i) for addition polymerization with the other monomers of the disclosed technology; a polyoxyalkylene mid-section portion (ii) for imparting selective hydrophilic and/or hydrophobic properties to the product polymer and a semi-hydrophobic end group portion (iii). The unsaturated end group portion (i) supplying the vinyl or other ethylenically unsaturated end group for addition polymerization is preferably derived from an α,β-ethylenically unsaturated mono carboxylic acid. Alternatively, the end group portion (i) can be derived from an allyl ether residue, a vinyl ether residue or a residue of a nonionic urethane monomer.

The polyoxyalkylene mid-section portion (ii) includes polyoxyethylene, polyoxypropylene, and polyoxybutylene segments, and combinations thereof comprising from about 2 to about 150 in one aspect, from about 5 to about 120 in another aspect, from about 10 to about 60 in a further aspect, and from about 15 to about 30 in a still further aspect of ethylene oxide units, propylene oxide units, butylene oxide units, and mixtures thereof. When the polyalkylene oxide units are selected from two or more different alkylene oxide unit types, they can be arranged in random or block architecture.

In one aspect, the alkoxylated semi-hydrophobic monomer can be represented by formulas (IX) and (X):

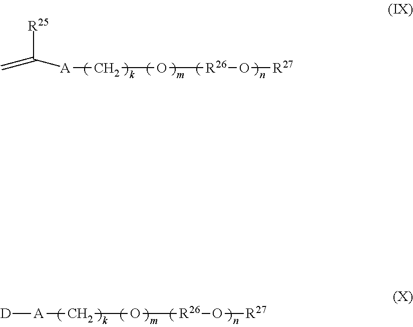

wherein R²⁵ is hydrogen or methyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent arylene (e.g., phenylene); E is H or methyl; z is 0 or 1; k is an integer ranging from about 0 to about 30, and m is 0 or 1, with the proviso that when k is 0, m is 0, and when k is in the range of 1 to about 30, m is 1; (R²⁶—O)ₙ is a polyoxyalkylene moiety, which can be a homopolymer, a random copolymer, or a block copolymer of C₂-C₄ oxyalkylene units, R¹⁵ is a divalent alkylene moiety selected from C₂H₄, C₃H₆, or C₄H₈, and combinations thereof; and n is an integer in the range of about 2 to about 150 in one aspect, from about 5 to about 120 in another aspect, and from about 10 to about 60, and from about 15 to about 30 in a still further aspect in a further aspect; R²⁷ is selected from hydrogen and a linear or branched C₁-C₄ alkyl group (e.g., methyl, ethyl, propyl, iso-propyl, butyl, iso-butyl, and tert-butyl); and D represents a vinyl or an allyl moiety.

In one aspect, the alkoxylated semi-hydrophobic monomer under formula (IX) can be represented by the following formulas:

CH₂═C(R²⁵)C(O)O—(C₂H₄O)ₐ(C₃H₆O)_{b}—H IXA

CH₂═C(R²⁵)C(O)O—(C₂H₄O)ₐ(C₃H₆O)_{b}—CH₃ IXB

wherein R²⁵ is hydrogen or methyl, and “a” is an integer ranging from 0 or 2 to about 120 in one aspect, from about 5 to about 45 in another aspect, and from about 10 to about 25 in a further aspect, and “b” is an integer ranging from about 0 or 2 to about 120 in one aspect, from about 5 to about 45 in another aspect, and from about 10 to about 25 in a further aspect, subject to the proviso that “a” and “b” cannot be 0 at the same time.

Examples of alkoxylated semi-hydrophobic monomers under formula IXA include polyethyleneglycol methacrylate available under the product names Blemmer® PE-90 (R²⁵=methyl, a=2, b=0), PE-200 (R²⁵=methyl, a=4.5, b=0), and PE-350 (R²⁵=methyl a=8, b=0,); polypropylene glycol methacrylate available under the product names Blemmer® PP-1000 (R²⁵=methyl, b=4-6, a=0), PP-500 (R²⁵=methyl, a=0, b=9), PP-800 (R²⁵=methyl, a=0, b=13); polyethyleneglycol polypropylene glycol methacrylate available under the product names Blemmer®50PEP-300 (R²⁵=methyl, a=3.5, b=2.5), 70PEP-350B (R²⁵=methyl, a=5, b=2); polyethyleneglycol acrylate available under the product names Blemmer® AE-90 (R²⁵=hydrogen, a=2, b=0), AE-200 (R²⁵=hydrogen, a=2, b=4.5), AE-400 (R²⁵=hydrogen, a=10, b=0); polypropyleneglycol acrylate available under the product names Blemmer® AP-150 (R²⁵=hydrogen, a=0, b=3), AP-400 (R²⁵=hydrogen, a=0, b=6), AP-550 (R²⁵=hydrogen, a=0, b=9). Blemmer® is a trademark of NOF Corporation, Tokyo, Japan.

Examples of alkoxylated semi-hydrophobic monomers under formula IXB include methoxypolyethyleneglycol methacrylate available under the product names Visiomer® MPEG 750 MA W (R²⁵=methyl, a=17, b=0), MPEG 1005 MA W (R²⁵=methyl, a=22, b=0), MPEG 2005 MA W (R²⁵=methyl, a=45, b=0), and MPEG 5005 MA W (R²⁵=methyl, a=113, b=0) from Evonik Röhm GmbH, Darmstadt, Germany); Bisomer MPEG 350 MA (R²⁵=methyl, a=8, b=0), and MPEG 550 MA (R²⁵=methyl, a=12, b=0) from GEO Specialty Chemicals, Ambler PA; Blemmer® PME-100 (R²⁵=methyl, a=2, b=0), PME-200 (R²⁵=methyl, a=4, b=0), PME-400 (R²⁵=methyl, a=9, b=0), PME-1000 (R²⁵=methyl, a=23, b=0), PME-4000 (R²⁵=methyl, a=90, b=0).

In one aspect, the alkoxylated semi-hydrophobic monomer set forth in formula X can be represented by the following formulas:

CH₂═CH—O—(CH₂)_{d}—O—(C₃H₆O)ₑ—(C₂H₄O)_{f}—H XA

CH₂═CH—CH₂—O—(C₃H₆O)_{g}—(C₂H₄O)ₕ—H XB

wherein d is an integer of 2, 3, or 4; e is an integer in the range of from about 1 to about 10 in one aspect, from about 2 to about 8 in another aspect, and from about 3 to about 7 in a further aspect; f is an integer in the range of from about 5 to about 50 in one aspect, from about 8 to about 40 in another aspect, and from about 10 to about 30 in a further aspect; g is an integer in the range of from 1 to about 10 in one aspect, from about 2 to about 8 in another aspect, and from about 3 to about 7 in a further aspect; and h is an integer in the range of from about 5 to about 50 in one aspect, and from about 8 to about 40 in another aspect; e, f, g, and h can be 0 subject to the proviso that e and f cannot be 0 at the same time, and g and h cannot be 0 at the same time.

Monomers under formulas XA and XB are commercially available under the trade names Emulsogen™ R109, R208, R307, RAL109, RAL208, and RAL307 sold by Clariant Corporation; BX-AA-E5P5 sold by Bimax, Inc.; and combinations thereof. EMULSOGEN R109 is a randomly ethoxylated/propoxylated 1,4-butanediol vinyl ether having the empirical formula CH₂═CH—O(CH₂)₄O(C₃H₆O)₄(C₂H₄O)₁₀H; Emulsogen R208 is a randomly ethoxylated/propoxylated 1,4-butanediol vinyl ether having the empirical formula CH₂═CH—O(CH₂)₄O(C₃H₆O)₄(C₂H₄O)₂₀H; Emulsogen R307 is a randomly ethoxylated/propoxylated 1,4-butanediol vinyl ether having the empirical formula CH₂═CH—O(CH₂)₄O(C₃H₆O)₄(C₂H₄O)₃₀H; Emulsogen RAL109 is a randomly ethoxylated/propoxylated allyl ether having the empirical formula CH₂═CHCH₂O (C₃H₆O)₄(C₂H₄O)₁₀H; Emulsogen RAL208 is a randomly ethoxylated/propoxylated allyl ether having the empirical formula CH₂═CHCH₂O (C₃H₆O)₄(C₂H₄O)₂₀H; Emulsogen RAL307 is a randomly ethoxylated/propoxylated allyl ether having the empirical formula CH₂═CHCH₂O (C₃H₆O)₄(C₂H₄O)₃₀H; and BX-AA-E5P5 is a randomly ethoxylated/propoxylated allyl ether having the empirical formula CH₂═CHCH₂O (C₃H₆O)₅(C₂H₄O)₅H.

The use of the alkoxylated semi-hydrophobic monomers is optional. These monomers can be utilized to tailor the hydrophilicity and/or hydrophobicity of the polymers in which they are included. For example, mid-section portions rich in ethylene oxide moieties are more hydrophilic while mid-section portions rich in propylene oxide moieties are more hydrophobic. By adjusting the relative amounts of ethylene oxide to propylene oxide moieties present in these monomers the hydrophilic and hydrophobic properties of the polymers in which these monomers are included can be tailored as desired.

In one embodiment, the amount of the alkoxylated semi-hydrophobic monomer utilized in the preparation of the polymers of the disclosed technology ranges from 0 to about 15 weight percent in one aspect, from about 0.5 to about 10 weight percent in another aspect, and from about 1, 2 or 3 to about 5 weight percent in a further aspect, based on the weight of the total monomers.

Crosslinking Monomer (F)

In one embodiment, the polymers useful in the practice of the disclosed technology are optionally prepared from a crosslinking monomer. The crosslinking monomer(s) is utilized to polymerize covalent crosslinks into the polymer backbone. The crosslinking agents are conventional crosslinking monomers containing at least two ethylenically polymerizable unsaturated moieties. These are relatively low molecular weight compounds (less than 300 Daltons). In one aspect, the crosslinking monomer is a polyunsaturated compound containing at least 2 unsaturated moieties. In another aspect, the crosslinking monomer contains at least 3 unsaturated moieties. Exemplary polyunsaturated compounds include di(meth)acrylate compounds such as ethylene glycol di(meth)acrylate, polyethylene glycol di(meth)acrylate, triethylene glycol di(meth)acrylate, 1,3-butylene glycol di(meth)acrylate, 1,6-butylene glycol di(meth)acrylate, 1,6-hexanediol di(meth)acrylate, neopentyl glycol di(meth)acrylate, 1,9-nonanediol di(meth)acrylate, 2,2′-bis(4-(acryloxy-propyloxyphenyl)propane, and 2,2′-bis(4-(acryloxydiethoxy-phenyl)propane; tri(meth)acrylate compounds such as, trimethylolpropane tri(meth)acrylate, trimethylolethane tri(meth)acrylate, and tetramethylolmethane tri(meth)acrylate; tetra(meth)acrylate compounds such as ditrimethylolpropane tetra(meth)acrylate, tetramethylolmethane tetra(meth)acrylate, and pentaerythritol tetra(meth)acrylate; hexa(meth)acrylate compounds such as dipentaerythritol hexa(meth)acrylate; allyl compounds such as allyl (meth)acrylate, diallylphthalate, diallyl itaconate, diallyl fumarate, and diallyl maleate; polyallyl ethers of sucrose having from 2 to 8 allyl groups per molecule, polyallyl ethers of pentaerythritol such as pentaerythritol diallyl ether, pentaerythritol triallyl ether, and pentaerythritol tetraallyl ether, and combinations thereof; polyallyl ethers of trimethylolpropane such as trimethylolpropane diallyl ether, trimethylolpropane triallyl ether, and combinations thereof. Other suitable polyunsaturated compounds include divinyl glycol, divinyl benzene, and methylenebisacrylamide.

In another aspect, suitable polyunsaturated monomers can be synthesized via an esterification reaction of a polyol made from ethylene oxide or propylene oxide or combinations thereof with unsaturated anhydride such as maleic anhydride, citraconic anhydride, itaconic anhydride, or an addition reaction with unsaturated isocyanate such as 3-isopropenyl-α-α-dimethylbenzene isocyanate.

Mixtures of two or more of the foregoing polyunsaturated compounds can also be utilized to crosslink the HASE polymers of the disclosed technology.

In one embodiment of the disclosed technology, the amount of the crosslinking monomer ranges from 0 to about 1 weight percent in one aspect, from about 0.01 to about 0.75 weight percent in another aspect, from about 0.1 to about 0.5 in still another aspect, and from about 0.15 to about 0.3 weight percent in a still further aspect, all weight percentages are based on the total weight of the monounsaturated monomers utilized to prepare the HASE polymers of the disclosed technology. Stated another way, the amount of conventional crosslinking monomer discussed below can be calculated on the basis of parts by wt. (100% active material) per 100 parts by wt. (100% active material) of total monounsaturated monomers utilized to prepare the polymer of the disclosed technology.

In one embodiment the HASE polymers of the present technology are prepared from a monomer mixture that is devoid of any polyunsaturated monomers (e.g., conventional crosslinkers) other than the amphiphilic macromonomers described herein

HASE Polymer Synthesis

The HASE polymers of the disclosed technology can be made using conventional free-radical emulsion polymerization techniques. The polymerization processes are carried out in the absence of oxygen under an inert atmosphere such as nitrogen. The polymerization can be carried out in a suitable solvent system such as water. Minor amounts of a hydrocarbon solvent, organic solvent, as well as mixtures thereof can be employed. The polymerization reactions are initiated by any means which results in the generation of a suitable free-radical. Thermally derived radicals, in which the radical species is generated from thermal, homolytic dissociation of peroxides, hydroperoxides, persulfates, percarbonates, peroxyesters, hydrogen peroxide and azo compounds can be utilized. The initiators can be water soluble or water insoluble depending on the solvent system employed for the polymerization reaction.

The initiator compounds can be utilized in an amount of up to 30 percent by weight in one aspect, 0.01 to 10 percent by weight in another aspect, and 0.2 to 3 percent by weight in a further aspect, based on the total weight of the dry polymer.

Exemplary free radical water soluble initiators include, but are not limited to, inorganic persulfate compounds, such as ammonium persulfate, potassium persulfate, and sodium persulfate; peroxides such as hydrogen peroxide, benzoyl peroxide, acetyl peroxide, and lauryl peroxide; organic hydroperoxides, such as cumene hydroperoxide and t-butyl hydroperoxide; organic peracids, such as peracetic acid, and water soluble azo compounds, such as 2,2′-azobis(tert-alkyl) compounds having a water solubilizing substituent on the alkyl group. Exemplary free radical oil soluble compounds include, but are not limited to 2,2′-azobisisobutyronitrile, and the like. The peroxides and peracids can optionally be activated with reducing agents, such as sodium bisulfite, sodium formaldehyde, or ascorbic acid, transition metals, hydrazine, and the like.

In one aspect, azo polymerization catalysts include the Vazo® free-radical polymerization initiators, available from DuPont, such as Vazo® 44 (2,2′-azobis(2-(4,5-dihydroim idazolyl)propane), Vazo® 56 (2,2′-azobis(2-methylpropionam idine) dihydrochloride), Vazo® 67 (2,2′-azobis(2-methylbutyronitrile)), and Vazo® 68 (4,4′-azobis(4-cyanovaleric acid)), and VA-086 (2,2′-Azobis[2-methyl-N-(2-hydroxyethyl)propionam ide]) from Wako Chemicals.

In emulsion polymerization processes, it can be advantageous to stabilize the monomer/polymer droplets or particles by means of surface active auxiliaries. Typically, these are emulsifiers or protective colloids. Emulsifiers used can be anionic, nonionic, cationic or amphoteric. Examples of anionic emulsifiers are alkylbenzenesulfonic acids, sulfonated fatty acids, sulfosuccinates, fatty alcohol sulfates, alkylphenol sulfates and fatty alcohol ether sulfates. Examples of usable nonionic emulsifiers are alkylphenol ethoxylates, primary alcohol ethoxylates, fatty acid ethoxylates, alkanolamide ethoxylates, fatty amine ethoxylates, EO/PO block copolymers and alkylpolyglucosides. Examples of cationic and amphoteric emulsifiers used are quaternized amine alkoxylates, alkylbetaines, alkylamidobetaines and sulfobetaines.

Examples of typical protective colloids are cellulose derivatives, polyethylene glycol, polypropylene glycol, copolymers of ethylene glycol and propylene glycol, polyvinyl acetate, poly(vinyl alcohol), partially hydrolyzed poly(vinyl alcohol), polyvinyl ether, starch and starch derivatives, dextran, polyvinylpyrrolidone, polyvinylpyridine, polyethyleneimine, polyvinylimidazole, polyvinylsuccinimide, polyvinyl-2-methylsuccinimide, polyvinyl-1,3-oxazolid-2-one, polyvinyl-2-methylimidazoline and maleic acid or anhydride copolymers. The emulsifiers or protective colloids are customarily used in concentrations from 0.05 to 20 weight percent, based on the weight of the total monomers.

Optionally, the use of known redox initiator systems as polymerization initiators can be employed. Such redox initiator systems include an oxidant (initiator) and a reductant. Suitable oxidants include, for example, hydrogen peroxide, sodium peroxide, potassium peroxide, t-butyl hydroperoxide, t-amyl hydroperoxide, cumene hydroperoxide, sodium perborate, perphosphoric acid and salts thereof, potassium permanganate, and ammonium or alkali metal salts of peroxydisulfuric acid, typically at a level of 0.01 to 3.0 percent by weight, based on dry polymer weight, are used. Suitable reductants include, for example, alkali metal and ammonium salts of sulfur-containing acids, such as sodium sulfite, bisulfite, thiosulfate, hydrosulfite, sulfide, hydrosulfide or dithionite, formadinesulfinic acid, hydroxymethanesulfonic acid, acetone bisulfite, amines such as ethanolamine, glycolic acid, glyoxylic acid hydrate, ascorbic acid, isoascorbic acid, lactic acid, glyceric acid, malic acid, 2-hydroxy-2-sulfinatoacetic acid, tartaric acid and salts of the preceding acids typically at a level of 0.01 to 3.0 percent by weight, based on dry polymer weight, is used. In one aspect, combinations of peroxodisulfates with alkali metal or ammonium bisulfites can be used, for example, ammonium peroxodisulfate and ammonium bisulfite. In another aspect, combinations of hydrogen peroxide containing compounds (t-butyl hydroperoxide) as the oxidant with ascorbic or erythorbic acid as the reductant can be utilized. The ratio of peroxide-containing compound to reductant is within the range from 30:1 to 0.05:1.

The polymerization reaction can be carried out at temperatures ranging from 20 to 200° C. in one aspect, from 50 to 150° C. in another aspect, and from 60 to 100° C. in a further aspect.

The polymerization can be carried out the presence of a chain transfer agent (G). Suitable chain transfer agents include, but are not limited to, thio- and disulfide containing compounds, such as C₁-C₁₈ alkyl mercaptans, such as tert-butyl mercaptan, n-octyl mercaptan, n-dodecyl mercaptan, tert-dodecyl mercaptan hexadecyl mercaptan, octadecyl mercaptan; mercaptoalcohols, such as 2-mercaptoethanol, 2-mercaptopropanol; mercaptocarboxylic acids, such as mercaptoacetic acid and 3-mercaptopropionic acid; mercaptocarboxylic acid esters, such as butyl thioglycolate, isooctyl thioglycolate, dodecyl thioglycolate, isooctyl 3-mercaptopropionate, and butyl 3-mercaptopropionate; thioesters; C₁-C₁₈ alkyl disulfides; aryldisulfides; polyfunctional thiols such as trimethylolpropane-tris-(3-mercaptopropionate), pentaerythritol-tetra-(3-mercaptopropionate), pentaerythritol-tetra-(thioglycolate), pentaerythritol-tetra-(thiolactate), dipentaerythritol-hexa-(thioglycolate), and the like; phosphites and hypophosphites; C₁-C₄ aldehydes, such as formaldehyde, acetaldehyde, propionaldehyde; haloalkyl compounds, such as carbon tetrachloride, bromotrichloromethane, and the like; hydroxylammonium salts such as hydroxylammonium sulfate; formic acid; sodium bisulfite; isopropanol; and catalytic chain transfer agents such as, for example, cobalt complexes (e.g., cobalt (II) chelates).

The chain transfer agents are generally used in amounts ranging from about 0.05 to about 10 percent by weight in one aspect, from about 0.1 to about 5 weight percent in another aspect, and from about 0.5 to about 1 weight percent in a further aspect, based on the total weight of the monomers present in the polymerization medium.

Emulsion Process

In one exemplary aspect of the disclosed technology, the HASE polymer of the present technology is polymerized via an emulsion process. The emulsion process can be conducted in in a single reactor or in multiple reactors as is well-known in the art. The monomers can be added as a batch mixture or each monomer can be metered into the reactor in a staged process. A typical mixture in emulsion polymerization comprises water, monomer(s), an initiator (usually water-soluble) and an emulsifier. The monomers may be emulsion polymerized in a single-stage, two-stage or multi-stage polymerization process according to well-known methods in the emulsion polymerization art. In a two-stage polymerization process, the first stage monomers are added and polymerized first in the aqueous medium, followed by addition and polymerization of the second stage monomers. The aqueous medium optionally can contain an organic solvent. If utilized the organic solvent is less than about 5 weight percent of the aqueous medium. Suitable examples of water-miscible organic solvents include, without limitation, esters, alkylene glycol ethers, alkylene glycol ether esters, lower molecular weight aliphatic alcohols, and the like.

To facilitate emulsification of the monomer mixture, the emulsion polymerization is carried out in the presence of at least one surfactant. In one embodiment, the emulsion polymerization is carried out in the presence of surfactant (active weight basis) ranging in the amount of about 0.2 to about 5 percent by weight in one aspect, from about 0.5 to about 3 percent by weight in another aspect, and from about 1 to about 2 percent by weight in a further aspect, based on a total monomer weight basis. The emulsion polymerization reaction mixture also includes one or more free radical initiators which are present in an amount ranging from about 0.01 to about 3 percent by weight, based on total monomer weight. The polymerization can be performed in an aqueous or aqueous alcohol medium. Surfactants for facilitating the emulsion polymerization include anionic, nonionic, amphoteric, and cationic surfactants, as well as mixtures thereof. Most commonly, anionic and nonionic surfactants can be utilized as well as mixtures thereof.

Suitable anionic surfactants for facilitating emulsion polymerizations are well known in the art and include, but are not limited to (C₆-C₁₈) alkyl sulfates, (C₆-C₁₈) alkyl ether sulfates (e.g., sodium lauryl sulfate and sodium laureth sulfate), amino and alkali metal salts of dodecylbenzenesulfonic acid, such as sodium dodecyl benzene sulfonate and dimethylethanolamine dodecylbenzenesulfonate, sodium (C₆-C₁₆) alkyl phenoxy benzene sulfonate, disodium (C₆-C₁₆) alkyl phenoxy benzene sulfonate, disodium (C₆-C₁₆) di-alkyl phenoxy benzene sulfonate, disodium laureth-3 sulfosuccinate, sodium dioctyl sulfosuccinate, sodium di-sec-butyl naphthalene sulfonate, disodium dodecyl diphenyl ether sulfonate, disodium n-octadecyl sulfosuccinate, phosphate esters of branched alcohol ethoxylates, and the like.

Nonionic surfactants suitable for facilitating emulsion polymerizations are well known in the polymer art, and include, without limitation, linear or branched C₈-C₃₀ fatty alcohol ethoxylates, such as capryl alcohol ethoxylate, lauryl alcohol ethoxylate, myristyl alcohol ethoxylate, cetyl alcohol ethoxylate, stearyl alcohol ethoxylate, cetearyl alcohol ethoxylate, sterol ethoxylate, oleyl alcohol ethoxylate, and, behenyl alcohol ethoxylate; alkylphenol alkoxylates, such as octylphenol ethoxylates; and polyoxyethylene polyoxypropylene block copolymers, and the like. Additional fatty alcohol ethoxylates suitable as non-ionic surfactants are described below. Other useful nonionic surfactants include C₈-C₂₂ fatty acid esters of polyoxyethylene glycol, ethoxylated mono- and diglycerides, sorbitan esters and ethoxylated sorbitan esters, C₈-C₂₂ fatty acid glycol esters, block copolymers of ethylene oxide and propylene oxide, and combinations thereof. The number of ethylene oxide units in each of the foregoing ethoxylates can range from 2 and above in one aspect, and from 2 to about 150 in another aspect.

Optionally, other emulsion polymerization additives and processing aids which are well known in the emulsion polymerization art, such as auxiliary emulsifiers, protective colloids, solvents, buffering agents, chelating agents, inorganic electrolytes, polymeric stabilizers, biocides, and pH adjusting agents can be included in the polymerization system.

In one embodiment of the disclosed technology, the protective colloid or auxiliary emulsifier is selected from poly(vinyl alcohol) (PVA) that has a degree of hydrolysis ranging from about 80 to 95 percent in one aspect, and from about 85 to 90 percent in another aspect. A commercially available PVA is Selvol™ 502 and 203 marketed by Sekisui Specialty Chemicals.

In a typical two stage emulsion polymerization, a mixture of the monomers is added to a first reactor under inert atmosphere to a solution of emulsifying surfactant (e.g., anionic surfactant) in water. Optional processing aids can be added as desired (e.g., protective colloids, auxiliary emulsifier(s)). The contents of the reactor are agitated to prepare a monomer emulsion. To a second reactor equipped with an agitator, an inert gas inlet, and feed pumps are added under inert atmosphere a desired amount of water and additional anionic surfactant and optional processing aids. The contents of the second reactor are heated with mixing agitation. After the contents of the second reactor reaches a temperature in the range of about 55 to 98° C., a free radical initiator is injected into the so formed aqueous surfactant solution in the second reactor, and the monomer emulsion from the first reactor is gradually metered into the second reactor over a period typically ranging from about one half to about four hours. The reaction temperature is controlled in the range of about 45 to about 95° C. After completion of the monomer addition, an additional quantity of free radical initiator can optionally be added to the second reactor, and the resulting reaction mixture is typically held at a temperature of about 45 to 95° C. for a time period sufficient to complete the polymerization reaction to obtain the polymer emulsion.

The polymer emulsion product can be prepared to contain about 1 percent to about 60 percent total active polymer solids (TS) in one aspect, from about 10 percent to about 50 percent total polymer solids in another aspect, from about 15 percent to about 45 percent total polymer solids in still another aspect, from about 25 to about 35 percent in a further aspect, and from about 30 to 32 percent, based on the weight of the emulsion.

In one aspect, the polymer product is a random copolymer and has number average molecular weight as measured by gel permeation chromatography (GPC) calibrated with a poly(methyl methacrylate) (PMMA) ranging from above about 500,000 to at least about a billion daltons or more in one aspect, from about 600,000 to about 4.5 billion daltons in another aspect, and from about 1,000,000 to about 3,000,000 daltons in a further aspect, and from about 1,500,000 to about 2,000,000 daltons in a still further aspect (see TDS-222, Oct. 15, 2007, Lubrizol Advanced Materials, Inc., which is herein incorporated by reference).

Prior to any neutralization, the polymer emulsions, as produced, typically have a pH in the range of about 2 to not more than about 5.5, a Brookfield viscosity of not more than about 100 milli-Pascal seconds (mPa·s) at ambient room temperature (spindle No. 2, 20 rpm).

Optionally, the produced polymer emulsions can be further processed by adjusting the pH to a value in the range of about 3 to about 7.5 or greater, if an alkaline pH is desired, with alkaline materials, preferably alkali metal hydroxides, organic bases, and the like. The polymer emulsions typically swell to a viscosity greater than about 100 mPa·s and form viscous solutions or gels at neutral to alkaline pH, and the polymers are generally substantially stable at such pH values, even at pH values greater than about 12. The polymer emulsions can be diluted with water or solvent, or concentrated by evaporation of a portion of the water. Alternatively, the obtained polymer emulsion may be substantially dried to a powder or crystalline form by utilizing equipment well known in the art, such as, for example, a spray drier, a drum drier, or a freeze drier.

The HASE polymers can be utilized by incorporating various known additives and conventional adjuvants and solvents into the emulsion product as needed, to achieve the intended form for use of the final composition without altering or adversely affecting the performance or properties of the polymer. Alternatively, the polymer can be incorporated as an ingredient into a formulation, preferably in a liquid form, employing conventional mixing equipment.

The HASE polymer of the disclosed technology can be employed as a film former. When the glass transition temperature (Tg) of a selected HASE polymer film former is substantially above ambient room temperature, the Tg of the HASE film former can be adjusted to achieve a desired Tg by including additives in the formulation, such as coalescing agents, plasticizers and mixtures thereof. Such additives can assist in film formation by lowering the Tg of the HASE polymer to the ambient room temperature or desired temperature.

The HASE polymer of the disclosed technology can be utilized, for example, without being limited thereto, as a rheology modifier, suspending agent, film former, thickener, stabilizer, emulsifier, solubilizer, and the like, in formulated compositions for personal care products, topical health care products, household care products, institutional and industrial (I&I) products and industrial processes. The foregoing products can typically contain various additives and conventional adjuvants as are well known in the art, including, without being limited thereto, acidifying or alkalizing pH adjusting agents and buffering agents; fixatives and auxiliary film formers, such as gums, resins, polymers of synthetic or natural origin, and the like; auxiliary rheology modifiers, such as viscosity increasing polymeric thickeners or gellants, additives, such as emulsifiers, emulsion stabilizers, waxes, dispersants, and the like, and viscosity control agents, such as solvents, electrolytes, and the like; hair and skin conditioning agents, such as antistatic agents, synthetic oils, vegetable or animal oils, silicone oils, monomeric or polymeric quaternized ammonium salts, emollients, humectants, lubricants, sunscreen agents, and the like; chemical hair waving or straightening agents; hair colorants, such as pigments and dyes for temporary, semipermanent, or permanent hair dyeing; surfactants, such as anionic, cationic, nonionic, amphoteric and zwitterionic surfactants; polymer film modifying agents, such as plasticizers, humectants, tackifiers, detackifiers, wetting agents and the like, product finishing agents, such as chelating agents, opacifiers, pearlescing agents, preservatives, fragrances, solubilizers, colorants, such as pigments and dyes, UV absorbers, and the like; propellants (water-miscible or water-immiscible), such as fluorinated hydrocarbons, liquid volatile hydrocarbons, compressed gases, and the like; and mixtures thereof.

In one embodiment, the HASE polymers of the disclosed technology can be utilized to suspend particulate materials and insoluble droplets within an aqueous composition. Such fluids are useful in the oil and gas, personal care, homecare, coatings and inks and adhesive/binder industries.

The stable compositions maintain a smooth, acceptable rheology with good shear thinning properties without significant increases or decreases in viscosity, with no phase separation, e.g., settling or creaming out (rising to the surface), or loss of clarity over extended periods of time, such as for at least one month at 45° C.

In the coatings, inks, and adhesive/binder industries the HASE polymers of the disclosed technology are useful to adjust the viscosity of a liquid composition to: a) control or minimize settling or creaming of solid particles, dispersed liquids, trapped gases, and particulates (aid in suspension) that are more dense or less dense than the continuous media (often water based); b) to control application viscosity of continuous or discontinuous layers of a coating, ink, or adhesive to a substrate; c) to minimize movement or flow of coatings, inks, or adhesives immediately prior to application or in the time after application until the coating, ink, or adhesive forms a continuous gelled polymer; e) reduce splatter and misting in some application processes; f) etc., to facilitate optimal storage, application ease, and final surface finish in those applications. The coatings, inks and adhesives may comprise particulate or fibrous fillers, pigments, dyes, other polymers, surfactants and/or dispersants, coalescing aids, plasticizers, biocides and other conventional additives employed in coatings, inks, and adhesives. The coatings can be used on metals, plastics, wood, masonry, textiles, papers, etc. The inks can be used on any ink substrates such as paper, polymers, wovens, nonwovens, films, etc. The HASE polymer can contribute to both viscosity control and optical clarity (helping color intensity of pigmented compositions) of the coating, ink, or adhesive.

In the personal care, topical health care, and homecare industries the HASE polymers of the disclosed technology can be utilized as rheology modifiers to thicken aqueous and surfactant containing compositions as well as to improve the yield stress (stable suspension of insoluble and particulate materials) properties of surfactant containing compositions, hair and skin care compositions, and cosmetics. The HASE polymers can be utilized to suspend insoluble silicones, opacifiers and pearlescent agents (e.g., mica, coated mica), pigments, exfoliants, anti-dandruff agents, clay, swellable clay, laponite, gas bubbles (aesthetic air bubbles), liposomes, microsponges, cosmetic beads, fragrance microcapsules, fragrance particles, benefit agent containing microcapsules and particles, cosmetic microcapsules, and flakes. The HASE polymers of the disclosed technology can stabilize these materials in suspension for at least one month at 23° C. in one aspect, at least 6 months in another aspect, and at least one year in a further aspect.

Compositions for personal care and topical health care can comprise any cosmetic, toiletry, and topical pharmaceutical formulation that requires rheology modification or thickening known from the cosmetic and pharmaceutical literature. Typical personal care formulations that can include the HASE polymers as a rheology modifier include, without being limited thereto, shampoos, chemical and non-chemical hair curling and hair straightening products, hair style maintenance products, emulsion lotions and creams for the nails, hands, feet, face, scalp, and body, hair dyes, face and body makeup, nail care products, astringents, deodorants, antiperspirants, depilatories, skin-protective creams and lotions, such as sunscreens, skin and body cleansers, skin conditioners, skin toners, skin firming compositions, liquid soaps, soap bars, bath products, shaving products, and the like. Formulated compositions for topical health care that are applied to the skin and mucous membranes for cleansing or soothing are compounded with many of the same physiologically tolerable cosmetic ingredients and chemically inert ingredients employed for personal care products in the same product forms, differing primarily in the purity grade of ingredients and by the presence of topically active medicaments. For example, topical health care products include oral hygiene products, such as toothpastes, oral suspensions, and mouth care products, which can be classified as pharmaceuticals or over-the-counter products, and include pharmacosmetics, which contain phytopharmaceutic or nutraceutical ingredients.

Compositions for personal care and topical health care can be in the form of, without being limited thereto, liquids, such as rinses, gels, sprays, emulsions, such as lotions and creams, shampoos, pomades, foams, ointments, tablets, sticks, such as lip care products, makeup, and suppositories, and like products, which are applied to skin and hair and remain in contact therewith until removed as by rinsing with water or washing with shampoo or soap. Gels can be soft, stiff, or squeezable. Emulsions can be oil-in-water, water-in-oil, or multiphase. Sprays can be non-pressurized aerosols delivered from manually pumped finger-actuated sprayers or can be pressurized aerosols. The HASE polymer can be formulated in an aerosol composition, such as in a spray, mousse, or foam forming formulation, where a chemical or gaseous propellant is required. Physiologically and environmentally tolerable propellants, such as compressed gases, fluorinated hydrocarbons and liquid volatile hydrocarbons, and the amounts and suitable combinations to be used, are well known in the cosmetic and pharmaceutical art and literature.

An extensive listing of personal care and cosmetic ingredients and their functions, for example, appears in the INCI Dictionary, generally, and in Vol. 2, Section 4 of the Seventh Edition, in particular, incorporated herein by reference. Those skilled in the art of formulating personal care and health care products recognize that some ingredients are multifunctional and, hence, can serve more than one purpose in the formulation. Thus, the amount of HASE polymer employed as a personal care or health care product component is not limited, as long as the purpose and properties of the formulated composition performs its intended function.

Typical household care, and I&I care products that can contain the HASE polymers of the disclosed technology as a rheology modifier include, without being limited thereto, surface cleansers for kitchen and bathroom counter tops, tiled surfaces, and utilities, including appliances employed or located therein, toilet cleaners, including toilet bowl rim gels, floor cleansers, wall cleansers, polishes, air freshener gels, detergents, treatments and cleansers for dishes and laundry, such as fabric softener, spot reducer, fabric treatments, and the like.

The HASE polymers and polymer compositions according to the present technology are pH-responsive. At the lower pH levels at which the emulsion polymerization takes place, i.e., pH levels of 5 or less, the composition is relatively thin or non-viscous. When the pH of the polymer dispersion is neutralized or adjusted by addition of an alkaline material (base) to a pH of about 5.5 or more in one aspect, and from about 6.5 to about 11 in another aspect, the composition thickens substantially. Viscosity increases as the polymer dissolves partially or completely in the aqueous phase of the composition. Neutralization can occur in situ when the emulsion polymer is blended with the base and added to the aqueous phase. Or, if desired for a given application, neutralization can be carried out when blending with an aqueous product.

Many types of alkaline neutralizing agents can be used to neutralize the polymer, including inorganic and organic bases, and combinations thereof. Examples of inorganic bases include but are not limited to the alkali metal hydroxides (especially sodium, potassium, and ammonium), and alkali metal salts of inorganic acids, such as sodium borate (borax), sodium phosphate, sodium pyrophosphate, and the like; and mixtures thereof. Examples of organic bases include, but are not limited to, triethanolamine (TEA), diisopropanolamine, triisopropanolamine, aminomethyl propanol, dodecylamine, cocamine, oleamine, morpholine, triamylamine, triethylamine, tetrakis(hydroxypropyl)ethylenediamine, L-arginine, aminomethyl propanol, tromethamine (2-amino 2-hydroxymethyl-1,3-propanediol), and PEG-15 cocamine. Alternatively, other alkaline materials can be used alone or in combination with the above-mentioned inorganic and organic bases. Such materials include surfactants, surfactant mixtures, pre-neutralized surfactants or materials that when combined in a composition containing a polymer of the technology is capable of neutralizing or partially neutralizing the carboxyl groups on the polymer backbone. Any material capable of increasing the pH of the composition is suitable.

Compositions comprising the HASE polymer of the disclosed technology have a desired pH range of about 4 to about 12 in one aspect, from about 6 to about 7.5 in another aspect, and from about 6.5 to about 7 in a further aspect.

The amount of the HASE polymer that can be employed in the foregoing compositions can be determined by person skilled in the formulation art. Thus, as long as the physicochemical and functional properties of a desired product are achieved, a useful amount of polymer on a total composition weight basis, typically can vary in the range of from about 0.01 to about 25 weight percent in one aspect, from about 0.1 to about 15 weight percent in another aspect, from about 0.5 to about 10 weight percent in a further aspect, and from about 1 to about 5 weight percent in a still further aspect based on the weight of the total composition (all polymer weights based on 100 percent active polymer solids).

In one embodiment, the disclosed technology concerns a personal care composition comprising water, one or more surfactants and a HASE polymer according to the disclosed technology. In one aspect of the present technology, the disclosed HASE polymers can be formulated with surfactants to provide thickened surfactant containing compositions. The surfactant can be selected from at least one anionic surfactant, at least one cationic surfactant, at least one amphoteric or zwitterionic surfactant, at least one nonionic surfactant, and mixtures thereof.

In one embodiment, the disclosed technology concerns a personal care composition comprising water, one or more surfactants and at least one HASE polymer according to the disclosed technology, wherein the one or more surfactants are present over a wide concentration ranging from about 3 to about 25 wt. % in one aspect, from about 5 to about 20 wt. % in another aspect, and from about 8 to about 16 wt. % in a further aspect (100% active material based on the weight of the total composition), and wherein the at least one HASE polymer is present from about 1 to about 5 wt. % in one aspect, from about 1.5 to about 4 wt. % in another aspect, and from about 1.75 to about 3 wt. % in a further aspect (100% active material based on the total weight of the composition), and wherein such compositions have an ideal viscosity ranging from about 1,000 to about 35,000 mPa-s in one aspect, from about 3,000 to about 25,000 mPa-s in another aspect, from about 5,000 to about 20,000 mPa-s in still another aspect, and from about 8,000 to about 15,000 mPa-s in a further aspect (as measured on a Brookfield rotating spindle viscometer, Model RVT, at about 20 rpm, at ambient room temperature ranging between 20 to 25° C.), and wherein such compositions have a yield stress greater than 0 Pa in one aspect, ranging from about 1 to about 9 Pa in another aspect, from about 10 to about 20 Pa, in still another aspect, from about 21 to about 30 Pa in a further aspect, and greater than about 30 Pa in a still further aspect, and wherein such compositions are capable of suspending insoluble and/or particulate materials for prolonged periods of time at an elevated temperature of 45° C. or greater, for at least about 1 week in one aspect, at least about 1 month in another aspect, at least about 3 months in a further aspect.

In one embodiment, the disclosed technology concerns a personal care composition comprising water, one or more sulfate-free surfactants and a HASE polymer according to the disclosed technology. Generally speaking, it is difficult to obtain high clarity and good bead suspension (yield stress) when thickening compositions containing sulfate-free surfactant systems. The polymers of the disclosed technology are able to thicken formulations containing sulfate-free surfactants while providing good clarity and the stable suspension of insoluble and particulate materials within such formulations.

Non-limiting examples of anionic surfactants are disclosed in McCutcheon's Detergents and Emulsifiers, North American Edition, 1998, published by Allured Publishing Corporation; and McCutcheon's, Functional Materials, North American Edition (1992); both of which are incorporated by reference herein in their entirety. The anionic surfactant can be any of the anionic surfactants known or previously used in the art of aqueous surfactant compositions, including synthetic surfactants (syndets) and fatty acid soaps.

Suitable anionic syndet surfactants include but are not limited to alkyl sulfates, alkyl ether sulfates, alkyl sulfonates, alkylaryl sulfonates, alkenyl and hydroxyalkyl alpha-olefin sulfonates, and mixtures thereof, alkylamide sulfonates, alkarylpolyether sulphates, alkylamidoether sulphates, alkyl and alkenyl monoglyceryl ether sulfates, alkyl and alkenyl monoglyceride sulfates, alkyl and alkenyl monoglyceride sulfonates, alkyl sulfoacetates, alkyl and alkenyl succinates, alkyl and alkenyl sulfosuccinates, alkyl and alkenyl sulfosuccinamates, alkyl and alkenyl ether sulfosuccinates, alkyl and alkenyl amidosulfosuccinates; alkyl and alkenyl sulphoacetates, alkyl and alkenyl phosphates, alkyl and alkenyl ether phosphates, alkyl and alkenyl carboxylates, alkyl and alkenyl ether carboxylates, alkyl and alkenyl amidoethercarboxylates, N-alkylamino acids, N-acyl amino acids, alkyl peptides, N-acyl taurates, acyl isethionates, carboxylate salts wherein the acyl group is derived from fatty acids; and the alkali metal, alkaline earth metal, ammonium, amine, and triethanolamine salts thereof.

In one aspect, the cation moiety of the foregoing salts is selected from sodium, potassium, magnesium, ammonium, mono-, di- and triethanolamine salts, and mono-, di-, and tri-isopropylamine salts. The alkyl and acyl groups of the foregoing surfactants contain from about 6 to about 24 carbon atoms in one aspect, from 8 to 22 carbon atoms in another aspect, and from about 12 to 18 carbon atoms in a further aspect, and can be saturated or unsaturated. The aryl groups in the surfactants are selected from phenyl or benzyl. The ether containing surfactants set forth above can contain from 1 to 10 ethylene oxide and/or propylene oxide units per surfactant molecule in one aspect, and from 1 to 3 ethylene oxide units per surfactant molecule in another aspect.

Examples of suitable anionic surfactants include but are not limited to the sodium, potassium, lithium, magnesium, ammonium, and triethanolamine lauryl sulfate, coco sulfate, tridecyl sulfate, myrstyl sulfate, cetyl sulfate, cetearyl sulfate, stearyl sulfate, oleyl sulfate, and tallow sulfate; the sodium, potassium, lithium, magnesium, and ammonium salts of laureth sulfate, trideceth sulfate, myreth sulfate, C₁₂-C₁₃ pareth sulfate, C₁₂-C₁₄ pareth sulfate, and C₁₂-C₁₅ pareth sulfate, ethoxylated with 1, 2, 3, 4 or 5 moles of ethylene oxide; disodium lauryl sulfosuccinate, disodium laureth sulfosuccinate, sodium cocoyl isethionate, sodium C₁₂-C₁₆ olefin sulfonate, sodium laureth-6 carboxylate, sodium methyl cocoyl taurate, sodium dodecylbenzene sulfonate, sodium cocoyl sarcosinate, triethanolamine monolauryl phosphate, sodium lauryl sulfoacetate, and fatty acid soaps, including the sodium, potassium, ammonium, and triethanolamine salts of a saturated and unsaturated fatty acids containing from about 8 to about 22 carbon atoms.

The anionic fatty acid soaps are salts of fatty acids containing from about 8 to about 22 carbon atoms, and mixtures thereof. In another aspect, the fatty acid soap contains from about 10 to about 18 carbon atoms, and mixtures thereof. In a further aspect, the fatty acid soap contains from about 12 to about 16 carbon atoms, and mixtures thereof. The fatty acids utilized in the soaps can be saturated and unsaturated and can be derived from synthetic sources, as well as from the hydrolysis of fats and natural oils.

Exemplary saturated fatty acids include but are not limited to octanoic, decanoic, lauric, myristic, pentadecanoic, palmitic, margaric, steric, isostearic, nonadecanoic, arachidic, behenic, and the like, and mixtures thereof. Exemplary unsaturated fatty acids include but are not limited to myristoleic, palmitoleic, oleic, linoleic, linolenic, and the like, and mixtures thereof. The fatty acids can be derived from animal fat such as tallow, lard, poultry fat or from vegetable sources such as coconut oil, red oil, palm kernel oil, palm oil, cottonseed oil, linseed oil, sunflower seed oil, olive oil, soybean oil, peanut oil, corn oil, safflower oil, sesame oil, rapeseed oil, canola oil, and mixtures thereof.

The soap can be prepared by a variety of well-known means such as by the direct base neutralization of a fatty acid or mixtures thereof or by the saponification of suitable fats and vegetable oils or mixtures thereof with a suitable base. Exemplary bases include potassium hydroxide, potassium carbonate, sodium hydroxide and alkanol amines such as triethanolamine. Generally, the fat or oil is heated until liquefied and a solution of the desired base is added thereto. Soaps included in a composition utilized in the method of the disclosed technology can be made, for example, by a classic kettle process or modern continuous manufacturing process wherein natural fats and oils such as tallow or coconut oil or their equivalents are saponified with an alkali metal hydroxide using procedures well known to those skilled in the art. Alternatively, soaps can be made by the direct neutralization of free fatty acids such as lauric acid (C₁₂), myristic acid (C₁₄), palmitic acid (C₁₆), steric acid (C₁₈), isostearic (C₁₈), and mixtures thereof, with an alkali metal hydroxide or carbonate.

Amino acid based surfactants suitable in the practice of the present technology include surfactants represented by the formula:

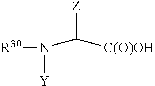

wherein R³⁰ represents a saturated or unsaturated hydrocarbon group having 10 to 22 carbon atoms or an acyl group containing a saturated or unsaturated hydrocarbon group having 9 to 22 carbon atoms, Y is hydrogen or methyl, Z is selected from hydrogen, —CH₃, —CH(CH₃)₂, —CH₂CH(CH₃)₂, —CH(CH₃)CH₂CH₃, —CH₂C₆H₅, —CH₂C₆H₄OH, —CH₂OH, —CH(OH)CH₃, —(CH₂)₄NH₂, —(CH₂)₃NHC(NH)NH₂, —CH₂C(O)O⁻M⁺, —(CH₂)₂C(O)O⁻M⁺. M is a salt forming cation. In one aspect, R³⁰ represents a radical selected from a linear or branched C₁₀ to C₂₂ alkyl group, a linear or branched C₁₀ to C₂₂ alkenyl group, an acyl group represented by R³¹C(O)—, wherein R³¹ is selected from a linear or branched C₉ to C₂₂ alkyl group, a linear or branched C₉ to C₂₂ alkenyl group. In one aspect, M⁺ is a cation selected from sodium, potassium, ammonium, and the ammonium salt of mono-, di, and triethanolamine (TEA).

The amino acid surfactants can be derived from the alkylation and acylation of α-amino acids such as, for example, alanine, arginine, aspartic acid, glutamic acid, glycine, isoleucine, leucine, lysine, phenylalanine, serine, tyrosine, and valine. Representative N-acyl amino acid surfactants are, but not limited to the mono- and di-carboxylate salts (e.g., sodium, potassium, ammonium and TEA) of N-acylated glutamic acid, for example, sodium cocoyl glutamate, sodium lauroyl glutamate, sodium myristoyl glutamate, sodium palmitoyl glutamate, sodium stearoyl glutamate, disodium cocoyl glutamate, disodium stearoyl glutamate, potassium cocoyl glutamate, potassium lauroyl glutamate, and potassium myristoyl glutamate; the carboxylate salts (e.g., sodium, potassium, ammonium and TEA) of N-acylated alanine, for example, sodium cocoyl alaninate, and TEA lauroyl alaninate; the carboxylate salts (e.g., sodium, potassium, ammonium and TEA) of N-acylated glycine, for example, sodium cocoyl glycinate, and potassium cocoyl glycinate; the carboxylate salts (e.g., sodium, potassium, ammonium and TEA) of N-acylated sarcosine, for example, sodium lauroyl sarcosinate, sodium cocoyl sarcosinate, sodium myristoyl sarcosinate, sodium oleoyl sarcosinate, and ammonium lauroyl sarcosinate; and mixtures of the foregoing surfactants.

The anionic surfactant component in the composition should be sufficient to provide the desired cleansing and lather performance, and generally ranges from about 2 to about 50 weight percent active material in one aspect, from about 8 to about 30 weight percent in another aspect, from about 10 to about 25 weight percent in still another aspect, and from about 12 to about 22 weight percent in a further aspect, all weight percentages are based on the weight of the total composition.

The cationic surfactants can be any of the cationic surfactants known or previously used in the art of aqueous surfactant compositions. Useful cationic surfactants can be one or more of those described, for example, in McCutcheon's Detergents and Emulsifiers, North American Edition, 1998, supra, and Kirk-Othmer, Encyclopedia of Chemical Technology, 4th Ed., Vol. 23, pp. 478-541, the contents of which are herein incorporated by reference. Suitable classes of cationic surfactants include but are not limited to alkyl amines, alkyl imidazolines, ethoxylated amines, quaternary compounds, and quaternized esters. In addition, alkyl amine oxides can function as a cationic surfactant at a low pH.

Alkylamine surfactants can be salts of primary, secondary and tertiary fatty C₁₂-C₂₂ alkylamines, substituted or unsubstituted, and substances sometimes referred to as “amidoamines”. Non-limiting examples of alkylamines and salts thereof include dimethyl cocamine, dimethyl palmitamine, dioctylamine, dimethyl stearamine, dimethyl soyamine, soyamine, myristyl amine, tridecyl amine, ethyl stearylamine, N-tallowpropane diamine, ethoxylated stearylamine, dihydroxy ethyl stearylamine, arachidylbehenylamine, dimethyl lauramine, stearylamine hydrochloride, soyamine chloride, stearylamine formate, N-tallowpropane diamine dichloride, and amodimethicone.

Non-limiting examples of amidoamines and salts thereof include stearamido propyl dimethyl amine, stearamidopropyl dimethylamine citrate, palmitamidopropyl diethylamine, and cocamidopropyl dimethylamine lactate.

Non-limiting examples of alkyl imidazoline surfactants include alkyl hydroxyethyl imidazoline, such as stearyl hydroxyethyl imidazoline, coco hydroxyethyl imidazoline, ethyl hydroxymethyl oleyl oxazoline, and the like.

Non-limiting examples of ethyoxylated amines include PEG-cocopolyamine, PEG-15 tallow amine, quaternium-52, and the like.

Among the quaternary ammonium compounds useful as cationic surfactants, some correspond to the general formula: (R³³R³⁴R³⁵R³⁶N+) E⁻, wherein R³³, R³⁴, R³⁵, and R³⁶ are independently selected from an aliphatic group having from 1 to about 22 carbon atoms, or an aromatic, alkoxy, polyoxyalkylene, alkylamido, hydroxyalkyl, aryl or alkylaryl group having 1 to about 22 carbon atoms in the alkyl chain; and E⁻ is a salt-forming anion such as those selected from halogen, (e.g., chloride, bromide), acetate, citrate, lactate, glycolate, phosphate, nitrate, sulfate, and alkylsulfate. The aliphatic groups can contain, in addition to carbon and hydrogen atoms, ether linkages, ester linkages, and other groups such as amino groups. The longer chain aliphatic groups, e.g., those of about 12 carbons, or higher, can be saturated or unsaturated. In one aspect, the aryl groups are selected from phenyl and benzyl.

Exemplary quaternary ammonium surfactants include, but are not limited to cetyl trimethylammonium chloride, cetylpyridinium chloride, dicetyl dimethyl ammonium chloride, dihexadecyl dimethyl ammonium chloride, stearyl dimethyl benzyl ammonium chloride, dioctadecyl dimethyl ammonium chloride, dieicosyl dimethyl ammonium chloride, didocosyl dimethyl ammonium chloride, dihexadecyl dimethyl ammonium chloride, dihexadecyl dimethyl ammonium acetate, behenyl trimethyl ammonium chloride, benzalkonium chloride, benzethonium chloride, and di(coconutalkyl) dimethyl ammonium chloride, ditallowdimethyl ammonium chloride, di(hydrogenated tallow) dimethyl ammonium chloride, di(hydrogenated tallow) dimethyl ammonium acetate, ditallowdimethyl ammonium methyl sulfate, ditallow dipropyl ammonium phosphate, and ditallow dimethyl ammonium nitrate.

At low pH, amine oxides can protonate and behave similarly to N-alkyl amines. Examples include, but are not limited to, dimethyl-dodecylamine oxide, oleyldi(2-hydroxyethyl) amine oxide, dimethyltetradecylamine oxide, di(2-hydroxyethyl)-tetradecylam ine oxide, dimethylhexadecylamine oxide, behenamine oxide, cocamine oxide, decyltetradecylamine oxide, dihydroxyethyl C₁₂-C₁₅ alkoxypropylamine oxide, dihydroxyethyl cocamine oxide, dihydroxyethyl lauramine oxide, dihydroxyethyl stearamine oxide, dihydroxyethyl tallowamine oxide, hydrogenated palm kernel amine oxide, hydrogenated tallowamine oxide, hydroxyethyl hydroxypropyl C₁₂-C₁₅ alkoxypropylamine oxide, lauramine oxide, myristamine oxide, cetylamine oxide, oleamidopropylamine oxide, oleamine oxide, palmitamine oxide, PEG-3 lauramine oxide, dimethyl lauramine oxide, potassium trisphosphonomethylamine oxide, soyamidopropylamine oxide, cocamidopropylamine oxide, stearamine oxide, tallowamine oxide, and mixtures thereof.

The amount of cationic surfactant active material will generally range from about 0.01 to about 10 weight percent in one aspect, from about 0.05 to about 7.5 weight percent in another aspect, and from about 0.1 to about 5 weight percent in a further aspect, based on the total weight of the composition.

The term “amphoteric surfactant” as used herein, is also intended to encompass zwitterionic surfactants, which are well known to formulators skilled in the art as a subset of amphoteric surfactants. Nonlimiting examples of amphoteric surfactants are disclosed McCutcheon's Detergents and Emulsifiers, North American Edition, supra, and McCutcheon's, Functional Materials, North American Edition, supra; both of which are incorporated by reference herein in their entirety. Suitable examples include but are not limited to betaines, sultaines, and alkyl amphocarboxylates. Other non-limiting examples of suitable zwitterionic or amphoteric surfactants are described in U.S. Pat. Nos. 5,104,646, and 5,106,609.

The betaines and sultaines useful in the present technology are selected from alkyl betaines, alkylamino betaines, and alkylamido betaines, as well as the corresponding sulfobetaines (sultaines) represented by the formulas:

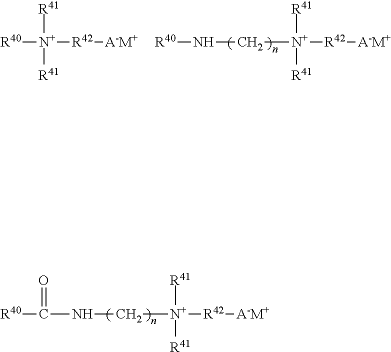

wherein R⁴⁰ is a C₇-C₂₂ alkyl or alkenyl group, each R⁴¹ independently is a C₁-C₄ alkyl group, R⁴² is a C₁-C₅ alkylene group or a hydroxy substituted C₁-C₅ alkylene group, n is an integer from 2 to 6, A is a carboxylate or sulfonate group, and M is a salt forming cation. In one aspect, R⁴⁰ is a C₁₁-C₁₈ alkyl group or a C₁₁-C₁₈ alkenyl group. In one aspect, R⁴¹ is methyl. In one aspect, R⁴² is methylene, ethylene or hydroxy propylene. In one aspect, n is 3. In a further aspect, M is selected from sodium, potassium, magnesium, ammonium, and mono-, di- and triethanolamine cations.

Examples of suitable betaines include, but are not limited to, lauryl betaine, coco betaine, oleyl betaine, coco hexadecyl dimethylbetaine, coco dimethyl carboxymethyl betaine, lauryl dimethyl carboxymethyl betaine, cetyl dimethyl carboxymethyl betaine, lauryl amidopropyl betaine, cocoamidopropyl betaine (CAPB), coco dimethyl sulfopropyl betaine, stearyl dimethyl sulfopropyl betaine, lauryl dimethyl sulfoethyl betaine, and cocamidopropyl hydroxysultaine.

The alkylamphocarboxylates such as the alkylamphoacetates and alkylamphopropionates (mono- and disubstituted carboxylates) can be represented by the formula:

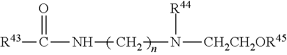

wherein R⁴³ is a C₇-C₂₂ alkyl or alkenyl group, R⁴⁴ is —CH₂C(O)O⁻M⁺, —CH₂CH₂C(O)O⁻M⁺, or —CH₂CH(OH)CH₂SO₃⁻M⁺, R⁴⁵ is hydrogen or —CH₂C(O)O⁻M⁺, and M is a cation selected from sodium, potassium, magnesium, ammonium, and the ammonium salt of mono-, di- and triethanolamine.

Exemplary alkylamphocarboxylates include, but are not limited to, sodium cocoamphoacetate, sodium lauroamphoacetate, sodium capryloamphoacetate, disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, and disodium capryloamphodipropionate.

The amount of such amphoteric or zwitterionic detersive surfactants ranges from about 0.5 to about 20 weight percent in one aspect, and from about 1 to about 10 weight percent in another aspect, based on the weight of the total composition.

Non-limiting examples of nonionic surfactants are disclosed in McCutcheon's Detergents and Emulsifiers, North American Edition, 1998, supra; and McCutcheon's, Functional Materials, North American, supra; both of which are incorporated by reference herein in their entirety. Additional Examples of nonionic surfactants are described in U.S. Pat. No. 4,285,841, to Barrat et al., and U.S. Pat. No. 4,284,532, to Leikhim et al., both of which are incorporated by reference herein in their entirety. Nonionic surfactants typically have a hydrophobic portion, such as a long chain alkyl group or an alkylated aryl group, and a hydrophilic portion containing various degrees of ethoxylation and/or propoxylation (e.g., 1 to about 50) ethoxy and/or propoxy moieties. Examples of some classes of nonionic surfactants that can be used include, but are not limited to, ethoxylated alkylphenols, ethoxylated and propoxylated fatty alcohols, polyethylene glycol ethers of methyl glucose, polyethylene glycol ethers of sorbitol, ethylene oxide-propylene oxide block copolymers, ethoxylated esters of fatty acids, condensation products of ethylene oxide with long chain amines or amides, condensation products of ethylene oxide with alcohols, and mixtures thereof.

Suitable nonionic surfactants include, for example, alkyl polysaccharides, alcohol ethoxylates, block copolymers, castor oil ethoxylates, ceto/oleyl alcohol ethoxylates, cetearyl alcohol ethoxylates, decyl alcohol ethoxylates, dinonyl phenol ethoxylates, dodecyl phenol ethoxylates, end-capped ethoxylates, ether amine derivatives, ethoxylated alkanolamides, ethylene glycol esters, fatty acid alkanolamides, fatty alcohol alkoxylates, lauryl alcohol ethoxylates, mono-branched alcohol ethoxylates, nonyl phenol ethoxylates, octyl phenol ethoxylates, oleyl amine ethoxylates, random copolymer alkoxylates, sorbitan ester ethoxylates, stearic acid ethoxylates, stearyl amine ethoxylates, tallow oil fatty acid ethoxylates, tallow amine ethoxylates, tridecanol ethoxylates, acetylenic diols, polyoxyethylene sorbitols, and mixtures thereof. Various specific examples of suitable nonionic surfactants include, but are not limited to, Cocamide MEA, Cocamide MIPA, methyl gluceth-10, PEG-20 methyl glucose distearate, PEG-20 methyl glucose sesquistearate, ceteth-8, ceteth-12, dodoxynol-12, laureth-15, PEG-20 castor oil, polysorbate 20, steareth-20, polyoxyethylene-10 cetyl ether, polyoxyethylene-10 stearyl ether, polyoxyethylene-20 cetyl ether, polyoxyethylene-10 oleyl ether, polyoxyethylene-20 oleyl ether, an ethoxylated nonylphenol, ethoxylated octylphenol, ethoxylated dodecylphenol, or ethoxylated fatty (C₆-C₂₂) alcohol, including 3 to 20 ethylene oxide moieties, polyoxyethylene-20 isohexadecyl ether, polyoxyethylene-23 glycerol laurate, polyoxyethylene-20 glyceryl stearate, PPG-10 methyl glucose ether, PPG-20 methyl glucose ether, polyoxyethylene-20 sorbitan monoesters, polyoxyethylene-80 castor oil, polyoxyethylene-15 tridecyl ether, polyoxyethylene-6 tridecyl ether, laureth-2, laureth-3, laureth-4, PEG-3 castor oil, PEG 600 dioleate, PEG 400 dioleate, poloxamers such as poloxamer 188, polysorbate 21, polysorbate 40, polysorbate 60, polysorbate 61, polysorbate 65, polysorbate 80, polysorbate 81, polysorbate 85, sorbitan caprylate, sorbitan cocoate, sorbitan diisostearate, sorbitan dioleate, sorbitan distearate, sorbitan fatty acid ester, sorbitan isostearate, sorbitan laurate, sorbitan oleate, sorbitan palmitate, sorbitan sesquiisostearate, sorbitan sesquioleate, sorbitan sesquistearate, sorbitan stearate, sorbitan triisostearate, sorbitan trioleate, sorbitan tristearate, sorbitan undecylenate, or mixtures thereof.

Alkyl glucoside nonionic surfactants can also be employed and are generally prepared by reacting a polysaccharide or monosaccharide with an alcohol such as a fatty alcohol in an acid medium. For example, U.S. Pat. Nos. 5,527,892 and 5,770,543 describe alkyl glucosides and/or methods for their preparation. Suitable examples are commercially available under the names of Glucopon™ 220, 225, 425, 600 and 625, PLANTACARE®, and PLANTAPON®, all of which are available from Cognis Corporation.

In another aspect, nonionic surfactants include, but are not limited to, alkoxylated methyl glucosides such as, for example, methyl gluceth-10, methyl gluceth-20, PPG-10 methyl glucose ether, and PPG-20 methyl glucose ether, available from Lubrizol Advanced Materials, Inc., under the trade names, Glucam®E10, Glucam® E20, Glucam® P10, and Glucam® P20, respectively; and hydrophobically modified alkoxylated methyl glucosides, such as PEG 120 methyl glucose dioleate, PEG-120 methyl glucose trioleate, and PEG-20 methyl glucose sesquistearate, available from Lubrizol Advanced Materials, Inc., under the trade names, Glucamate® DOE-120, Glucamate™ LT, and Glucamate™ SSE-20, respectively, are also suitable. Other exemplary hydrophobically modified alkoxylated methyl glucosides are disclosed in U.S. Pat. Nos. 6,573,375 and 6,727,357, the relevant disclosure of which are hereby incorporated by reference.

Other useful nonionic surfactants include water soluble silicones such as PEG-10 Dimethicone, PEG-12 Dimethicone, PEG-14 Dimethicone, PEG-17 Dimethicone, PPG-12 Dimethicone, PPG-17 Dimethicone and derivatized/functionalized forms thereof such as Bis-PEG/PPG-20/20 Dimethicone Bis-PEG/PPG-16/16 PEG/PPG-16/16 Dimethicone, PEG/PPG-14/4 Dimethicone, PEG/PPG-20/20 Dimethicone, PEG/PPG-20/23 Dimethicone, and Perfluorononylethyl Carboxydecyl PEG-10 Dimethicone.

In one embodiment, the amount of nonionic surfactant ranges from about 1 weight percent to about 40 weight percent in one aspect, from about 2.5 weight percent to about 35 weight percent in another aspect, from about 5 weight percent to about 30 weight percent in a further aspect, from about 10 weight percent to about 25 weight percent in a still further aspect, and from about 15 weight percent to about 22.5 weight percent in an additional aspect. Here, as well as elsewhere in the specification and claims, individual numerical values, or limits, can be combined to form additional non-disclosed and/or non-stated ranges. In another embodiment, when two or more different surfactants and/or different types of surfactants are utilized, the ratio of any two or more surfactants and/or types of surfactants can be any ratio typically used in home care, personal care, health care, home care, and/or I&I as known to those of skill in the art.

In one embodiment of the disclosed technology, at least one anionic surfactant is utilized in combination with an amphoteric or zwitterionic surfactant. In one aspect, the weight ratio (based on active material) of anionic surfactant (non-ethoxylated and/or ethoxylated) to amphoteric surfactant can range from about 10:1 to about 2:1 in one aspect, and can be about 9:1, about 8:1, about 7:1, about 6:1, about 5:1, about 4.5:1, about 4:1, or about 3:1 in another aspect. When employing an ethoxylated anionic surfactant in combination with a non-ethoxylated anionic surfactant and an amphoteric or zwitterionic surfactant, the weight ratio (based on active material) of ethoxylated anionic surfactant to non-ethoxylated anionic surfactant to amphoteric surfactant can range from about 3.5:3.5:1 in one aspect to about 1:1:1 in another aspect.

In one aspect, the anionic surfactant is selected from alkyl sulfates, including sodium lauryl sulfate, ammonium lauryl sulfate, sodium coco-sulfate, and mixtures thereof.

In one aspect, the anionic surfactant is selected from ethoxylated alkyl sulfates including sodium laureth sulfate, ammonium laureth sulfate, sodium trideceth sulfate, and mixtures thereof.

In one aspect, the anionic surfactant is selected from amino acid based surfactants, isethionate based surfactants, sulfosuccinate based surfactants, and alkyl sulfoacetates.

In one aspect, the optional amphoteric surfactant is selected from alkyl betaines, amidoalkyl betaines and amidoalkyl sultaines including lauryl betaine, cocamidopropyl betaine, cocamidopropyl hydroxysultaine, and mixtures thereof.

The sulfate-free surfactants suitable for use in the present technology are any of the sulfate-free anionic, cationic, amino acid, amphoteric and nonionic surfactants mentioned above. Exemplary anionic sulfate-free surfactants are selected from disodium laureth sulfosuccinate, sodium lauroyl methyl isethionate, sodium cocoyl isethionate, sodium C₁₄-C₁₆ alpha-olefin sulfonate, sodium lauryl sulfoacetate, sodium methyl cocoyl taurate, sodium lauroyl sarcosinate. Exemplary sulfate-free amino acid surfactants include sodium cocoyl alaninate, sodium cocoyl glycinate, and disodium cocoyl glutamate. Exemplary sulfate-free amphoteric surfactants are cocamidopropyl betaine and sodium cocoamphoacetate. An exemplary sulfate-free nonionic surfactant is coco-glucoside. Mixtures of one or more of the foregoing sulfate-free surfactants are also contemplated in combination with the HASE polymers of the disclosed technology.

Aqueous Carrier

The compositions of the present technology are typically in the form of pourable liquids (under ambient conditions). The compositions will therefore typically comprise an aqueous carrier, which is present at a level of from about 20 wt. % to about 95 wt. % in one aspect, and from about 60 wt. % to about 85 wt. % in another aspect, based on the weight of the total composition. The aqueous carrier may comprise water, or a miscible mixture of water and organic solvent, but preferably comprises water with minimal or no significant concentrations of organic solvent, except as otherwise incidentally incorporated into the composition as minor ingredients of other essential or optional components.

E. Optional Components

The compositions of the present technology may further comprise one or more optional components known for use in hair care or personal care products, provided that the optional components are physically and chemically compatible with the essential components described herein, or do not otherwise unduly impair product stability, aesthetics or performance. Unless otherwise stated individual concentrations of such optional components may range from about 0.001 wt. % to about 20 wt. %, based on the weight of the total composition.

Non-limiting examples of optional components for use in the composition include insoluble or particulate materials, conditioning agents (silicones, hydrocarbon oils, fatty esters,), auxiliary viscosity modifiers, humectants, sensates, botanicals, amino acids, vitamins, chelating agents, buffering agents, pH adjusting agents, preservatives perfumes and fragrances, electrolytes, dyes and pigments, nonvolatile solvents or diluents (water soluble and insoluble), foam boosters, sunscreens and UV absorbers.

Insoluble and Particulate Materials

In the compositions of the present technology, the HASE polymers of the disclosed technology can be utilized to enhance foaming properties, improve mildness and the rheology properties of cleansing compositions for the hair, scalp and skin, and can be utilized for the stable suspension of insoluble silicones, opacifiers and pearlescent agents (e.g., mica, coated mica, ethylene glycol monostearate (EGMS), ethylene glycol distearate (EGDS), polyethylene glycol monostearate (PGMS) or polyethyleneglycol distearate (PGDS)), pigments, exfoliants, auxiliary anti-dandruff agents, clay, swellable clay, laponite, gas bubbles, liposomes, microsponges, cosmetic beads, cosmetic microcapsules, and flakes, and are discussed in more detail below.

Exemplary cosmetic bead components include, but are not limited to, agar beads, alginate beads, jojoba beads, gelatin beads, Styrofoam™ beads, polyacrylate, polymethylmethacrylate (PMMA), polyethylene beads, Unispheres™ and Unipearls™ cosmetic beads (Induchem USA, Inc., New York, N.Y.), Lipocapsule™, Liposphere™, and Lipopearl™ microcapsules (Lipo Technologies Inc., Vandalia, Ohio), and Confetti II™ dermal delivery flakes (United-Guardian, Inc., Hauppauge, N.Y.). Beads can be utilized as aesthetic materials or can be used to encapsulate benefit agents to protect them from the deteriorating effects of the environment or for optimal delivery, release and performance in the final product.

In one aspect, the cosmetic beads range in size from about 0.5 to about 1.5 mm. In another aspect, the difference in specific gravity of the bead and water is between about +/−0.01 and 0.5 in one aspect, and from about +/−0.2 to 0.3 g/ml in another aspect.

In one aspect, the microcapsules range in size from about 0.5 to about 300 μm. In another aspect, the difference in specific gravity between the microcapsules and water is from about +/−0.01 to 0.5. Non-limiting examples of microcapsule beads are disclosed in U.S. Pat. No. 7,786,027, the disclosure of which is herein incorporated by reference.

Conditioning Agents

Conditioning agents include any material which is used to give a particular conditioning benefit to hair, scalp and/or skin. In hair treatment compositions, suitable conditioning agents are those which deliver one or more benefits relating to shine, softness, combability, antistatic properties, wet-handling, damage, manageability, body, and greasiness. The conditioning agents useful in the compositions of the present technology typically comprise a water insoluble, water dispersible, non-volatile, liquid that forms emulsified, liquid particles. Suitable conditioning agents for use in the composition are those conditioning agents characterized generally as silicones (e.g., silicone oils, cationic silicones, silicone gums, high refractive silicones, and silicone resins), organic conditioning oils (e.g., hydrocarbon oils, polyolefins, and fatty esters) or combinations thereof, or those conditioning agents which otherwise form liquid, dispersed particles in the aqueous surfactant matrix herein. Such conditioning agents should be physically and chemically compatible with the essential components of the composition, and should not otherwise unduly impair product stability, aesthetics or performance.

Silicones

The silicone conditioning agent may comprise volatile silicones, non-volatile silicones, and mixtures thereof. If volatile silicones are present, they are typically employed as a solvent or carrier for commercially available forms of non-volatile silicone fluid conditioning agents such as oils and gums and resins. Volatile silicone fluids are often included in the conditioning package to improve silicone fluid deposition efficacy or to enhance the shine, sheen or glossiness of the hair. Volatile silicone materials are frequently included in formulations to enhance sensory attributes (e.g., feel) on the hair, scalp and skin

In one aspect, the silicone conditioning agent is non-volatile and includes silicone oils, gums, resins and mixtures thereof. By non-volatile is meant that the silicone has a very low vapor pressure at ambient temperature conditions (e.g., less than 2 mm Hg at 20° C.). The non-volatile silicone conditioning agent has a boiling point above about 250° C. in one aspect, above about 260° C. in another aspect, and above about 275° C. in a further aspect. Background information on silicones including sections discussing silicone oils, gums, and resins, as well as their manufacture, are found in Encyclopedia of Polymer Science and Engineering, vol. 15, 2d ed., pp 204-308, John Wiley & Sons, Inc. (1989).

Silicone Oil

In one aspect, the silicone conditioning agent is silicone oil selected from a polyorganosiloxane material. In one aspect, the polyorganosiloxane material can be selected from polyalkylsiloxanes, polyarylsiloxanes, polyalkylarylsiloxanes, hydroxyl terminated polyalkylsiloxanes, polyarylalkylsiloxanes, amino functional polyalkylsiloxanes, quaternary functional polyalkylsiloxanes, and mixtures thereof.

In one aspect, the silicone oil conditioning agent includes polyorganosiloxanes represented by the formula:

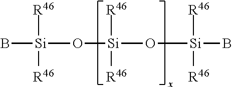

wherein B independently represents hydroxy, methyl, methoxy, ethoxy, propoxy, and phenoxy; R⁴⁶ independently represents methyl, ethyl, propyl, phenyl, methylphenyl, phenylmethyl, a primary, secondary or tertiary amine, a quaternary group selected from a group selected from:
—R⁴⁷—N(R⁴⁸)CH₂CH₂N(R⁴⁸)₂;
—R⁴⁷—N(R⁴⁸)₂;
—R⁴⁷—N+(R⁴⁸)₃CA⁻; and
—R⁴⁷—N(R⁴⁸)CH₂CH₂N+(R⁴⁸)H₂ CA⁻;
   wherein R⁴⁷ is a linear or branched, hydroxyl substituted or unsubstituted alkylene or alkylene ether moiety containing 2 to 10 carbon atoms; R⁴⁸ is hydrogen, C₁-C₂₀ alkyl (e.g, methyl), phenyl or benzyl; q is an integer ranging from about 2 to about 8; CA⁻ is a halide ion selected from chlorine, bromine, iodine and fluorine; and x is an integer ranging from about 7 to about 8000 in one aspect, from about 50 to about 5000 in another aspect, form about 100 to about 3000 in still another aspect, and from about 200 to about 1000 in a further aspect.

In one aspect, the amino functional polyalkylsiloxane can be represented by the formula:

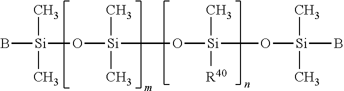

wherein B independently represents hydroxy, methyl, methoxy, ethoxy, propoxy, and phenoxy; and R⁴⁰ is selected from:
—R⁴⁷—N(R⁴⁸)CH₂CH₂N(R⁴⁸)₂;
—R⁴⁷—N(R⁴⁸)₂;
—R⁴⁷—N+(R⁴⁸)₃CA⁻; and
—R⁴⁷—N(R⁴⁸)CH₂CH₂N(R⁴⁸)H₂ CA⁻
   wherein R⁴⁷ is a linear or branched, hydroxyl substituted or unsubstituted alkylene or alkylene ether moiety containing 2 to 10 carbon atoms; R⁴⁸ is hydrogen, C₁-C₂₀ alkyl (e.g, methyl), phenyl or benzyl; CA⁻ is a halide ion selected from chlorine, bromine, iodine and fluorine; and the sum of m+n ranges from about 7 to about 1000 in one aspect, from about 50 to about 250 in another aspect, and from about 100 to about 200 in another aspect, subject to the proviso that m or n is not 0. In one aspect B is hydroxy and R⁴⁶ is —(CH₂)₃NH(CH₂)₃NH₂. In another aspect B is methyl and R⁴⁶ is —(CH₂)₃NH(CH₂)₃NH₂. In still another aspect B is methyl and R⁴⁶ is a quaternary ammonium moiety represented by —(CH₂)₃OCH₂CH(OH)CH₂N⁺(R⁴⁸)₃ CA⁻; wherein R⁴⁸ and CA⁻ are as previously defined.

The silicone oil conditioning agents can have a viscosity ranging from about above about 25 to about 1,000,000 mPa·s at 25° C. in one aspect, from about 100 to about 600,000 mPa·s in another aspect, and from about 1000 to about 100,000 mPa·s still another aspect, from about 2,000 to about 50,000 mPa·s in yet another aspect, and from about 4,000 to about 40,000 mPa·s in a further aspect. The viscosity is measured by means of a glass capillary viscometer as described by Dow Corning Corporate Test Method CTM004, dated Jul. 20, 1970. In one aspect the silicone oils have an average molecular weight below about 200,000 daltons. The average molecular weight can typically range from about 400 to about 199,000 daltons in one aspect, from about 500 to about 150,000 daltons in another aspect, from about 1,000 to about 100,000 daltons in still another aspect, from about 5,000 to about 65,000 daltons in a further aspect.

Exemplary silicone oil conditioning agents include, but are not limited to, polydimethylsiloxanes (dimethicones), polydiethylsiloxanes, polydimethyl siloxanes having terminal hydroxyl groups (dimethiconols), polymethylphenylsiloxanes, phenylmethylsiloxanes, amino functional polydimethylsiloxanes (amodimethicones), and mixtures thereof.

Silicone Gum

Another silicone conditioning agent useful in the disclosed technology is a silicone gum. A silicone gum is a polyorganosiloxane material of the same general structure of the silicone oils set forth above wherein B independently represents hydroxy, methyl, methoxy, ethoxy, propoxy, and phenoxy; R⁴⁶ independently represents methyl, ethyl, propyl, phenyl, methylphenyl, phenylmethyl, and vinyl. Silicone gums have a viscosity measured at 25° C. of greater than 1,000,000 mPa·s. The viscosity can be measured by means of a glass capillary viscometer as described above for the silicone oils. In one aspect the silicone gums have an average molecular weight about 200,000 daltons and above. The molecular weight can typically range from about 200,000 to about 1,000,000 daltons. It is recognized that the silicone gums described herein can also have some overlap with the silicone oils described previously. This overlap is not intended as a limitation on any of these materials.

Suitable silicone gums for use in the silicone component of compositions of the disclosed technology are polydimethylsiloxanes (dimethicones), optionally having terminal end groups such as hydroxyl (dimethiconols), polymethylvinylsiloxane, polydiphenylsiloxane, and mixtures thereof.

Silicone Resins

Silicone resins can be included as a silicone conditioning agent suitable for use in the compositions of the disclosed technology. These resins are crosslinked polysiloxanes. The crosslinking is introduced through the incorporation of trifunctional and tetrafunctional silanes with monofunctional and/or difunctional silanes during manufacture of the silicone resin. As is well understood in the art, the degree of crosslinking that is required in order to result in a silicone resin will vary according to the specific silane units incorporated into the silicone resin. In general, silicone materials which have a sufficient level of trifunctional and tetra-functional siloxane monomer units (and hence, a sufficient level of crosslinking) such that they form a rigid or hard film are considered to be silicone resins. The ratio of oxygen atoms to silicon atoms is indicative of the level of crosslinking in a particular silicone material. Silicone materials which have at least about 1.1 oxygen atoms per silicon atom will generally be silicone resins herein. In one aspect, the ratio of oxygen:silicon atoms is at least about 1.2:1.0. Silanes used in the manufacture of silicone resins include monomethyl-, dimethyl-, trimethyl-, monophenyl-, diphenyl-, methylphenyl-, monovinyl-, and methylvinyl-chlorosilanes, and terachlorosilane, with the methyl substituted silanes being most commonly utilized.

Silicone materials and silicone resins can be identified according to a shorthand nomenclature system known to those of ordinary skill in the art as “MDTQ” nomenclature. Under this naming system, the silicone is described according to the presence of various siloxane monomer units which make up the silicone. The “MDTQ” nomenclature system is described in the publication entitled “Silicones: Preparation, Properties and Performance”; Dow Corning Corporation, 2005, and in U.S. Pat. No. 6,200,554.

Exemplary silicone resins for use in the compositions of the disclosed technology include, but are not limited to MQ, MT, MTQ, MDT and MDTQ resins. In one aspect, methyl is the silicone resin substituent. In another aspect, the silicone resin is selected from a MQ resins, wherein the M:Q ratio is from about 0.5:1.0 to about 1.5:1.0 and the average molecular weight of the silicone resin is from about 1000 to about 10,000 daltons.

Volatile Silicones

The optional volatile silicones referred to above include linear polydimethylsiloxanes and cyclic polydimethylsiloxanes (cyclomethicones), and mixtures thereof. Volatile linear polydimethylsiloxanes (dimethicones) typically contain about 2 to about 9 silicon atoms, alternating with oxygen atoms in a linear arrangement. Each silicon atom is also substituted with two alkyl groups (the terminal silicon atoms are substituted with three alkyl groups), such as, for example, methyl groups. The cyclomethicones typically contain about 3 to about 7 dimethyl substituted silicon atoms in one aspect and from about 3 to about 5 in another aspect, alternating with oxygen atoms, in a cyclic ring structure. The term “volatile” means that the silicone has a measurable vapor pressure, or a vapor pressure of at least 2 mm of Hg at 20° C. The volatile silicones have a viscosity of 25 mPa·s or less at 25° C. in one aspect, from about 0.65 about to about 10 mPa·s in another aspect, from about 1 to about 5 mPa·s in still another aspect, and from about 1.5 to about 3.5 mPa·s in a further aspect. A description of linear and cyclic volatile silicones is found in Todd and Byers, “Volatile Silicone Fluids for Cosmetics”, Cosmetics and Toiletries, Vol. 91(1), pp. 27-32 (1976), and in Kasprzak, “Volatile Silicones”, Soap/Cosmetics/Chemical Specialities, pp. 40-43 (December 1986).

Exemplary volatile linear dimethicones include, but are not limited to, hexamethyldisiloxane, octamethyltrisiloxane, decamethyltetrasiloxane, dodecamethylpentasiloxane and blends thereof. Volatile linear dimethicones and dimethicone blends are commercially available from Dow Corning Corporation as Dow Corning 200® Fluid (e.g., product designations 0.65 CST, 1 CST, 1.5 CST, and 2 CST) and Dow Corning® 2-1184 Fluid.

Exemplary volatile cyclomethicones are D4 cyclomethicone (octamethylcyclotetrasiloxane), D5 cyclomethicone (decamethylcyclopentasiloxane), D6 cyclomethicone, and blends thereof (e.g., D4/D5 and D5/D6). Volatile cyclomethicones and cyclomethicone blends are commercially available from Momentive Performance Materials Inc. as SF1173, SF1202, SF1256, and SF1258 silicone fluids, and Dow Corning Corporation as Dow Corning® 244, 245, 246, 345, and 1401 silicone fluids. Blends of volatile cyclomethicones and volatile linear dimethicones also can be employed.

The amount of silicone conditioner(s) in the compositions of the present technology should be sufficient to provide the desired conditioning performance to the hair, and generally ranges from about 0.01 to about 20 wt. % in one aspect, from about 0.05 to about 15 wt. % in another aspect, from about 0.1% to about 10 wt. % in still another aspect, and from about 1 to about 5 wt. % in a further aspect, based on the total weight of the composition.

Hydrocarbon Oils

The conditioning component of the compositions of the disclosed technology can also contain hydrocarbon oil conditioners.

Suitable conditioning oils for use as conditioning agents in the compositions of the disclosed technology include, but are not limited to, hydrocarbon oils having at least about 10 carbon atoms, such as cyclic hydrocarbons, straight chain aliphatic hydrocarbons (saturated or unsaturated), and branched chain aliphatic hydrocarbons (saturated or unsaturated), including polymers and mixtures thereof. Straight chain hydrocarbon oils typically contain about 12 to 19 carbon atoms. Branched chain hydrocarbon oils, including hydrocarbon polymers, typically will contain more than 19 carbon atoms.

Specific non-limiting examples of these hydrocarbon oils include paraffin oil, mineral oil, saturated and unsaturated dodecane, saturated and unsaturated tridecane, saturated and unsaturated tetradecane, saturated and unsaturated pentadecane, saturated and unsaturated hexadecane, polybutene, polydecene, and mixtures thereof. Branched-chain isomers of these compounds, as well as of higher chain length hydrocarbons, can also be used, examples of which include highly branched, saturated or unsaturated, alkanes such as the permethyl-substituted isomers, e.g., the permethyl-substituted isomers of hexadecane and eicosane, such as 2,2,4,4,6,6,8,8-dimethyl-10-methylundecane and 2,2,4,4,6,6-dimethyl-8-methylnonane, available from Permethyl Corporation. Hydrocarbon polymers such as polybutene and polydecene. A preferred hydrocarbon polymer is polybutene, such as the copolymer of isobutylene and butene. A commercially available material of this type is L-14 polybutene from BP Chemical Company.

Liquid polyolefin conditioning oils can be used in the hair straightening compositions of the present technology. The liquid polyolefin conditioning agents are typically poly-α-olefins that have been hydrogenated. Polyolefins for use herein can be prepared by the polymerization of C₄ to about C₁₄ olefinic monomers. Non-limiting examples of olefinic monomers for use in preparing the polyolefin liquids herein include ethylene, propylene, 1-butene, 1-pentene, 1-hexene, 1-octene, 1-decene, 1-dodecene, 1-tetradecene, branched chain isomers such as 4-methyl-1-pentene, and mixtures thereof. In one aspect of the disclosed technology, hydrogenated α-olefin monomers include, but are not limited to: 1-hexene to 1-hexadecenes, 1-octene to 1-tetradecene, and mixtures thereof.

Fluorinated or perfluorinated oils are also contemplated within the scope of the present technology. Fluorinated oils include perfluoropolyethers described in European Patent 0 486 135 and the fluorohydrocarbon compounds described in WO 93/11103. The fluoridated oils may also be fluorocarbons such as fluoramines, e.g., perfluorotributylamine, fluoridated hydrocarbons, such as perfluorodecahydronaphthalene, fluoroesters, and fluoroethers.

Natural Oils

Natural oil conditioners are also useful in the practice of the disclosed technology and include but are not limited to peanut, sesame, avocado, coconut, cocoa butter, almond, safflower, corn, cotton seed, sesame seed, walnut oil, castor, olive, jojoba, palm, palm kernel, soybean, wheat germ, linseed, sunflower seed; eucalyptus, lavender, vetiver, litsea, cubeba, lemon, sandalwood, rosemary, chamomile, savory, nutmeg, cinnamon, hyssop, caraway, orange, geranium, cade, and bergamot oils, fish oils, glycerol tricaprocaprylate; and mixtures thereof.

Ester Oils

Ester oil conditioners include, but are not limited to, fatty esters having at least 10 carbon atoms. These fatty esters include esters derived from fatty acids or alcohols (e.g., mono-esters, polyhydric alcohol esters, and di- and tri-carboxylic acid esters). The fatty esters hereof may include or have covalently bonded thereto other compatible functionalities, such as amides and alkoxy moieties (e.g., ethoxy or ether linkages, etc.).

Exemplary fatty esters include, but are not limited to isopropyl isostearate, hexyl laurate, isohexyl laurate, isohexyl palmitate, isopropyl palmitate, decyl oleate, isodecyl oleate, hexadecyl stearate, decyl stearate, isopropyl isostearate, dihexyldecyl adipate, lauryl lactate, myristyl lactate, cetyl lactate, oleyl stearate, oleyl oleate, oleyl myristate, lauryl acetate, cetyl propionate, and oleyl adipate.

Other fatty esters suitable for use in the compositions of the disclosed technology are mono-carboxylic acid esters of the general formula R⁶⁰C(O)OR⁶¹, wherein R⁶⁰ and R⁶¹ are alkyl or alkenyl radicals, and the sum of carbon atoms in R⁶⁰ and R⁶¹ is at least 10 in one aspect, and at least 22 in another aspect of the disclosed technology.

Still other fatty esters suitable for use in the compositions of the disclosed technology are di- and tri-alkyl and alkenyl esters of carboxylic acids, such as esters of C₄ to C₈ dicarboxylic acids (e.g., C₁ to C₂₂ esters, preferably C₁ to C₆, of succinic acid, glutaric acid, adipic acid). Specific non-limiting examples of di- and tri-alkyl and alkenyl esters of carboxylic acids include isocetyl stearyol stearate, diisopropyl adipate, and tristearyl citrate.

Other fatty esters suitable for use in the compositions of the disclosed technology are those known as polyhydric alcohol esters. Such polyhydric alcohol esters include alkylene glycol esters, such as ethylene glycol mono and di-fatty acid esters, diethylene glycol mono- and di-fatty acid esters, polyethylene glycol mono- and di-fatty acid esters, propylene glycol mono- and di-fatty acid esters, polypropylene glycol monooleate, polypropylene glycol 2000 monostearate, ethoxylated propylene glycol monostearate, glyceryl mono- and di-fatty acid esters, polyglycerol poly-fatty acid esters, ethoxylated glyceryl monostearate, 1,3-butylene glycol monostearate, 1,3-butylene glycol distearate, polyoxyethylene polyol fatty acid ester, sorbitan fatty acid esters, and polyoxyethylene sorbitan fatty acid esters.

Specific non-limiting examples of suitable synthetic fatty esters include: P-43 (C₈ to C₁₀ triester of trimethylolpropane), MCP-684 (tetraester of 3,3 diethanol-1,5 pentadiol), MCP 121 (C₈ to C₁₀ diester of adipic acid), all of which are available from ExxonMobil Chemical Company.

The amount of hydrocarbon and natural conditioning oils and ester oil conditioning agents can range from about 0.05 to about 10 wt. %, in one aspect, from about 0.5 to about 5 wt. % in another aspect, and from about 1 to about 3 wt. % in a further aspect, based on the total weight of the composition.

Cationic Compounds and Polymers

Cationic Compounds refer to non-polymeric and polymeric compounds containing at least one cationic moiety or at least one moiety that can be ionized to form a cationic moiety. Typically these cationic moieties are nitrogen containing groups such as quaternary ammonium or protonated amino groups. The cationic protonated amines can be primary, secondary, or tertiary amines. In one aspect, the cationic conditioning compounds include quaternary nitrogen containing non-polymeric and polymeric materials that well known in the art for hair conditioning. Cationic conditioning compounds include non-polymeric compounds containing one quaternary ammonium salt moiety and polymeric compounds (polymers) containing at least one quaternary ammonium salt moiety.

In one aspect, the quaternary ammonium salt moiety corresponds to the general formula: (R⁷⁰)(R⁷¹)(R⁷²)(R⁷³)N⁺) E⁻ where each of R⁷⁰, R⁷¹, R⁷⁴, and R⁷⁵ are independently selected from an aliphatic group having from 1 to about 22 carbon atoms (e.g., alkyl, alkenyl); an aromatic (e.g., phenyl benzyl); alkoxy; polyoxyalkylene (e.g., polyethylene, polypropylene, and combinations thereof); acetamido; alkylamido; alkylamidoalkyl; hydroxyalkyl; aryl; araalkyl; or alkylaryl group having 1 to about 22 carbon atoms in the alkyl chain; and E⁻ is a salt-forming anion such as those selected from halogen, (e.g., chloride, bromide), acetate, citrate, lactate, glycolate, phosphate, nitrate, sulfate, and alkylsulfate (e.g., methosulfate, ethosulfate). The aliphatic groups can contain, in addition to carbon and hydrogen atoms, ether linkages, ester linkages, and other groups such as amino groups. The longer chain aliphatic groups, e.g., those of about 12 carbons, or higher, can be saturated or unsaturated. Any two of R⁷⁰, R⁷¹, R⁷⁴, and R⁷⁵ together with the nitrogen atom to which they are attached can be taken together to form a ring structure containing 5 to 6 carbon atoms, one of said carbon atoms can optionally be replaced with a heteroatom selected from nitrogen, oxygen or sulfur.

In one aspect, the quaternary ammonium moiety contains at least one nitrogen atom that is covalently linked to at least three alkyl and/or aryl substituents, and the nitrogen atom remains positively charged regardless of the environmental pH.

In one aspect, the quaternary ammonium moiety contains one nitrogen atom and at least one C₁₂ to C₂₂ alkyl group. In one aspect, the quaternary ammonium moiety contains one C₁₂ to C₂₂ alkyl group and at least two C₁ to C₅ alkyl groups (e.g., methyl, ethyl, propyl, butyl and pentyl, and combinations thereof). In one aspect, the quaternary ammonium moiety contains one C₁₂ to C₂₂ alkyl group, and three C₁ to C₅ alkyl groups (e.g., methyl, ethyl, propyl, butyl and pentyl, and combinations thereof). In one aspect, the quaternary ammonium moiety contains one C₁₂ to C₂₂ alkyl group, and two C₁ to C₅ alkyl groups (e.g., methyl, ethyl, propyl, butyl and pentyl, and combinations thereof), and one moiety containing an alkoxy; polyoxyalkylene (e.g., polyethylene, polypropylene, and combinations thereof), where the polyoxyalkylene moiety contains 3 to 100 repeating units; acetamide; alkylamido; alkylamidoalkyl; hydroxyalkyl; aryl; araalkyl; or alkylaryl group having 1 to about 22 carbon atoms in the alkyl chain, and having 6 to about 14 carbon atoms in the aryl moiety.

A number of quaternary nitrogen-containing compounds and polymers, their manufacturers and general descriptions of their chemical characteristics are found in the CTFA Dictionary and in the International Cosmetic Ingredient Dictionary, Vol. 1 and 2, 5th Ed., published by the Cosmetic Toiletry and Fragrance Association, Inc. (CTFA) (1993), the pertinent disclosures of which are incorporated herein by reference. The name assigned to the ingredients by the CTFA or by the manufacturer is used for convenience.

Non-limiting examples of monomeric quaternary ammonium compounds useful as cationic conditioners in the present technology include Acetamidopropyl Trimonium Chloride, Behenamidopropyl Ethyldimonium Ethosulfate, Behentrimonium Chloride, Behentrimonium Methosulfate, Cetethyl Morpholinium Ethosulfate, Cetrimonium Chloride, Cocoamidopropyl Ethyldimonium Ethosulfate, Dicetyldimonium Chloride, Hydroxyethyl Behenamidopropyl Dimonium Chloride, Quaternium-26, Quaternium-27, Quaternium-53, Quaternium-63, Quaternium-70, Quaternium-72, Quaternium-76 PPG-9 Diethylmonium Chloride, PPG-25 Diethylmonium Chloride, PPG-40 Stearalkonium Chloride, Isostearamidopropyl Ethyldimonium Ethosulfate, and mixtures thereof.

Cationic polymers are also useful as conditioning agents alone or in combination with the other conditioning agents described herein. Suitable cationic polymers can be synthetically derived or natural polymers can be synthetically modified to contain cationic moieties. Polymeric quaternary ammonium moiety salt containing polymers can be prepared by the polymerization of a diallylamine such as dialkyldiallylammonium salt or copolymer thereof in which the alkyl group contains 1 to about 22 carbon atoms in one aspect and methyl or ethyl in another aspect. Copolymers containing a quaternary moiety derived from a dialkyldiallylammonium salt and an anionic component derived from anionic monomers of acrylic acid and methacrylic acid are suitable conditioning agents. Also suitable are, polyampholyte terpolymers having a cationic component prepared from a derivative of diallylamine, such as a dimethyldiallylammonium salt, an anionic component derived from anionic monomers of acrylic acid or 2-acrylamido-2-methylpropane sulfonic acid and a nonionic component derived from nonionic monomers of acrylamide. The preparation of such quaternary ammonium salt moiety containing polymers can be found, for example, in U.S. Pat. Nos. 3,288,770; 3,412,019; 4,772,462 and 5,275,809, the pertinent disclosures of which are incorporated herein by reference.

In one aspect, suitable cationic polymers include the chloride salts of the foregoing quaternized homopolymers and copolymers in which the alkyl group is methyl or ethyl, and are commercially available under the Merquat® series of trademarks from Lubrizol Advanced Materials, Inc.

A homopolymer prepared from diallyl dimethyl ammonium chloride (DADMAC) having the CTFA name, Polyquaternium-6, is available under the Merquat 100 and Merquat 106 trademark. A copolymer prepared from DADMAC and acrylamide having the CTFA name, Polyquaternium-7, is sold under the Merquat 550 trademark. Another copolymer prepared from DADMAC and acrylic acid having the CTFA name, Polyquaternium-22, is sold under the Merquat 280 trademark. The preparation of Polyquaternium-22 and its related polymers is described in U.S. Pat. No. 4,772,462, the pertinent disclosures of which are incorporated herein by reference.

Also useful is an ampholytic terpolymer prepared from a nonionic component derived from acrylamide or methyl acrylate, a cationic component derived from DADMAC or methacrylamidopropyl trimethyl ammonium chloride (MAPTAC), and an anionic component derived from acrylic acid or 2-acrylamido-2-methylpropane sulfonic acid or combinations of acrylic acid and 2-acrylamido-2-methylpropane sulfonic acid. An ampholytic terpolymer prepared from acrylic acid, DADMAC and acrylamide having the CTFA name, Polyquarternium-39, is available under the Merquat Plus 3330 trademark. Another ampholytic terpolymer prepared from acrylic acid, methacrylamidopropyl trimethyl ammonium chloride (MAPTAC) and methyl acrylate having the CTFA name, Polyquarternium-47, is available under the Merquat 2001 trademark. Still another ampholytic terpolymer prepared from acrylic acid, MAPTAC and acrylamide having the CTFA name, Polyquarternium-53, is available under the Merquat 2003PR trademark. The preparation of such terpolymers is described in U.S. Pat. No. 5,275,809, the pertinent disclosures of which are incorporated herein by reference.

Other cationic polymers and copolymers suitable as conditioners in the hair straightening compositions of the disclosed technology have the CTFA names Polyquaternium-4, Polyquaternium-11, Polyquarternium-16, Polyquaternium-28, Polyquaternium-29, Polyquaternium-32, Polyquaternium-33, Polyquaternium-35, Polyquaternium-37, Polyquaternium-44, Polyquaternium-46, Polyquaternium-47, Polyquaternium-52, Polyquaternium-53, Polyquarternium-55, Polyquaternium-59, Polyquaternium-61, Polyquaternium-64, Polyquaternium-65, Polyquaternium-67, Polyquaternium-69, Polyquaternium-70, Polyquaternium-71, Polyquaternium-72, Polyquaternium-73, Polyquaternium-74, Polyquaternium-76, Polyquaternium-77, Polyquaternium-78, Polyquaternium-79, Polyquaternium-80, Polyquaternium-81, Polyquaternium-82, Polyquaternium-84, Polyquaternium-85, Polyquaternium-87, and PEG-2-cocomonium chloride.

Exemplary cationically modified natural polymers suitable for use in the hair straightening composition include polysaccharide polymers, such as cationically modified cellulose and cationically modified starch derivatives modified with a quaternary ammonium halide moiety. Exemplary cationically modified cellulose polymers are salts of hydroxyethyl cellulose reacted with trimethyl ammonium substituted epoxide (CTFA, Polyquaternium-10). Other suitable types of cationically modified cellulose include the polymeric quaternary ammonium salts of hydroxyethyl cellulose reacted with lauryl dimethyl ammonium substituted epoxide (CTFA, Polyquaternium-24). Cationically modified potato starch having the CTFA name, Starch Hydroxypropyltrimonium Chloride, is available under the Sensomer™ CI-50 trademark, from Lubrizol Advanced Materials, Inc.

Other suitable cationically modified natural polymers include cationic polygalactomannan derivatives such as guar gum derivatives and cassia gum derivatives, e.g., CTFA: Guar Hydroxypropyltrimonium Chloride, Hydroxypropyl Guar Hydroxypropyltrimonium Chloride, and Cassia Hydroxypropyltrimonium Chloride. Guar hydroxypropyltrimonium chloride is commercially available under the Jaguar™ trade name series from Rhodia Inc. and the N-Hance trade name series from Ashland Inc. Cassia Hydroxypropyltrimonium Chloride is commercially available under the Sensomer™ CT-250 and Sensomer™ CT-400 trademarks from Lubrizol Advanced Materials, Inc.

The non-polymeric and polymeric cationic compounds can be present from about 0.05 to about 5 wt. % percent in one aspect, from about 0.1 to about 3 wt. percent in another aspect, and from about 0.5 to about 2.0 wt. % in a further aspect (based on the total weight of the composition).

Auxiliary Viscosity Modifier

The composition of the disclosed technology must be easily pourable with a shear thinning index of less than 0.5 at shear rates between 0.1 and 1 reciprocal second, and an optical transmission of at least 10%. The suspension agent of the disclosed technology optionally can be utilized in combination with an auxiliary rheology modifier (thickener) to enhance the yield value of a thickened liquid. In one aspect, the HASE polymer of the disclosed technology can be combined with a nonionic rheology modifier to enhance the yield stress value of a composition in which it is included. Any rheology modifier is suitable, so long as such is soluble in water, stable. Suitable rheology modifiers include, but are not limited to natural gums (e.g., polygalactomannan gums selected from fenugreek, cassia, locust bean, tara and guar), modified cellulose (e.g., ethylhexylethylcellulose (EHEC), hydroxybutylmethylcellulose (HBMC), hydroxyethylmethylcellulose (HEMC), hydroxypropylmethylcellulose (HPMC), methyl cellulose (MC), hydroxyethylcellulose (HEC), hydroxypropylcellulose (HPC) and cetyl hydroxyethylcellulose); and mixtures thereofmethylcellulose, polyethylene glycols (e.g., PEG 4000, PEG 6000, PEG 8000, PEG 10000, PEG 20000), polyvinyl alcohol, polyacrylamides (homopolymers and copolymers), and hydrophobically modified ethoxylated urethanes (HEUR). The rheology modifier can be utilized in an amount ranging from about 0.5 to about 25 wt. % in one aspect, from about 1 to about 15 wt. % in another aspect, and from about 2 to about 10 wt. % in a further aspect, based on the weight of the total weight of the composition.

Humectants

Humectants are defined as materials that absorb or release water vapor, depending on the relative humidity of the environment, (Harry's Cosmeticology, Chemical Publishing Company Inc., 1982 p. 266). Suitable humectants that include, but are not limited to, allantoin; pyrrolidonecarboxylic acid and its salts; hyaluronic acid and salts thereof; sorbic acid and salts thereof; urea, lysine, cystine, and amino acids; polyhydroxy alcohols such as glycerin, propylene glycol, hexylene glycol, hexanetriol, ethoxydiglycol, dimethicone copolyol, and sorbitol, and the esters thereof; polyethylene glycol; glycolic acid and glycolate salts (e.g. ammonium and quaternary alkyl ammonium); chitosan; aloe-vera extracts; algae extract; honey and derivatives thereof; inositol; lactic acid and lactate salts (e.g. ammonium and quaternary alkyl ammonium); sugars and starches (e.g., maltose, glucose, fructose); sugar and starch derivatives (e.g., glucose alkoxylated glucose, mannitol, xyliyol); DL-panthenol; magnesium ascorbyl phosphate, arbutin, kojic acid, lactamide monoethanolamine; acetamide monoethanolamine; and the like, and mixtures thereof. Humectants also include the C₃ to C₆ diols and triols, such as glycerin, propylene glycol, butane-1,2,3-triol, hexylene glycol, hexanetriol, and the like, and mixtures thereof. Ethoxylated methyl glucose ethers containing an average of 5 to 30 moles of ethoxylation, such as, for example, those available under the INCI names Lauryl Methyl Gluceth-10 Hydroxypropyldimonium chloride, Methyl Gluceth-10 and Methyl Gluceth-20, are suitable.

Such humectants may be present at from 0.01-20 wt. % of the composition, such as at least 0.1 wt. %, or at least 1 wt. %, e.g., up to 8 wt. %, or up to 5 wt. %.

Sensates

A skin sensate helps provide a sensory confirmation of the adequacy, activity and evenness of the application thereof by a user. Some non-limiting examples of skin sensates are described in U.S. Pat. Nos. 4,230,688, 4,136,163, 6,183,766 and 7,001,594 each of which are incorporated herein by reference in their entireties. Non-limiting examples of suitable sensates include butanedioic acid monomenthyl ester, camphor, carvone, cineole, clove oil, ethyl carboxamide, ethyl menthane carboxamide, eucalyptus oil, eucolytol, ginger oil, I-isopulegol, menthol, menthone glycerin acetal, menthoxy-1,2-propanediol, menthyl lactate, methyl diisopropylpropioniamide, methyl salicylate, peppermint oil, rosemary oil, trimethyl butanamide, vanillyl butyl ether or combinations thereof. The sensate can be included in the composition in amounts ranging from about 0.01 wt. % to about 2 wt. % in one aspect, and from about 0.05 wt. % to about 1 wt. % in another aspect, based on the total weight of the composition.

Botanicals

The hair care compositions of the disclosed technology can contain one or more botanical agents. Suitable botanical agents can include, for example, extracts from Echinacea (e.g., sp. angustifolia, purpurea, pallida), yucca glauca, willow herb, basil leaves, Turkish oregano, carrot root, grapefruit, fennel seed, rosemary, tumeric, thyme, blueberry, bell pepper, blackberry, spirulina, black currant fruit, tea leaves, such as for, example, Chinese tea, black tea (e.g., var. Flowery Orange Pekoe, Golden Flowery Orange Pekoe, Fine Tippy Golden Flowery Orange Pekoe), green tea (e.g., var. Japanese, Green Darjeeling), oolong tea, coffee seed, dandelion root, date palm fruit, gingko leaf, green tea, hawthorn berry, licorice, apricot kernel, sage, strawberry, sweet pea, tomato, sunflower seed extract, sandalwood extract, grape seed, aloe leaf, vanilla fruit, comfrey, arnica, Centella asiatica, cornflower, horse chestnut, ivy, Macadamia ternifolia seed, magnolia, oat, pansy, skullcap, seabuckthorn, white nettle, and witch hazel. Botanical extracts may also include, for example, chlorogenic acid, glutathione, glycrrhizin, neohesperidin, quercetin, rutin, morin, myricetin, absinthe, and chamomile.

In one aspect, the hair care composition can contain from about 0.01 wt. % to about 10 wt. % of one or more of the botanical extracts set forth above, from about 0.05 wt. % to about to about 5 wt. % in another aspect, from about 0.1 wt. % to about 3 wt. % in still another aspect, and from about 0.5 wt. % to about 1 wt. % in a further aspect, based on the total weight of the composition.

Amino Acids

The hair care composition provided herein can contain one or more non-guanidine moiety containing amino acids. Examples of amino acids that can be used include, without limitation, capryl keratin amino acids, capryl silk amino acids, jojoba amino acids, keratin amino acids, palmitoyl keratin amino acids, palmitoyl silk amino acids, sodium cocoyl amino acids, sodium cocoyl silk amino acids, and sweet almond amino acids.

The hair straightening composition can include an appropriate amount of amino acid(s). The amount of amino acid ranges from about 0.001 wt. % to about 5 wt. % in one aspect, from about 0.01 wt. % percent to about 3 wt. % in another aspect, from about 0.1 wt. % to about 2 wt. % in still another aspect, and from about 0.5 wt. % to about 1 wt. % in a further aspect, based on the total weight of the composition.

Vitamins

The hair care composition can contain one or more vitamins. Examples of vitamins that can be used include, without limitation, niacinamide, sodium starch octenylsuccinate, calcium pantothenate, maltodextrin, sodium ascorbyl phosphate, tocopheryl acetate, pyridoxine HCl, silica, panthenol (e.g., Pro Vitamin B5), phytantriol, calcium pantothenate (e.g., vitamin B5), vitamin E, and vitamin E esters (e.g., tocopheryl acetate, tocopheryl nocotinate, tocopheryl palmitate, or tocopheryl retinoate).

A hair care composition provided herein can include any amount of vitamin(s). The amount of vitamin(s) can range from about 0.05 wt. % to about 10 wt. % in one aspect, from about 0.1 wt. % to about 5 wt. % in another aspect, from about 0.5 wt. % to about 3 wt. % in still another aspect, and from about 0.75 wt. % to about 1 wt. % in a further aspect, based on the total weight of the composition.

Chelating Agents

Chelating agents can be employed to stabilize the composition against the deleterious effects of metal ions. When utilized, suitable chelating agents include EDTA (ethylene diamine tetraacetic acid) and salts thereof such as disodium and tetrasodium EDTA, citric acid and salts thereof, cyclodextrins, and the like, and mixtures thereof.

Such suitable chelating agents can comprise 0.001 wt. % to 3 wt. %, such as 0.01 wt. % to 2 wt. %, or 0.01 wt. % to 1 wt. % of the total weight of the hair straightening composition.

Buffer Agents

Buffering agents can be used in the exemplary compositions. Suitable buffering agents include alkali or alkali earth metal carbonates, phosphates, bicarbonates, citrates, borates, acetates, acid anhydrides, succinates, and the like, such as sodium phosphate, sodium citrate, sodium acetate, sodium bicarbonate, and sodium carbonate.

pH Adjusting Agents

The pH of the composition can range from to 1.5 to 9.5 in one aspect, at least 4.5 in a second aspect, at least 5.5 a third aspect, at least 6.5 in a fourth aspect, at least 7.0 in a fifth aspect, at least 7.5 in a sixth aspect, at least 8.0 in a seventh aspect, at least 8.5 in an eighth aspect, at least 9.0 in a ninth aspect, and at least 9.5 in a tenth aspect.

When polyvalent metal salts of pyrithione in combination with secondary zinc salts are employed in the antidandruff hair care compositions of the disclosed technology, the pH of the composition is adjusted to a value of at least about 6.5. The pH can range from about 6.5 to about 12 in one aspect, from about 6.8 to about 9.5 in another aspect, and from about 6.8 to about 8.5 in still another aspect. To provide the desired pH, the composition may be adjusted with one or more pH modifiers selected from organic and inorganic acids and bases.

The pH of the composition can be adjusted with any combination of acidic and/or basic pH adjusting agents known to the art. Acidic materials include organic acids and inorganic acids, in particular, monocarboxylic acids, dicarboxylic acids, and tricarboxylic acids, for example, acetic acid, citric acid, tartaric acid, alpha-hydroxy acids, beta-hydroxy acids, salicylic acid, lactic acid, malic acid, glycolic acid, amino acids, and natural fruit acids, or inorganic acids, for example, hydrochloric acid, nitric acid, sulfuric acid, sulfamic acid, phosphoric acid, and combinations thereof.

Basic materials include inorganic and organic bases, and combinations thereof. Examples of inorganic bases include but are not limited to the alkali metal hydroxides (e.g., potassium hydroxide, sodium hydroxide) and alkali metal carbonates (e.g., potassium carbonate, sodium carbonate), and alkali metal salts such as sodium borate (borax), sodium phosphate, sodium pyrophosphate, and the like; and mixtures thereof. Examples of organic bases include ammonium hydroxide, triethanolamine (TEA), diisopropanolamine, triisopropanolamine, aminomethyl propanol, dodecylamine, cocamine, oleamine, morpholine, triamylamine, triethylamine, tetrakis(hydroxypropyl)ethylenediamine, L-arginine, aminomethyl propanol, tromethamine (2-amino 2-hydroxymethyl-1,3-propanediol), and PEG-15 cocamine.

The pH adjusting agent(s) and/or buffering agent is utilized in any amount necessary to obtain and/or maintain a desired pH value in the composition.

Preservatives

In one aspect, any preservative suitable for use in personal care can be used in the composition for straightening hair. Suitable preservatives include polymethoxy bicyclic oxazolidine, methyl paraben, propyl paraben, ethyl paraben, butyl paraben, benzyltriazole, DMDM hydantoin (also known as 1,3-dimethyl-5,5-dimethyl hydantoin), imidazolidinyl urea, phenoxyethanol, phenoxyethylparaben, methylisothiazolinone, methylchloroisothiazolinone, benzoisothiazolinone, triclosan, and suitable polyquaternium compounds as disclosed above (e.g., Polyquaternium-1).

In another aspect, acid based preservatives are useful in the exemplary compositions. The use of acid based preservatives facilitates the formulation of products in the low pH range. Lowering the pH of a formulation inherently provides an inhospitable environment for microbial growth in addition to being suited to the straightening process. Moreover, formulating at low pH enhances the efficacy of acid based preservatives, and affords a personal care product which maintains an acidic pH balance on the skin. Any acid based preservative that is useful in personal care products can be used in the exemplary compositions. In one aspect the acid preservative is a carboxylic acid compound represented by the formula: R⁸⁰C(O)OH, wherein R⁸⁰ represents hydrogen, a saturated and unsaturated hydrocarbyl group containing 1 to 8 carbon atoms or C₆ to C₁₀ aryl. In another aspect, R⁸⁰ is selected from a hydrogen, a C₁ to C₈ alkyl group, a C₂ to C₈ alkenyl group, or phenyl. Exemplary acids are, but are not limited to, formic acid, acetic acid, propionic acid, sorbic acid, caprylic acid, and benzoic acid, and mixtures thereof.

In another aspect, suitable acids include but are not limited to, oxalic acid, succinic acid, glutaric acid, adipic acid, azelaic acid, maleic acid, fumaric acid, lactic acid, glyceric acid, tartronic acid malic acid, tartaric acid, gluconic acid, citric acid, ascorbic acid, salicylic acid, phthalic acid, mandelic acid, benzilic acid, and mixtures thereof.

Salts of the foregoing acids are also useful as long as they retain efficacy at low pH values. Suitable salts include the alkali metal (e.g., sodium, potassium, calcium) and ammonium salts of the acids enumerated above.

The acid based preservatives and/or their salts can be used alone or in combination with non-acidic preservatives typically employed in personal care, home care, health care, and institutional and industrial care products.

The preservatives may comprise from 0.01 wt. % to 3.0 wt. % in one aspect, or from about 0.1 wt. % to about 1 wt. %, or from about 0.3 wt. % to about 1 wt. %, of the total weight of the hair care composition.

Perfumes and Fragrances

Fragrance and perfume components that may be used in the exemplary composition to mask the odor of any of the various components in the hair straightening composition or to give the composition an aesthetically pleasing fragrance. In one aspect, suitable fragrances and perfumes include natural and synthetic fragrances, perfumes, scents, and essences and any other substances which emit a fragrance. As the natural fragrances, there are those of vegetable origin, such as oil extracts from flowers (e.g., lily, lavender, rose, jasmine, neroli, ylang-ylang), stems and leaves (geranium, patchouli, petitgrain, peppermint), fruits (aniseed, coriander, fennel, mace, needle juniper), fruit skin (bergamot, lemon, orange), roots (angelica, celery, cardamom, costus, iris, sweet flag), woods (pine tree, sandalwood, guaiacum wood, cedar, rosewood, cinnamon), herbs and grasses (tarragon, lemongrass, sage, thyme), needles and twigs (spruce, pine, European red pine, stone pine), and resins and balsam (galbanum, elemi, benzoin, myrrh, frankincense, opopanax), and those of animal origin, such as musk, civet, castoreum, ambergris, or the like, and mixtures thereof.

Examples of synthetic fragrances and perfumes are the aromatic esters, ethers, aldehydes, ketones, alcohols, and hydrocarbons including benzyl acetate, phenoxyethyl isobutylate, p-tert-butylcyclohexyl acetate, linalyl acetate, dimethylbenzylcarbinyl acetate, phenylethyl acetate, linalyl benzoate, benzyl formate, ethylmethylphenyl glycinate, allylcyclohexyl propionate, styralyl propionate, and benzyl salicylate; benzylethyl ether; straight chain alkanals having 8 to 18 carbon atoms, citral, citronellal, citronellyloxyaldehyde, cyclamen aldehyde, hydroxycitronellal, lilial, and bougeonal; ionone compounds, α-isomethyl ionone, and methyl cedryl ketone; anethole, citronellol, eugenol, isoeugenol, geraniol, lavandulol, nerolidol, linalool, phenylethyl alcohol, and terpineol, alpha-pinene, terpenes (e.g., limonene), and balsams, and mixtures thereof.

The amount of fragrance agent or perfume employed can be any amount suitable to mask a particular odor or to impart a desired aesthetically pleasing aroma, fragrance or scent. In one aspect, the amount of fragrance agent can range from about 0.05 wt. % to about 10 wt. %, from about 0.1 wt. % to about 5 wt. % in another aspect, from about 0.5 wt. % to about 3.5 wt. % in still another aspect, and from about 1 wt. % to about 2.5 wt. % in a further aspect, based on the total weight of the composition.

Electrolytes

Optionally, the cleansing and conditioning compositions of the disclosed technology can contain an electrolyte. Suitable electrolytes are known compounds and include salts of multivalent anions, such as potassium pyrophosphate, potassium tripolyphosphate, and sodium or potassium citrate, salts of multivalent cations, including alkaline earth metal salts such as calcium chloride and calcium bromide, as well as zinc halides, barium chloride, magnesium sulfate and calcium nitrate, salts of monovalent cations with monovalent anions, including alkali metal or ammonium halides, such as potassium chloride, sodium chloride, potassium iodide, sodium bromide, and ammonium bromide, alkali metal or ammonium nitrates, and blends thereof. The amount of the electrolyte used will generally depend on the amount of the amphiphilic emulsion polymer incorporated, but may be used at concentration levels of from about 0.1 to about 4 wt. % in one aspect and from about 0.2 to about 2 wt. % in another aspect, based on the weight of the total composition.

Dyes and Pigments

The hair care compositions of the present technology may also contain pigment materials such as inorganic, nitroso, monoazo, disazo, carotenoid, triphenyl methane, triaryl methane, xanthene, quinoline, oxazine, azine, anthraquinone, indigoid, thionindigoid, quinacridone, phthalocianine, botanical, natural colors, including: water soluble components such as those having C. I. and FD&C designations.

Exemplary pigments are metal compounds or semi metallic compounds and may be used in ionic, nonionic or oxidized form. The pigments can be in this form either individually or in admixture or as individual mixed oxides or mixtures thereof, including mixtures of mixed oxides and pure oxides. Examples are the titanium oxides (e.g., TiO2), zinc oxides (e.g., ZnO), aluminum oxides (for example, Al₂O₃), iron oxides (for example, Fe₂O₃), manganese oxides (e.g., MnO), silicon oxides (e.g., SiO2), silicates, cerium oxides, zirconium oxides (e.g., ZrO2), barium sulfate (BaSO4), nylon-12, and mixtures thereof.

Other examples of pigments include thermochromic dyes that change color with temperature, calcium carbonate, aluminum hydroxide, calcium sulfate, kaolin, ferric ammonium ferrocyanide, magnesium carbonate, carmine, barium sulfate, mica, bismuth oxychloride, zinc stearate, manganese violet, chromium oxide, titanium dioxide nanoparticles, barium oxide, ultramarine blue, bismuth citrate, hydroxyapatite, zirconium silicate, carbon black particles, and the like.

Fixatives

Suitable hair fixative polymers include natural and synthetic polymers such as, for example, polyacrylates, polyvinyls, polyesters, polyurethanes, polyamides, modified cellulose, starches, and mixtures thereof. These polymers can be nonionic, anionic, cationic and amphoteric in nature and include without limitation one or more of polyoxyethylenated vinyl acetate/crotonic acid copolymers, vinyl acetate crotonic acid copolymers, vinyl methacrylate copolymers, monoalkyl esters of poly(methyl vinyl ether (PVM)/maleic anhydride (MA)), such as, for example, ethyl, butyl and isopropyl esters of PVM/MA copolymer acrylic acid/ethyl acrylate/N-tert-butyl-acrylamide terpolymers, and poly (methacrylic acid/acrylamidomethyl propane sulfonic acid), acrylates copolymer, octylacrylamide/acrylates/butylaminoethyl methacrylate copolymer, acrylates/octylacrylamide copolymer, vinyl acetate (VA)/crotonates/vinyl neodeanoate copolymer, poly(N-vinyl acetamide), poly(N-vinyl formamide), corn starch modified, sodium polystyrene sulfonate, polyquaterniums such as, for example, Polyquaternium-4, Polyquaternium-5, Polyquaternium-6, Polyquaternium-7, Polyquaternium-10, Polyquaternium-11, Polyquaternium-22, Polyquaternium-24, Polyquaternium-28, Polyquaternium-29, Polyquaternium-32, Polyquaternium-34, Polyquaternium-37, Polyquaternium-39, Polyquaternium-44, Polyquaternium-46, Polyquaternium-47, Polyquaternium-52, Polyquaternium-53, Polyquarternium-55, Polyquaternium-68, Polyquaternium-69, Polyquaternium-87, Laureth-16, polyether-1, VA/acrylates/lauryl methacrylate copolymer, adipic acid/dimethylaminohydroxypropyl diethylene AMP/acrylates copolymer, methacrylol ethyl betaine/acrylates copolymer, acrylamide/sodium acryloyldimethyltaurate/acrylic acid, polyvinylpyrrolidone (PVP), vinyl pyrrolidone (VP)/dimethylaminoethylmethacrylate copolymer, acrylic acid/VP crosspolymer, VP/methacrylamide/vinyl imidazole copolymer, VP/dimethylaminopropylamine (DMAPA) acrylates copolymer, VP/vinylcaprolactam/DMAPA acrylates copolymer, vinyl caprolactam/VP/dimethylam inoethyl methacrylate copolymer, VA/butyl maleate/isobornyl acrylate copolymer, VA/crotonates copolymer, acrylate/acrylamide copolymer, VA/crotonates/vinyl propionate copolymer, VP/vinyl acetate/vinyl propionate terpolymers, VP/vinyl acetate copolymer, VP/acrylates copolymer, VA/crotonic acid/vinyl proprionate, acrylates/acrylamide, acrylates/octylacrylamide, acrylates/hydroxyacrylates copolymer, acrylates/hydroxyesteracrylates copolymer, acrylates/stereth-20 methacrylate copolymer, tert-butyl acrylate/acrylic acid copolymer, diglycol/cyclohexanedimethanol/isophthalates/sulfoisophthalates copolymer, VA/alkylmaleate half ester/N-substituted acrylamide terpolymers, vinyl caprolactam/VP/methacryloamidopropyl trimethylammonium chloride terpolymer, methacrylates/acrylates copolymer/amine salt, polyvinylcaprolactam, hydroxypropyl guar, poly (methacrylic acid/acrylamidomethyl propane sulfonic acid (AMPSA), ethylenecarboxamide (EC)/AMPSA/methacrylic acid (MAA), poylurethane/acrylate copolymers and hydroxypropyl trimmonium chloride guar, acrylates crosspolymer, acrylates crosspolymer-3, AMP-acrylates/allyl methacrylate copolymer, polyacrylate-14, polyacrylate-2 crosspolymer, acrylates/lauryl acrylate/stearyl acrylate/ethylamine oxide methacrylate copolymer, methacryloyl ethyl betaines/methacrylates copolymer, polyurethane/acrylates copolymer, pyrrolidone carboxylic acid salt of chitosan, chitosan glycolate, cationic polygalactomannans, such as, for example, quaternized derivatives of guar, such as, for example, guar hydroxypropyl trimmonium chloride, cassia hydroxypropyl trimmonium chloride and hydroxypropyl guar hydroxypropyl trimmonium chloride. Many of the foregoing polymers are referred to by their INCI nomenclature set forth in the International Cosmetic Ingredient Dictionary published by the Cosmetic, Toiletry, and Fragrance Association, Washington D.C. Other suitable auxiliary fixative polymers are disclosed in U.S. Pat. No. 7,205,271, the disclosure of which is herein incorporated by reference.

The fixative polymer typically comprises about 0.01 wt. % to about 8 wt. % in one aspect, from about 0.1 wt. % to about 5 wt. % in another aspect, and about 0.2 wt. % to about 3 wt. % in a further aspect of the total weight of the hair styling composition.

Detersive Compositions

Body Wash

In one aspect, a personal care composition in which the polymer of this technology is useful is a body wash. Typical components of a body wash, in addition to the polymer thickener and water are: at least one surfactant; a sufficient pH adjusting agent (base and/or acid) to attain a pH of from about 3.5 to about 7.5 in one aspect, from about 4.0 to about 6.5 in another aspect, and from about 5.0 to about 6.0 in a further aspect; and optional ingredients selected from the adjuvants, additives and benefit agents discussed above, and mixtures thereof, including benefit agents selected from silicones, pearlizing agents, vitamins, oils, fragrances, dyes, preservatives including acids, botanicals, exfoliating agents, insoluble gas bubbles, liposomes, microsponges, cosmetic beads and flakes. In one aspect, the surfactant is an anionic surfactant. In another aspect, the surfactant is a mixture of an anionic surfactant and an amphoteric surfactant, in optional combination with a non-ionic surfactant. In another aspect, the surfactant is a mixture of an anionic surfactant and an amphoteric surfactant, in optional combination with a cationic and/or a non-ionic surfactant. In one aspect, the anionic surfactant can be present in an amount ranging from about 5% to about 40% by weight, from about 6% to about 30% by weight in another aspect, and from 8% to about 25% by weight in a further aspect, based on the total weight of the body wash composition. When mixtures of anionic and amphoteric surfactants are used, the ratio of anionic surfactant:amphoteric surfactant can range from about 1:1 to about 15:1 in one aspect, from about 1.5:1 to about 10:1 in another aspect, from about 2.25:1 to about 9:1 in a further aspect, and from about 4.5:1 to about 7:1 in a still further aspect. The amount of the acrylic polymer blend(s) can range from about 0.5% to about 5% by weight in one aspect, from about 1% to about 3% by weight in another aspect, and from about 1.5% to about 2.5% by weight in a further aspect, based on the total weight of the body wash composition.

Body wash embodiments of the technology can be formulated as moisturizing body washes, antibacterial body washes, dual purpose body wash and shampoo, bath gels, shower gels, liquid hand soaps, body scrubs; bubble baths, facial scrubs, foot scrubs, and the like.

Shampoo

In one aspect, a personal care composition in which the polymer of this technology is useful is a shampoo. Typical components of a shampoo, in addition to the polymer thickener and water are: at least one surfactant; a sufficient pH adjusting agent (base and/or acid) to attain a pH of from about 3.0 to about 7.5 in one aspect, from about 3.5 to about 6.0 in another aspect, and from about 4.0 to about 5.5 in a further aspect; and optional ingredients selected from the adjuvants, additives and benefit agents discussed above, and mixtures thereof, including benefit agents selected from conditioning agents (e.g., silicones and/or cationic conditioning agents; small and/or large particle sized silicones), pearlizing agents, vitamins, oils, fragrances, dyes, preservatives including acids, botanicals, and insoluble gas bubbles, liposomes, and cosmetic beads and flakes, and anti-dandruff agents, and mixtures thereof. In one aspect, the surfactant is an anionic surfactant. In another aspect, the surfactant is a mixture of an anionic surfactant and an amphoteric surfactant, in optional combination with a cationic and/or a non-ionic surfactant. In one aspect, the anionic surfactant can be present in an amount ranging from about 5% to about 40% by weight, from about 6% to about 30% by weight in another aspect, and from 8% to about 25% by weight in a further aspect, based on the total weight of the shampoo composition. When mixtures of anionic and amphoteric surfactants are used, the ratio of anionic surfactant to amphoteric surfactant can range from about 1:1 to about 10:1 in one aspect, from about 2.25:1 to about 9:1 in another aspect, and from about 4.5:1 to about 7:1 in a further aspect. The amount of I polymer can range from about 0.5% to about 5% by weight in one aspect, from about 1% to about 3% by weight in another aspect, and from about 1.5% to about 2.5% by weight in a further aspect, based on the total weight of the shampoo composition.

The shampoo embodiments of the disclosed technology can be formulated as 2-in-1 shampoos, baby shampoos, conditioning shampoos, bodifying shampoos, moisturizing shampoos, temporary hair color shampoos, 3-in-1 shampoos, anti-dandruff shampoos, hair color maintenance shampoos, acid (neutralizing) shampoos, medicated shampoos, and salicylic acid shampoos, and the like.

Fatty Acid Liquid Soap Based Cleansers

In one aspect, a personal care composition in which the polymer of this technology is useful is a fatty acid soap based cleanser. Typical components of a fatty acid based soap cleanser, in addition to the polymer thickener are: at least one fatty acid salt; an optional surfactant or mixture of surfactants; a sufficient pH adjusting agent (base and/or acid) to attain a pH of above 7 in one aspect, from about 7.5 to about 14 in another aspect, from about 8 to about 12 in still another aspect, and from about 8.5 to about 10 in a further aspect; and optional ingredients selected from the adjuvants, additives and benefit agents discussed above, and mixtures thereof, including benefit agents selected from silicones, humectants, pearlizing agents, vitamins, oils, fragrances, dyes, preservatives, botanicals, anti-dandruff agents, exfoliating agents, insoluble gas bubbles, liposomes, microsponges, cosmetic beads and flakes.

In one aspect, the fatty acid soaps are selected from at least one the fatty acid salt (e.g., sodium, potassium, ammonium) containing from about 8 to about 22 carbon atoms. In another aspect of the technology the liquid soap composition contains at least one fatty acid salt containing from about 12 to about 18 carbon atoms. The fatty acids utilized in the soaps can be saturated and unsaturated and can be derived from synthetic sources, as well as from the saponification of fats and natural oils by a suitable base (e.g., sodium, potassium and ammonium hydroxides). Exemplary saturated fatty acids include but are not limited to octanoic, decanoic, lauric, myristic, pentadecanoic, palmitic, margaric, steric, isostearic, nonadecanoic, arachidic, behenic, and the like, and mixtures thereof. Exemplary unsaturated fatty acids include but are not limited to the salts (e.g., sodium, potassium, ammonium) of myristoleic, palmitoleic, oleic, linoleic, linolenic, and the like, and mixtures thereof. The fatty acids can be derived from animal fat such as tallow or from vegetable oil such as coconut oil, red oil, palm kernel oil, palm oil, cottonseed oil, olive oil, soybean oil, peanut oil, corn oil, and mixtures thereof. The amount of fatty acid soap that can be employed in the liquid cleansing compositions of this embodiment ranges from about 1% to about 50% by weight in one aspect, from about 10% to about 35% by weight in another aspect, and from about 12% to 25% by weight in a further aspect of the technology, based on the weight of the total composition.

An optional anionic surfactant can be present in the soap composition in an amount ranging from about 1% to about 25% by weight in one aspect, from about 5% to about 20% by weight in another aspect, and from 8% to about 15% by weight in a further aspect, based on the weight of the total weight of the soap composition. Mixtures of anionic and amphoteric surfactants can be used. The ratio of anionic surfactant to amphoteric surfactant can range from about 1:1 to about 10:1 in one aspect, from about 2.25:1 to about 9:1 in another aspect, and from about 4.5:1 to about 7:1 in a further aspect.

In the foregoing soap embodiments of the technology, the amount of polymer can range from about 0.5% to about 5% by weight in one aspect, from about 1% to about 3% by weight in another aspect, and from about 1.5% to about 2.5% by weight in a further aspect, based on the total weight of the soap composition.

The liquid fatty acid soap based cleanser embodiments of the technology can be formulated as body washes, bath gels, shower gels, liquid hand soaps, body scrubs; bubble baths, facial scrubs, and foot scrubs, 2-in-1 shampoos, baby shampoos, conditioning shampoos, bodifying shampoos, moisturizing shampoos, temporary hair color shampoos, 3-in-1 shampoos, anti-dandruff shampoos, hair color maintenance shampoos, acid (neutralizing) shampoos, anti-dandruff shampoos, medicated shampoos, and salicylic acid shampoos, and the like.

Cosmeceuticals

In one cosmeceutical aspect, the polymers of the technology can be employed as a thickener for active skin treatment lotions and creams containing, as active ingredients anti-aging, anti-cellulite and anti-acne agents. Exemplary active ingredients include alpha-hydroxy acid (AHA), beta-hydroxy acid (BHA), alpha-amino acid, alpha-keto acids (AKAs), and mixtures thereof. In one aspect, AHAs can include, but are not limited to, lactic acid, glycolic acid, fruit acids, such as malic acid, citric acid, tartaric acid, extracts of natural compounds containing AHA, such as apple extract, apricot extract, and the like, honey extract, 2-hydroxyoctanoic acid, glyceric acid (dihydroxypropionic acid), tartronic acid (hydroxypropanedioic acid), gluconic acid, mandelic acid, benzilic acid, azelaic acid, alpha-lipoic acid, salicylic acid, AHA salts and derivatives, such as arginine glycolate, ammonium glycolate, sodium glycolate, arginine lactate, ammonium lactate, sodium lactate, alpha-hydroxybutyric acid, alpha-hydroxyisobutyric acid, alpha-hydroxyisocaproic acid, alpha-hydroxyisovaleric acid, atrolactic acid, and the like. BHAs can include, but are not limited to, 3-hydroxy propanoic acid, beta-hydroxybutyric acid, beta-phenyl lactic acid, beta-phenylpyruvic acid, and the like. Alpha-amino acids include, without being limited thereto, alpha-amino dicarboxylic acids, such as aspartic acid, glutamic acid, and mixtures thereof, sometimes employed in combination with fruit acid. AKAs include pyruvic acid. In some antiaging compositions, the acidic active agent may be retinoic acid, a halocarboxylic acid, such as trichloroacetic acid, an acidic antioxidant, such as ascorbic acid (vitamin C), a mineral acid, phytic acid, lysophosphatidic acid, and the like. Some acidic anti-acne actives, for example, can include salicylic acid, derivatives of salicylic acid, such as 5-octanoylsalicylic acid, retinoic acid, and its derivatives, and benzoic acid.

A discussion of the use and formulation of active skin treatment compositions is in COSMETICS & TOILETRIES, C&T Ingredient Resource Series, “AHAs & Cellulite Products How They Work”, published 1995, and “Cosmeceuticals”, published 1998, both available from Allured Publishing Corporation, incorporated herein by reference. Compositions containing alpha-amino acids acidified with ascorbic acid are described in U.S. Pat. No. 6,197,317 B1, and a commercial cosmeceutical preparation utilizing these acids in an anti-aging, skin care regimen is sold under the tradename, AFAs, by exCel Cosmeceuticals (Bloomfield Hills, Mich.). The term “AFA”, as described in the supplier's trade literature, was coined by the developer to describe the amino acid/vitamin C combination as Amino Fruit Acids and as the acronym for “Amino acid Filaggrin based Antioxidants.”

Health Care

Health care embodiments in which the instant polymers can be included are medical products, such as topical and non-topical pharmaceuticals, and devices. In the formulation of pharmaceuticals, a polymer embodiment of the technology can be employed as a thickener and/or lubricant in such products as syrups, creams, pomades, gels, pastes, ointments, tablets, gel capsules, purgative fluids (enemas, emetics, colonics, and the like), suppositories, anti-fungal foams, eye products (ophthalmic products, such as eye drops, artificial tears, glaucoma drug delivery drops, contact lens cleaner, and the like), ear products (wax softeners, wax removers, otitis drug delivery drops, and the like), nasal products (drops, ointments, sprays, and the like), and wound care (liquid bandages, wound dressings, antibiotic creams, ointments, and the like), without limitation thereto.

Other health care embodiments relate to foot care products, such as keratolytic corn and callous removers, foot soaks, medicated foot products such as antifungal athlete's foot ointments, gels, sprays, and the like, as well as antifungal, anti-yeast, and antibacterial creams, gels, sprays, and ointments.

Other health care embodiments relate to foot care products, such as keratolytic corn and callous removers, foot soaks, medicated foot products such as antifungal athlete's foot ointments, gels, sprays, and the like, as well as antifungal, anti-yeast, and antibacterial creams, gels, sprays, and ointments.

Hair care compositions of the present technology are stable indefinitely at temperatures normally found in commercial product storage and shipping. The compositions resist phase separation or settling of composition ingredients at a temperature of about 20° C. to about 25° C. essentially indefinitely. The compositions also must demonstrate sufficient stability to phase separation and settling of ingredients at temperatures normally found in commercial product storage and shipping to remain unaffected for periods of one year or more.

The cleansing compositions employing the HASE polymers of the disclosed technology not only provide compositions in which they are contained with enhanced suspension stability, they also provide other unexpected desirable properties such as foam quality, and irritation mitigation.

This technology is illustrated by the following examples that are merely for the purpose of illustration and are not to be regarded as limiting the scope of the technology or the manner in which it can be practiced. Unless specifically indicated otherwise, parts and percentages are given by weight.

Test Methods

Yield Stress

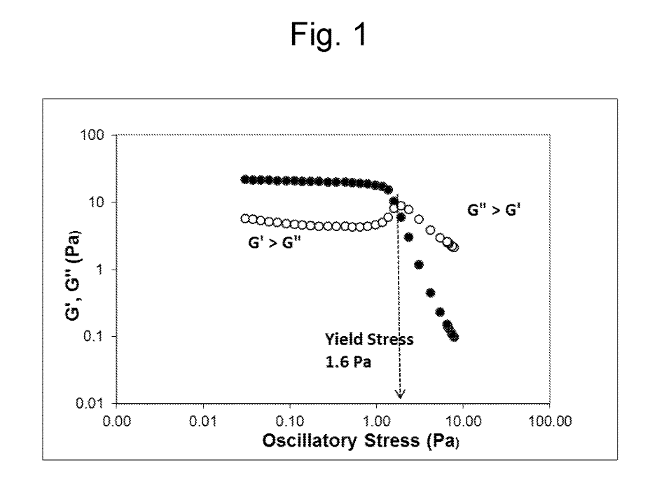

The yield stress values of these polymers are determined by oscillatory and steady shear measurements on a controlled stress rheometer (TA Instruments AR1000N rheometer, New Castle, Del.) utilizing 40 mm or 60 mm stainless steel cone-plate 2 degree geometry at 25° C. The oscillatory measurements are performed at a fixed frequency of 1 rad/sec. The elastic and viscous moduli (G′ and G″ respectively) are obtained as a function of increasing stress amplitude. In cases where the swollen polymer particles create a network, G′ is larger than G″ at low stress amplitudes but decreases at higher amplitudes crossing G″ because of rupture of the network. The oscillatory stress corresponding to the crossover of G′ and G″ is noted as the yield stress (FIG. 1 illustrates a representative yield stress plot).

Viscosity (Brookfield)

Brookfield rotating spindle method (unless otherwise specified all viscosity measurements reported herein are conducted by the Brookfield method): The viscosity measurements are calculated in mPa·s, employing a Brookfield rotating spindle viscometer, Model RVT (Brookfield Engineering Laboratories, Inc.), at about 20 revolutions per minute (rpm), at ambient room temperature of about 20 to 25° C. (hereafter referred to as viscosity). Spindle sizes are selected in accordance with the standard operating recommendations from the manufacturer. Generally, spindle sizes are selected as follows:

| Spindle Size No. | Viscosity Range (mPa · s) |
|---|---|
| 1 | 1-50 |
| 2 | 500-1,000 |
| 3 | 1,000-5,000 |
| 4 | 5,000-10,000 |
| 5 | 10,000-20,000 |
| 6 | 20,000-50,000 |
| 7 | >50,000 |

The spindle size recommendations are for illustrative purposes only. The artisan of ordinary skill in the art will select a spindle size appropriate for the system to be measured. Unless specified otherwise viscosity was measured 8 hrs. after the sample was formulated.

Viscosity (AR-G2 Rheometer)

Samples evaluated for viscosity properties on the AR-G2 rheometer, TA Instruments, were subjected to a shear rate of 3.5 s⁻¹ at temperature of 25° C., for one minute using standard 40 mm steel parallel plate geometry with a gap of 1000 μm. Prior to each of the measurements, the sample compositions were loaded and idled for 5 minutes, to allow the sample compositions to reach equilibrium.

Clarity

The clarity (turbidity) of a composition is determined in Nephelometric Turbidity Units (NTU) employing a nephelometric turbidity meter (Mircro 100 Turbidimeter, HF Scientific, Inc.) at ambient room temperature of about 20 to 25° C. Distilled water (NTU=0) is utilized as a standard. Six dram screw cap vials (70 mm×25 mm) are filled almost to the top with test sample and centrifuged at 100 rpm until all bubbles are removed. Upon centrifugation, each sample vial is wiped with tissue paper to remove any smudges before placement in the turbidity meter. The sample is placed in the turbidity meter and a reading is taken. Once the reading stabilizes the NTU value is recorded. The vial is given one-quarter turn and another reading is taken and recorded. This is repeated until four readings are taken. The lowest of the four readings is reported as the turbidity value.

Transmittance

When reported, the transmittance of the polymer-containing composition is measured in percent T (transmittance) by Brinkmann PC 920 colorimeter at least about 24 hours after the composition is made. Clarity measurements are taken against deionized water (clarity rating of 100 percent). Compositions having a clarity of about 60 percent or more are substantially clear; compositions having a clarity in the range of about 45 percent to 59 percent are judged substantially translucent.

Suspension Stability

The various compositions made using the HASE rheology polymers of the present technology are stable. The stability requirements for a particular composition will vary with its end marketplace application as well as the geography in which it is to be bought and sold. An acceptable “shelf life” is subsequently determined for each composition. This refers to the amount of time that a composition should be stable across its normal storage and handling conditions, measured between the times that the composition is produced and when it is finally sold for consumer use. Generally, Personal Care compositions require a 1 to 3 year shelf life.

To eliminate the need to conduct stability studies in excess of one year, the formulator will conduct stability testing at stressed conditions in order to predict the shelf life of a composition. Typically, accelerated testing is conducted at elevated static temperatures, usually 45-50° C. A composition should be stable for at least 1 week in one aspect, at least 1 month in another aspect, at least about 3 months in a still another aspect, and at least about 6 months in a further aspect at a temperature of about 45° C.

The ability of a polymer system to suspend active and/or aesthetically pleasing insoluble oily and particulate materials is important from the standpoint of product efficacy and appeal. The ability of a test formulation to stably suspend test beads is indicative of its capability to suspend insoluble or particulate materials. A six dram vial (approximately 70 mm high×25 mm in diameter) is filled to the 50 mm point with a test formulation. Each sample vial is centrifuged to remove any trapped air bubbles contained in the formulation. About 10 test beads are placed in each test formulation and stirred gently with a wooden stick until they are uniformly dispersed throughout the sample. The position of approximately 4 of the beads within each sample vial is noted by drawing a circle around the bead with black marker pen on the outer glass surface of the vial and photographed to establish the initial position of the beads within the formulation. The vials are placed in a 45° C. oven to age for a 1 to 12 week period. The bead suspension properties of each sample are visually evaluated at the conclusion of the test period. If the initial position of all 4 of the circled beads is unchanged (3 mm above or below its initial position) following the conclusion of the test period the sample passes. If the initial position of one or more of the 4 circled beads changes (3 mm above or below its initial position) following the conclusion of the test period the sample fails.

Three types of beads are evaluated in the suspension stability test: Type 1. Large sized, hard-to-suspend bead: LipoPearl™ LTI-0293 (Color-White), average particle size around 1,000-2,800 microns, containing Vitamin E, Mineral Oil, Mica, Titanium Dioxide and Gelatin, supplied by Lipo Chemicals, Inc.

Type 2. Medium sized bead: Vision Beads™ GVBGSO/TA, particle size about 1000 microns, containing Grape Seed Oil, Lactose Monohydrate, Microcrystalline Cellulose and Hypromellose, from Impact Colors, Inc. Type 3. Small sized bead: Unispheres® UEA-509, particle size about 500 to 900 microns, containing Vitamin E, Retinyl Palmitate, Lactose, Cellulose and Hydroxypropyl Methylcellulose, supplied by Induchem AG.

Freeze-Thaw Stability

Freeze-thaw stability is tested in 3 freeze-thaw cycles. In each cycle, the sample is frozen at −20° C. for 24 hours, and then thawed at room temperature (between 20-25° C.) for 24 hours. Sample viscosity and clarity are tested after each freeze-thaw cycle. To pass the freeze-thaw stability test, the sample should have no change in appearance, equal or higher clarity (measured as turbidity) and a viscosity change of no more than 25% after 3 freeze-thaw cycles, compared to an identical sample stored at room temperature for 24 hours.

Products or compositions made according to the present technology are considered stable if they meet one or more of the following criteria:

1. There is no phase separation, settling, or creaming of any material in the composition. The composition should remain completely homogenous throughout its bulk. Separation is defined as the visible existence of 2 or more distinct layers or phases of any component in the formulation, including but not limited to insoluble materials, soluble materials, oily substances and the like.

2. The viscosity of the composition does not significantly increase or decrease over time, generally less than 50%, preferably less than 35%, and most preferably less than 20%.

3. The pH of the composition does not increase or decrease more than two pH units, preferably not more than one unit, and most preferably not more than one-half unit.

4. The rheology and texture of the composition does not significantly change over time to that which is unacceptable.

Products or compositions made according to the present technology are considered unstable if they do not meet one or more of the above listed criteria. Further information on stability testing requirements can be found in “The Fundamentals of Stability Testing; IFSCC Monograph Number 2”, published on behalf of the International Federation of Societies of Cosmetic Chemists by Micelle Press, Weymouth, Dorset, England, and Cranford, N.J., U.S.A. and is herein incorporated by reference.

The following abbreviations and trade names are utilized in the examples.

Abbreviations and Trade Names

| | |
|---|---|
| Acrylates Crosspolymer-3 (INCI Name) | Fixate ™ Freestyle Fixative Polymer, |
| | Copolymer Of Acrylic Acid, Methacrylic Acid or |
| | One of Its Simple Esters, Crosslinked with |
| | Trimethylolpropane Triacrylate and |
| | Trimethylolpropane Diallyl Ether, Lubrizol |
| | Advanced Materials, Inc. |
| Acrylates/Steareth-20 Methacrylate | Aculyn ™ 88 HASE Polymer (29% Active), A |
| Crosspolymer (INCI Name) | Copolymer of Steareth-20 Methacrylate and |
| | one or More Monomers Consisting of Acrylic |
| | Acid, Methacrylic Acid or one of Their Simple |
| | Esters, Crosslinked With an Allyl Ether of |
| | Pentaerythritol or an Allyl Ether of |
| | Trimethylolpropane, Dow Chemical. |
| Acrylates/Vinyl Neodecanoate | Aculyn ™ 38 Polymer ASE Polymer (29% |
| Crosspolymer (INCI Name) | Active), A Copolymer of Vinyl Neodecanoate |
| | and one or More Monomers Consisting of |
| | Acrylic Acid, Methacrylic Acid or one of Their |
| | Simple Esters, Crosslinked With an Allyl Ether |
| | of Pentaerythritol or an Allyl Ether of |
| | Trimethylolpropane, Dow Chemical |
| ALES-3 Surfactant | Sulfochem ™ EA-3K Ammonium Laureth |
| | Sulfate, 3 moles of ethoxylation, Lubrizol |
| | Advanced Materials, Inc. (26.1% active) |
| ALS Surfactant | Ammonium Lauryl Sulfate, Sulfochem ™ ALS-K |
| | (29.1% active), Lubrizol Advanced Materials, |
| | Inc. |
| AM | E-Sperse ® RS-1618 Amphiphilic |
| | Macromonomer With Two Ethylenic Reactive |
| | Groups, Ethox Chemical, LLC |
| AMD | Acrylamide |
| Aminomethyl Propanol (INCI) | AMP-Ultra ™ PC 2000 Amino Alcohol, Angus |
| | Chemical Company |
| AOS Surfactant | Sodium C₁₄-C₁₆ alpha olefin sulfonate (≈ 40% |
| | active), Bio-Terge ® AS-40K surfactant, Stepan |
| | Company |
| APE | Allyl Pentaerythritol |
| n-BA | n-Butyl Acrylate |
| BEM | Sipomer ® Ethoxylated (25 moles) Behenyl |
| | Methacrylate, (as supplied = 66.67% |
| | BEM/33.33% MAA by wt.), Rhodia |
| CAPB Surfactant | Chembetaine ™ CAD, Cocamidopropyl Betaine |
| | (amphoteric surfactant), Lubrizol Advanced |
| | Materials, Inc. (35% active) |
| CSEM | Visiomer ® Ethoxylated (25 moles) Cetearyl |
| | Methacrylate (as supplied = 75% CSEM/25% |
| | MAA by wt.), Evonik Industries. |
| | Bimax ™ Ethoxylated (25 moles) Cetearyl |
| | Methacrylate (as supplied = 93.75% |
| | CSEM/6.25% MAA by wt.), |
| Celvol ® 502 PVA | Polyvinyl Alcohol (hydrolysis % = 87-89%), |
| | Celanese Corpoartion |
| Diisopropyl Sebacate (INCI Name) | Schercemol ™ DIS Ester, Lubrizol Advanced |
| | Materials, Inc. |
| Dimethicone (and) Laureth-23 (and) C12-15 | Dow Corning ® 2-1491 Silicone Emulsion, Dow |
| Pareth-3 (INCI) | Corning Corporation |
| Disodium Laureth Sulfosuccinate (and) | Chemoryl ™ SFB-10K Surfactant Blend (30-34% |
| Sodium Cocoyl Isethionate (and) | active), Lubrizol Advanced Materials, Inc. |
| Cocamidopropyl Betaine | |
| DMDM Hydantoin (and) Iodopropyl | Glydant ™ Plus Preservative, Lonza |
| Butylcarbamate (INCI Name) | |
| EA | Ethyl Acrylate |
| EDGMA | Ethylene Glycol Dimethacrylate |
| Guar Hydroxypropyltrimonium Chloride | Jaguar ™ Excel Polymer, Rhodia |
| HEMA | 2-Hydroxyethyl Methacrylate |
| Isopropyl Isostearate (INCI Name) | Schercemol ™ 318 Ester, Lubrizol Advanced |
| | Materials, Inc. |
| MAA | Methacrylic Acid |
| MAMD | Methacrylamide |
| Methyl Chloro Isothiazolinone (and) Methyl | Kathon ™ CG Preservative, 5-chloro-2-methyl- |
| Isothiazolinone (INCI Name) | 4-isothiazolin-3-one (1.13%) and 2-methyl-4- |
| | isothiazolin-3-one (0.37%), Dow |
| Methyl Glucose Sequistearate (INCI Name) | Glucate ™ SS Emulsifier, Methyl Glucose |
| | Esterified With Stearic Acid, Lubrizol Advanced |
| | Materials, Inc. |
| Mica, Titanium Dioxide | Timiron ® Supersheen MP-1001, Pearlizing |
| | Agent, Merck KGaA |
| MPEG 350 MA | Bisomer ® 350 MA, Methoxy Polyethyleneglycol |
| | (8) Methacrylate, GEO Specialty Chemicals |
| NVP | N-Vinyl Pyrrolidone |
| PEG-20 Methyl Glucose Sequistearate | Glucamate SSE-20 ™ Emulsifier, Ethoxylated |
| (INCI Name) | (20 moles) Methyl Glucose Ether Esterified |
| | With Stearic Acid, Lubrizol Advanced |
| | Materials, Inc. |
| PEG200DA | Photomer ® 450 Polyethyleneglycol 200 |
| | Dimethacrylate, iGM Resins |
| 2-Phenoxyethanol | Microcare ™ PE Preservative, Thor Personal |
| | Care SAS. |
| Polyacrylate-2 Crosspolymer | Fixate Superhold Fixative Polymer, Copolymer |
| | of PEG/PPG-23/6 Dimethicone Citronate, C10-30 |
| | Alkyl PEG-25 Methacrylate and One or |
| | More Monomers of Acrylic Acid, Methacrylic |
| | Acid and One of Their Simple Esters, |
| | Crosslinked with Trimethylolpropane PEG-15 |
| | Triacrylate, Lubrizol Advanced Materials, Inc. |
| PVA | Selvol ™ 203 polyvinyl alcohol |
| SLES-2 Surfactant | Sulfochem ™ ES-2K, Sodium Laureth Sulfate- |
| | 2 moles of ethoxylation, Lubrizol Advanced |
| | Materials, Inc. (27.3% active) |
| SLS Surfactant | Sulfochem ™ Sodium Lauryl Sulfate (anionic |
| | surfactant), Lubrizol Advanced Materials, Inc. |
| | (30% active) |
| TBHP | t-butyl hydroperoxide |
| Titanium Dioxide/Glycrerin/Water | Covarine White WN 9787 Pigment, Sensient |
| | Cosmetic Technologies |
| TMPTA | Trimethylolpropane Triacrylate |
| VAC | Vinyl Acetate |
| VA-10 | Vinyl Decanoate |
| VA-086 | Azo VA-086 2,2′-Azobis[2-methyl-N-(2- |
| | hydroxyethyl)propionamide], Wako |

### Example 1

Monomer composition=EA/MAA/CSEM/AM*(60.5/34.5/5/1*)(wt. % total monomers)(*AM=1 wt. % based on total monounsaturated monomer wt.)

A HASE emulsion polymer was prepared as follows. A monomer premix was made by mixing 200 grams of deionized (D.I.) water, 5 grams of amphiphilic macromonomer, 7.17 grams of SLS, 33.33 grams of CSEM associative monomer (Visomer™ CSEM), 172.5 grams of MAA, 302.5 grams of EA. Initiator A was made by dissolving 0.32 grams of ammonium persulfate (APS) in 10 grams of D.I. water. Initiator B was prepared by dissolving 0.3 grams of APS in 75 grams of D.I. water with 4.27 grams of SLS. A 3-liter reactor vessel was charged with 550 grams of D.I. water, 6.67 grams of SLS, and then was heated to 85° C. under a nitrogen blanket with mild agitation. Initiator A was then added to the reactor. After 2.5 minutes, the monomer premix was metered into the vessel over a period of 120 minutes. Simultaneously, initiator B was metered into the reactor over a period of 120 minutes. The reaction temperature was maintained at 85° C. After completion of initiator B feed, the temperature of the contents of the reactor vessel was maintained at 85° C. for 60 minutes. The reactor content was then cooled to 49° C. A solution of 0.61 grams of 70% TBHP and 0.3 grams of SLS in 15 grams of D.I. water was added to the reactor. After 5 minutes, a solution of 0.59 grams of erythorbic acid in 15 grams of D.I. water was added to the reactor. The reactor content was maintained at 49° C. After 30 minutes, a solution of 0.64 grams of 70% TBHP and 0.29 grams of SLS in 15 grams of D.I. water was added to the reactor. After 5 minutes, a solution of 0.59 grams of erythorbic acid in 15 grams of D.I. water was added to the reactor. The reactor content was maintained at 49° C. for 30 minutes. The reactor content was cooled to the room temperature and then filtered through 100-micron cloth. The pH of the resulting emulsion was 2.7. The emulsion product had a polymer solids content of 34.8 wt. %, a viscosity 205 cps, and particle size 64 nm.

### Example 2 (Comparative)

Monomer composition=EA/MAA/CSEM(60.5/34.5/5)(wt. % total monomers)

An emulsion polymer was prepared same as in Example 1 except no amphiphilic macromonomer was used in polymerization reaction. The emulsion product had a polymer solids content of 32.2 wt. %, a viscosity of 32 cps and a particle size of 59 nm.

### Example 3

Monomer composition=EA/MAA/CSEM/AM*(64.5/34.5/1/1*)(wt. % total monomers)(*AM=1 wt. % based on total monounsaturated monomer wt.)

An emulsion polymer was prepared as in Example 1 except the monomer composition was changed to 64.5 wt. % EA, 34.5 MAA and 1 wt. % CSEM. The emulsion product had a polymer solids content of 34.2 wt. %, a viscosity of 60 cps and a particle size of 61 nm.

### Example 4

Monomer composition=EA/MAA/CSEM/AM*(63/34.5/2.5/1*)(wt. % total monomers)(*AM=1 wt. % based on total monounsaturated monomer wt.)

An emulsion polymer was prepared same as Example 1 except monomer compositions were changed to 63 wt. %, EA, 34.5 wt. % MAA and 2.5 wt. % CSEM. The emulsion product had a polymer solids content of 34.5%, a viscosity of 90 cps and a particle size of 60 nm.

### Example 5

Monomer composition=EA/MAA/BEM/AM*(60.5/34.5/5/1*)(wt. % total monomers)(*AM=1 wt. % based on total monounsaturated monomer wt.)

An emulsion polymer was prepared as in Example 1 except the monomer composition was changed to 60.5 wt. % EA, 34.5 wt. % MAA and 5 wt. % BEM (as supplied). The emulsion product had a polymer solids content of 31.3% wt. %, a viscosity of 143 cps and a particle size of 43 nm.

### Example 6 (Comparative)

Monomer composition=EA/MAA/BEM(60.5/34.5/5)(wt. % total monomers)

An emulsion polymer was prepared as in Example 5 except that no AM macromonomer was used in polymerization. The emulsion product had a polymer solids content of 30.1 wt. %, a viscosity of 24 cps and a particle size of 55 nm.

### Example 7

Monomer composition=EA/MAA/BEM/AM*(50/35/15/1*)(wt. % total monomers)(*AM=1 wt. % based on total monounsaturated monomer wt.)

An emulsion polymer was prepared as follows. A monomer premix was made by mixing 437.4 grams of D.I. water, 6 grams of AM macromonomer, 8.6 grams of SLS, 114.4 grams of BEM (as supplied), 199.8 grams of MAA, 285.6 grams of EA. Initiator A was made by dissolving 0.54 grams of ammonium persulfate APS in 14.4 grams of D.I. water. Initiator B was prepared by dissolving 0.54 grams of APS in 92.8 grams of D.I. water with 5.2 grams of SLS. A 3-liter reactor was charged with 702 grams of D.I. water, 9.2 grams of SLS, and then the contents were heated to 85° C. under a nitrogen blanket with mild agitation. Initiator A was then added to the reactor. After about 2-3 minutes, the monomer premix was metered into the reaction vessel over a period of 120 minutes. Simultaneously, initiator B was metered into the reactor over a period of 120 minutes. The reaction temperature was maintained at 85° C. After completion of initiator B feed, the temperature of the reaction vessel content was maintained at 85° C. for 60 minutes. The reactor content was cooled to 60° C. and a solution of 1.71 grams of 70% TBHP and 0.6 grams of SLS in 5.1 grams of D.I. water was added to the reactor. After 5 minutes, a solution of 1.38 grams of erythorbic acid in 18 grams of D.I. water was added to the reactor. The reactor content was maintained at 60° C. for about 30 minutes. Then, the reactor content was cooled to room temperature and filtered through 100-micron cloth. The pH of the resulting emulsion was 2.6. The emulsion had a solids 31.1 wt. %, a viscosity 16 cps, and particle size 75 nm.

### Example 8 (Comparative)

Monomer composition=EA/MAA/BEM/TMPTA*(50/35/15/0.3*)(wt. % total monomers)(*TMPTA=0.3 wt. % based on total monounsaturated monomer wt.)

A crosslinked emulsion polymer was prepared as in Example 7 except that the polymer was prepared with the conventional crosslinking monomer (TMPTA). In addition, no AM macromonomer was used in the polymerization medium. The emulsion product had a polymer solids content of 30.65 wt. %, a viscosity of 12 cps, and a particle size of 82 nm.

### Example 9 (Comparative)

Monomer composition=EA/MAA/BEM/TMPTA*(50/35/15/1*)(wt. % total monomers)(*TMPTA=0.3 wt. % based on total monounsaturated monomer wt.)

A crosslinked emulsion polymer was prepared as in Example 7 except that the polymer contained 1.0 wt % of conventional crosslinker (TMPTA). In addition, no AM macromonomer was used in polymerization medium. The emulsion product had a polymer solids content of 30.85 wt. %, a viscosity of 15 cps, and a particle size of 79 nm.

### Example 10

Monomer composition=EA/MAA/AA/BEM/AM*(50/30/5/15/1*)(wt. % total monomers)(*AM=1 wt. % based on total monounsaturated monomer wt.)

An emulsion polymer was prepared as in Example 7 except the monomer composition was changed to 50 wt. % EA, 30 wt. % MAA, 5 wt. % AA, 15 wt. % BEM (as supplied). The emulsion product had a polymer solids content of 30.6 wt. %, a viscosity of 12 cps, and a particle size of 86 nm.

### Example 11

Monomer composition=EA/MAA/AA/BEM/AM*(60.5/29.5/5/5/1*)(wt. % total monomers)(*AM=1 wt. % based on total monounsaturated monomer wt.)

An emulsion polymer was prepared as in Example 1 except the monomer composition was changed to 60.5 wt. %, EA, 29.5 wt. % MAA, 5 wt. % AA, 5 wt. % BEM (as supplied). The emulsion product had a polymer solids content of 31.4 wt. %, a viscosity of 23 cps, and a particle size of 66 nm.

### Example 12

Monomer composition=EA/MAA/AA/BEM/AM*(60.5/24.5/10/5/1*)(wt. % total monomers)(*AM=1 wt. % based on total monounsaturated monomer wt.)

An emulsion was prepared as in Example 1 except the monomer composition was changed to 60.5 wt. % EA/, 24.5 wt. % MAA, 10 wt. % AA, 5 wt. % BEM (as supplied). The emulsion product had a polymer solids content of 30.8 wt. %, a viscosity of 14 cps, and a particle size of 77 nm.

### Example 13

Monomer composition=EA/MAA/AA/BEM/MPEG 350 MA/AM*(55.5/29.5/5/5/5/1*)(wt. % total monomers)(*AM=1 wt. % based on total monounsaturated monomer wt.)

An emulsion polymer was prepared as in Example 1 except the monomer composition was changed to 55.5 wt. % EA, 29.5 MAA, 5 wt. % AA, 5 wt % BEM (as supplied), 5 wt. % MPEG 350 MA. The emulsion product had a polymer solids content of 31 wt. %, a viscosity of 18 cps, and a particle size of 70 nm.

### Example 14

Monomer composition=EA/MAA/BEM/AM*(50/38.75/11.25/0.7*)(wt. % total monomers)(*AM=0.7 wt. % based on total monounsaturated monomer wt.)

An emulsion polymer was prepared as follows. A monomer premix was made by mixing 240 grams of D.I. water, 4.2 grams of AM macromonomer, 8.6 grams SLS, 90 grams of BEM (as supplied), 232.5 grams MAA, 300 grams of EA. Initiator A was made by dissolving 0.54 grams of APS in 12 grams of D.I. water. Initiator B was prepared by dissolving 0.54 grams of APS in 92.8 grams of D.I. water with 5.2 grams of SLS. A 3-liter reactor was charged with 900 grams of D.I. water, 8 grams of SLS, and then the contents were heated to 85° C. under a nitrogen blanket with mild agitation. Initiator A was then added to the reactor. After 2.5 minutes, the monomer premix was metered into the reaction vessel over a period of 120 minutes. Similtaneously, initiator B was metered into the reactor over a period of 120 minutes. The reaction temperature was kept at 85° C. After completion of the initiator B feed, the temperature of the reaction vessel contents was maintained at 85° C. for 60 minutes. The reactor contents were then cooled to 49° C. A solution of 0.0.73 grams of 70% TBHP and 0.46 grams of SLS in 5.1 grams of D.I. water was added to the reactor. After 5 minutes, a solution of 0.7 grams of erythorbic acid in 9 grams of D.I. water was added to the reactor. The reactor contents were maintained at 49° C. After 30 minutes, a solution of 0.77 grams of 70% TBHP and 0.35 grams of 30% SLS in 5.1 grams of D.I. water was added to the reactor. After 5 minutes, a solution of 0.7 grams of erythorbic acid in 9 grams of D.I. water was added to the reactor. The reactor contents were maintained at 49° C. for about 30 minutes. The reactor contents were cooled to room temperature and filtered through 100-micron cloth. The pH of the resulting emulsion was 2.2. The emulsion had a polymer solids content of 30.6 wt. %, a viscosity of 15 cps, and a particle size 73 nm.

### Example 15 (Wt. % Total Monomers)

Monomer composition=EA/MAA/AA/CSEM/AM*(47.3/42/10.7/0.7*)(wt. % total monomers)(*AM=0.7 wt. % based on total monounsaturated monomer wt.)

An emulsion polymer was prepared as in Example 14 except the monomer composition was changed to 47.3 wt. % EA, 42.0 wt. % MAA, 10.7 wt. % CSEM (Bimax™ CSEM). The emulsion product had a polymer solids content of 31% wt. %, a viscosity of 18 cps, and a particle size of 70 nm.

### Example 16

Monomer composition=EA/BA/MAA/BEM/MPEG 350 MA/M (26/15/32.5/11.5/15/0.8*)(wt. % total monomers)(*AM=0.8 wt. % based on total monounsaturated monomer wt.)

An emulsion polymer was prepared as in Example 14 except the monomer composition was changed to 26 wt. % EA, 15 wt. % BA, 32.5 wt. % MAA, 11.5 wt. % BEM, 15 wt. % MPEG 350 MA and 0.8 wt. % AM macromonomer (based on total monounsaturated monomer wt.). The emulsion product had a polymer solids content of 31.4 wt. %, a viscosity of 23 cps, and a particle size of 70 nm.

### Example A

2.5 grams (100% active polymer solids) of the polymers of Examples 1 through 13 were formulated in a surfactant chassis containing 14 wt. % SLES, 3 wt. % CAPB (based on 100% active material), and DI water (q.s. to 100 wt. %). The pH of each of the polymer formulations was adjusted with an 18 wt. % (w/w) aqueous solution of NaOH as indicated in Table 1 below. Following base neutralization each of the pH adjusted formulations was evaluated for rheology and clarity properties in accordance with the protocol set forth in the test methodology above. The results are reported in Table 1 below.

TABLE 1

| | Polymer | | Yield | | | |
|---|---|---|---|---|---|---|
| Polymer | Emulsion | Formulation | Stress | Viscosity | | Transmittance |
| Ex. No. | pH | pH | (Pa) | (mPa · s) | NTU | (%) |
| 1 | 2.7 | 5.6 | 36.2 | 13640 | 35 | 53.6 |
| | | 6.3 | 17.0 | 7680 | 6.5 | 77.1 |
| 2 | 2.6 | 5.5 | 0 | 9160 | 3.4 | 91.6 |
| (Comparative) | | 6.1 | 0 | 3560 | 10.9 | 88.1 |
| 3 | 2.6 | 5.6 | 16.8 | 7000 | 8.3 | 69.6 |
| | | 6.6 | 12.9 | 4740 | 6.6 | 81.1 |
| 4 | 2.6 | 5.6 | 23.8 | 9460 | 10.6 | 57.2 |
| | | 6.6 | 10.3 | 4900 | 2.0 | 78.8 |
| 5 | 2.6 | 5.5 | 29.9 | 15820 | 8.7 | 82.8 |
| | | 6.3 | 11.0 | 9160 | 7.6 | 87.6 |
| 6 | 2.6 | 5.5 | 0 | 5840 | 3.3 | 90.0 |
| (Comparative) | | 6.5 | 0 | 1900 | 9.3 | 93.0 |
| 7 | 2.6 | 5.0 | 51.6 | 16800 | 13.1 | 43.7 |
| | | 5.98 | 20.1 | 10200 | 8.09 | 55.8 |
| 8 | 2.6 | 5.0 | 40.0 | 14760 | 20.5 | 49.6 |
| | | 6.0 | 6.0 | 6240 | 4.71 | 78.8 |
| 9 | 2.5 | 4.9 | 42.1 | 14020 | 34.8 | 25.4 |
| | | 5.9 | 20.1 | 8200 | 21.4 | 39.0 |
| 10 | 2.5 | 5.0 | 49.5 | 16640 | 15.4 | 49.8 |
| | | 6.0 | 13.0 | 9020 | 2.21 | 88.6 |
| 11 | 2.5 | 5.0 | 25.6 | 9940 | 8.8 | 69.9 |
| | | 6.0 | 13.7 | 6360 | 3.96 | 85.5 |
| 12 | 2.5 | 5.0 | 31.9 | 10880 | 11.3 | 44.3 |
| | | 6.0 | 12.9 | 6260 | 2.54 | 89.0 |
| 13 | 2.5 | 5.0 | 25.8 | 9140 | 9.69 | 61.1 |
| | | 6.1 | 10.6 | 5260 | 3.07 | 89.1 |

### Example 17

Monomer composition=EA/MAA/n-BA/HEMA/BEM/AM*(35/30/15/15/5/1*)(wt. % total monomers)(*AM=1 wt. % based on total monounsaturated monomer wt.)

An emulsion polymer was prepared as follows. A monomer premix was made by mixing 140 grams of D.I. water, 5 grams of AM macromonomer, 16.67 grams of SLS, 175 grams of EA, 75 grams of n-BA, 150 grams of MAA, 75 grams of HEMA and 33.3 grams of BEM (as supplied). Initiator A was made by dissolving 5 grams of Azo VA-086 in 40 grams of D.I. water. Initiator B was prepared by dissolving 2.5 grams of Azo VA-086 in 100 grams of D.I. water. A 3-liter reactor was charged with 800 grams of D.I. water, 6.67 grams of SLS and 10 grams of PVA. The contents of the reactor were heated to 87° C. under a nitrogen blanket with mild agitation. After holding the reactor contents at 87° C. for one hour, initiator A was added to the reactor. After about 1 minute, the monomer premix was metered into the reaction vessel over a period of 120 minutes. About 3 minutes after the start of monomer premix metering, initiator B was metered into the reactor over a period of 150 minutes. The reaction temperature was maintained at 87° C. After completion of the initiator B feed, the temperature of the reactor contents was maintained at 87° C. for 60 minutes. The reactor contents were then cooled to 49° C. A solution of 0.61 grams of 70% TBHP and 0.38 grams of 3 SLS in 15 grams of D.I. water was added to the reactor. After 5 minutes, a solution of 0.59 grams of erythorbic acid in 15 grams of D.I. water was added to the reactor. The reactor contents were maintained at 49° C. After 30 minutes, a solution of 0.64 grams of 70% TBHP and 0.29 grams of SLS in 15 grams of D.I. water was added to the reactor. After 5 minutes, a solution of 0.59 grams of erythorbic acid in 15 grams of D.I. water was added to the reactor. The reactor contents were maintained at 49° C. for about 30 minutes. The reactor contents were cooled to the room temperature and filtered through 100-micron cloth. The pH of the resulting emulsion was 3.1. The emulsion had a polymer solids content of 30.7%, a viscosity of 155 cps, and a particle size of 82 nm.

### Examples 18-28

The HASE polymers of Examples 18-28 were prepared as in Example 17 except that the surfactant type and amount, the amount of PVA protective colloid and the monomer components were changed as shown in Table 2 below.

TABLE 2

| | | SLS | | | | | | | | |
|---|---|---|---|---|---|---|---|---|---|---|
| | | Charged | | | | | | | | |
| Ex. | SLS in | to | | EA | MAA | n-BA | HEMA | BEM | AM¹ | P.S.² |
| No. | Premix¹ | Reactor¹ | PVA¹ | (wt. %) | (wt. %) | (wt. %) | (wt. %) | (wt. %) | (wt. %) | (wt. %) |
| 18 | 1 | 0.4 | 2 | 35 | 30 | 15 | 15 | 5 | 1 | 30.7 |
| 19 | 0.5 | 0.4 | 2 | 35 | 10 | 15 | 35 | 5 | 1 | 31.0 |
| 20 | 0.5 | 0.4 | 2 | 35 | 20 | 15 | 25 | 5 | 1 | 31.3 |
| 21 | 1 | 0.4 | 2 | 45 | 35 | 15 | 0 | 5 | 1 | 31.3 |
| | (AOS) | (AOS) | | | | | | | | |
| 22 | 1 | 0.4 | 2 | 45 | 35 | 15 | 0 | 5 | 1 | 31.3 |
| 23 | 1 | 0.1 | 0 | 45 | 35 | 15 | 0 | 5 | 1 | 31.1 |
| 24 | 1 | 0.1 | 0 | 40 | 40 | 15 | 0 | 5 | 1 | 31.2 |
| 25 | 1 | 0.4 | 2 | 35 | 5 | 15 | 40 | 5 | 1 | 31.3 |
| 26 | 1 | 0.4 | 2 | 35 | 35 | 15 | 10 | 5 | 1 | 31.6 |
| 27 | 1 | 0.4 | 2 | 35 | 40 | 15 | 5 | 5 | 1 | 31.8 |
| 28 | 1 | 0.4 | 2 | 35 | 45 | 15 | 0 | 5 | 1 | 32.0 |

¹Parts by wt. (100% active material) per 100 parts by wt. (100% active material) of total monounsaturated monomers.
²P.S. = Polymer solids in emulsion product

### Example B

2.5 grams (100% active) of the polymers of Examples 18 through 28 were formulated in a surfactant chassis containing 14 wt. % SLES, 3 wt. % CAPB (based on 100% active material), and DI water (q.s. to 100 wt. %). The pH of each of the polymer formulations was adjusted with an 18 wt. % (w/w) aqueous solution of NaOH as indicated in Table 3 below. Following base neutralization each of the pH adjusted formulations was evaluated for rheology and clarity properties in accordance with the protocol set forth in the test methodology above. The results are reported in the Table below.

TABLE 3

| | Polymer | | Yield | | | Trans- |
|---|---|---|---|---|---|---|
| Polymer | Emulsion | Formulation | Stress | Viscosity | | mittance |
| Ex. No | pH | pH | (Pa) | (mPa · s) | NTU | (%) |
| 18 | 3.0 | 5.8 | 20.9 | 9360 | 13.8 | 71.8 |
| | | 6.4 | 10.0 | 5100 | 7.7 | 84.4 |
| 19 | 3.2 | 5.6 | 16.4 | 6360 | 17.3 | 51.6 |
| | | 6.9 | 7.1 | 3240 | 12.3 | 70.8 |
| 20 | 3.2 | 5.6 | 27.8 | 11580 | 17.3 | 56.3 |
| | | 6.4 | 10.0 | 4500 | 8.4 | 69.4 |
| 21 | 2.8 | 5.5 | 24.7 | 11140 | 5.5 | 61.2 |
| | | 6.3 | 12.4 | 5720 | 10.5 | 74.5 |
| 22 | 2.6 | 5.3 | 24.7 | 10420 | 13.7 | 61.0 |
| | | 6.3 | 13.8 | 6940 | 10.0 | 68.7 |
| 23 | 2.3 | 5.4 | 29.4 | 12360 | 13.8 | 64.7 |
| | | 6.4 | 14.1 | 7780 | 3.7 | 93.3 |
| 24 | 2.2 | 5.7 | 23.0 | 10140 | 16.9 | 57.5 |
| | | 6.4 | 13.8 | 8260 | 4.3 | 74.4 |
| 25 | 3.6 | 5.9 | 14.2 | 5740 | 16.0 | 56.0 |
| | | 6.5 | 8.2 | 3940 | 12.4 | 68.4 |
| 26 | 3.1 | 5.5 | 24.1 | 8500 | 19.5 | 43.4 |
| | | 6.5 | 12.7 | 8040 | 3.7 | 85.4 |
| 27 | 3.1 | 5.4 | 22.4 | 7640 | 19.5 | 38.4 |
| | | 6.3 | 10.6. | 6220 | 6.3 | 89.2 |
| 28 | 3.0 | 5.4 | 21.5 | 7160 | 33.4 | 27.8 |
| | | 6.7 | 5.6 | 7620 | 5.8 | 80.2 |

### Example 29

Monomer composition=EA/MAA/n-BA/BEM/AM*/APE*(45/35/15/5/1*/0.03*)(wt. % total monomers)(*AM and *APE=1 wt. % and 0.03 wt. %, respectively, based on total monounsaturated monomer wt.)

A crosslinked emulsion polymer was prepared as follows. A monomer premix was made by mixing 140 grams of DI water, 5 grams of AM macromonomer, 16.67 grams of SLS, 225 grams of EA, 75 grams of n-BA, 175 grams of methacrylic acid MAA, 33.3 grams of BEM (as supplied) and 1.5 grams of APE. Initiator A was made by dissolving 5 grams of Azo VA-086 in 40 grams of DI water. Initiator B was prepared by dissolving 2.5 grams of Azo VA-086 in 100 grams of DI water. A 3-liter reactor was charged with 800 grams of DI water and 6.67 grams of SLS. The contents of the reactor were heated to 87° C. under a nitrogen blanket with mild agitation. When the reactor contents reached 87° C. initiator A was added to the reactor. After about 2-3 minutes, the monomer premix was metered into the reaction vessel over a period of 120 minutes. Simultaneously, initiator B was metered into the reactor a period of 150 minutes. The reaction temperature was maintained at 87° C. After completion of the initiator B feed, the temperature of the reactor contents was maintained at 87° C. for 60 minutes. The reactor contents were then cooled to 49° C. A solution of 0.61 grams of 70% TBHP and 0.38 grams of SLS in 15 grams of DI water was added to the reactor. After 5 minutes, a solution of 0.59 grams of erythorbic acid in 15 grams of DI water was added to the reactor. The reactor contents were maintained at 49° C. After 30 minutes, a solution of 0.64 grams of 70% TBHP and 0.29 grams of SLS in 15 grams of DI water was added to the reactor. After 5 minutes, a solution of 0.59 grams of erythorbic acid in 15 grams of DI water was added to the reactor. The reactor was maintained at 49° C. for about 30 minutes. The reactor was cooled to the room temperature and filtered through 100-micron cloth. The pH of the resulting emulsion was 2.4. The emulsion had a polymer solids content of 30.7 wt. %, a viscosity of 22 cps, and a particle size of 79 nm.

### Examples 30-32

The HASE polymers of Examples 30-31 were prepared as in Example 29 except that the surfactant type and amount, the amount of PVA protective colloid and the monomer components were changed as shown in Table 4 below.

TABLE 4

| | | EA | | n-BA | | X- | | |
|---|---|---|---|---|---|---|---|---|
| Ex. | SLS in | (wt. | MAA | (wt. | BEM | Linker¹ | | |
| No. | Premix¹ | %) | (wt. %) | %) | (wt. %) | (wt. %) | AM¹ | P.S.² |
| 30 | 1 | 45 | 35 | 15 | 5 | 0.1³ | 1 | 31.1 |
| 31 | 0.5 | 45 | 35 | 15 | 5 | 0.3² | 1 | 31.3 |
| 32 | 0.5 | 45 | 35 | 15 | 5 | 0.1⁴ | 1 | 30.6 |

¹Parts by wt. (100% active material) per 100 parts by wt. (100% active material) of total monounsaturated monomers.
²P.S. = Polymer solids in emulsion product
³EGDMA
⁴PEG200DA

### Example C

2.5 grams (100% active) of the polymers of Examples 30 through 32 were formulated in a surfactant chassis containing 14 wt. % SLES, 3 wt. % CAPB (based on 100% active material), and DI water (q.s. to 100 wt. %). The pH of each of the polymer formulations was adjusted with an 18 wt. % (w/w) aqueous solution of NaOH as indicated in Table 5 below. Following base neutralization each of the pH adjusted formulations was evaluated for rheology and clarity properties in accordance with the protocol set forth in the test methodology above. The results are reported in the Table below.

TABLE 5

| | Polymer | | Yield | | | Trans- |
|---|---|---|---|---|---|---|
| Polymer | Emulsion | Formulation | Stress | Viscosity | | mittance |
| Ex. No | pH | pH | (Pa) | (mPa · s) | NTU | (%) |
| 30 | 2.5 | 5.4 | 20.0 | 13200 | 28.7 | 50.7 |
| | | 6.2 | 16.0 | 7660 | 20.5 | 65.0 |
| 31 | 2.4 | 5.4 | 26.7 | 15600 | 36.7 | 44.2 |
| | | 6.1 | 19.9 | 8260 | 34.0 | 52.9 |
| 32 | 2.4 | 5.5 | 30.6 | 11740 | 22.3 | 55.0 |
| | | 5.9 | 22.4 | 9280 | 54.4 | 21.2 |

### Example 33

Monomer composition=EA/MAA/BEM/NVP/AM*(50/29.7/15/5.3/1*)(wt. % total monomers)(*AM=1 wt. % based on total monounsaturated monomer wt.)

An emulsion polymer was prepared as follows. A monomer premix was made by mixing 137.4 grams of DI water, 6 grams of amphiphilic macromonomer, 8.6 grams of SLS, 30%, 30 grams of NVP, 285.6 grams of EA, 169.8 grams of MAA and 114.4 grams of BEM (as supplied). Initiator No. 1 was made by mixing 0.54 grams of APS in 14.4 grams of DI water. Initiator No. 2 was prepared by dissolving 0.54 grams of APS and 5.2 grams of SLS in 87.6 grams of DI water. A 3-liter reactor vessel was charged with 702 grams of DI water and 9.2 grams of SLS, and the contents were heated to 85° C. under a nitrogen blanket and mild agitation. Initiator No. 1 was added to the reaction vessel. After about 1 minute, the monomer premix was metered into the reactor vessel over a period of 120 minutes. Simultaneously, initiator No. 2 was metered into the reactor vessel over a period of 120 minutes as well. After the completion of monomer premix feed, 18 grams of DI water was added to flush the residual monomers from the premixer into the reactor. The temperature of the reactor contents was maintained at 85° C. for 60 minutes. The reactor contents were then cooled to 49° C. A solution of 0.77 grams of 70% TBHP, 0.468 grams of SLS and 5.1 grams of DI water was added to the reactor vessel. After 5 minutes, a solution of 0.7 grams of erythorbic acid in 9 grams of DI water was added to the reactor vessel. After 30 minutes, the amounts of TBHP solution and erythobic acid solution as added immediately above were introduced into the reactor vessel. The contents of the reactor vessel were maintained at 49° C. for about 30 minutes. Then, the contents of the reactor vessel were cooled to room temperature and filtered through 100-micron cloth. The emulsion had a polymer solids content of 36 wt. %.

### Example 34

Monomer composition=EA/MAA/BEM/n-VP/VAC/AM*(44.7/29.7/15/5.3/5.3/1*)(wt. % total monomers)(*AM=1 wt. % based on total monounsaturated monomer wt.)

An emulsion polymer was prepared as follows. A monomer premix was made by mixing 137.4 grams of DI water, 6 grams of amphiphilic macromonomer, 8.6 grams of SLS, 30 grams of NVP, 30 grams of VAC, 255.6 grams of EA, 169.8 grams of MAA and 114.4 grams of BEM (as supplied). Initiator No. 1 was made by mixing 0.54 grams of APS in 14.4 grams of DI water. Initiator No. 2 was prepared by dissolving 0.54 grams of APS and 5.2 grams of SLS in 87.6 grams of DI water. A 3-liter reactor vessel was charged with 702 grams of DI water and 9.2 grams of SLS, and then the reactor contents were heated to 85° C. under a nitrogen blanket and mild agitation. Initiator No. 1 was then added to the reaction vessel. After about 1 minute, the monomer premix was metered into the reaction vessel over a period of 120 minutes, while at the same time initiator No. 2 was metered into the reaction vessel over a period of 120 minutes. After the completion of monomer premix feed, 18 grams of DI water was added to flush the residual monomers from the premixer into the reactor. The temperature of the reactor contents was maintained at 85° C. for 60 minutes. The reactor contents were then cooled to 49° C. A solution of 0.77 grams of 70% TBHP, 0.468 grams of SLS and 5.1 grams of DI water was added to the reaction vessel. After 5 minutes, a solution of 0.7 grams of erythorbic acid in 9 grams of DI water was added to the reaction vessel. After 30 minutes, the same amount of TBHP solution and erythobic acid solution as previously added immediately above were introduced into the reaction vessel. The reactor contents were maintained at 49° C. for about 30 minutes. Then, the reactor contents were cooled to room temperature and filtered through 100-micron cloth. The emulsion had a polymer solids content of 36 wt. %.

### Example 35

Polymers of the disclosed technology were formulated in a low concentration surfactant chassis (total surfactant concentration of 6 wt. %: 5 wt. % SLES-2 and 1 wt. % CAPB active wt. %) and evaluated for rheology, clarity properties and bead suspension stability. Two commercially available rheology modifiers, as well as two comparative polymers which were not prepared from an amphiphilic macromonomer, were identically formulated and evaluated. The formulation components and amounts are identified in Table 6 and the test results are reported in Table 7.

TABLE 6

| | | Amount | |
|---|---|---|---|
| | Component | (wt. %) | Function |
| 1 | DI Water | q.s. to 100 | Diluent |
| 2 | Test Polymer | See Table 7 | Rheology Modifier |
| 3 | SLES-2 (27.3% active) | 18.31 | Detersive Anionic |
| | | (5 wt. % actives) | Surfactant |
| 4 | CAPB (35 wt. % active) | 2.86 | Amphoteric |
| | | (1 wt. % actives) | Surfactant |
| 5 | NaOH (18 wt. % | q.s. to pH ≈ 6.5 | pH adjusting agent |
| | aqueous w/w) | | |
| 6 | Kathon ™ CG Preservative | 0.05 | Antimicrobial |
| 7 | Fragrance (Rose | 1.0 | Fragrance |
| | Petals Oil)¹ | | |

¹Lebermuth Company, Inc.

Formulation Procedure:

1. Add polymer to DI water with gentle mixing to form a homogeneous dispersion.

2. Add surfactants to the dispersion obtained in step 1 in the order listed in Table 6 and mix until uniform.

3. Neutralize the surfactant containing mixture obtained in step 2 with 18% NaOH to a pH of approximately 6.5.

4. Add preservative and fragrance to the neutralized formulation obtained in step 3 and mix until uniform

5. Measure Brookfield viscosity, turbidity and bead suspension stability. The results are presented in Table 7.

TABLE 7

| | Aculyn ™ 38 | Aculyn ™ 88 | Aculyn ™ 88 | Aculyn ™ 88 | | | | | | | |
|---|---|---|---|---|---|---|---|---|---|---|---|
| Polymer | ASE | HASE | HASE | HASE | Ex. 7 | Ex. 8¹ | Ex. 9¹ | Ex. 11 | Ex. 15 | Ex. 33 | Ex. 34 |
| Polymer | 2 | 1.6 | 1.75 | 2 | 2 | 2 | 2 | 2 | 2 | 2 | 2 |
| (wt. % | | | | | | | | | | | |
| active) | | | | | | | | | | | |
| PH | 6.59 | 6.66 | 6.65 | 6.67 | 6.40 | 6.64 | 6.55 | 6.47 | 6.40 | 6.61 | 6.50 |
| Turbidity | 135.0 | 26.0 | 22.3 | 21.8 | 8.65 | 5.02 | 17.2 | 11.7 | 8.36 | 6.74 | 9.21 |
| (NTU) | | | | | | | | | | | |
| Viscosity | 2110 | 26400 | 44660 | 81400 | 5990 | 8780 | 5140 | 3250 | 6490 | 9680 | 9980 |
| (mPa · s) | | | | | | | | | | | |
| Bead | Pass | Fail | Pass | Pass | Pass | Fail | Fail | Pass | Pass | Pass | Pass |
| Suspension | | | | | | | | | | | |
| Stability | | | | | | | | | | | |
| (3 months | | | | | | | | | | | |
| at 45° C.) | | | | | | | | | | | |

¹Comparative

At low surfactant concentrations the polymers of the present technology prepared from an amphiphilic macromonomer provide bead suspension stability, clarity and ideal viscosity values as compared to a commercially available ASE and HASE polymer and the conventionally crosslinked polymers (prepared from substantially similar monomers but without the amphiphilic macromonomer).

### Example 36

Polymers of the disclosed technology were formulated in a medium concentration surfactant chassis (total surfactant concentration of 10 wt. %: 8 wt. % SLES-2 and 2 wt. % CAPB active wt. %) and evaluated for clarity, rheology and suspension stability properties at two different pH values (no base neutralization vs. base neutralization). The formulation components and amounts are identified in Table 8 and the test results are reported in Table 9.

TABLE 8

| | | Amount (wt. %) | | |
|---|---|---|---|---|
| | | pH | pH | |
| | | No Base | Base | |
| | Components | Neutralization | Neutralized | Function |
| 1 | DI Water | 56.85 | 56.85 | Diluent |
| 2 | Tetrasodium EDTA | 0.05 | 0.05 | Chelating Agent |
| 3 | Polymer (100% active) | 2.5 | 2.5 | Rheology Modifier |
| 4 | SLES-2 (27.3% active) | 29.3 | 29.3 | Detersive Anionic Surfactant |
| 5 | CAPB (35% active) | 5.71 | 5.71 | Amphoteric Surfactant |
| 6 | Kathon ™ CG Preservative | 0.05 | 0.05 | Antimicrobial |
| 7 | NaOH | 0 | q.s. to pH ≈ | pH Adjusting Agent |
| | (18 wt. % aqueous w/w) | | 6.5 | |
| 8 | DI Water | q.s. to 100 | q.s. to 100 | Diluent |

Procedure:

1. Dissolve chelating agent in DI water with mixing.

2. Add polymer to the step 1 solution with mixing.

3. Add surfactants to the mixture obtained in step 2 in the order listed in Table 8 and mix until uniform.

4. To prepare the based neutralized test sample, add 18% NaOH to the mixture obtained in step 3 with continuous mixing to neutralize the composition to a pH of about 6.5.

5 Add DI water to bring the composition to 100% and mix until homogeneous.

TABLE 9

| | | Polymer No. | | | |
|---|---|---|---|---|---|
| | | Ex. 7 | Ex. 11 | Ex. 14 | Ex. 16 |
| As is | pH (no NaOH) | 5.3 | 5.2 | 5.3 | 5.4 |
| Formulation | Turbidity (NTU) | 40.8 | 25.6 | 28.3 | 20.5 |
| (no base | Viscosity | 9847 | 6123 | 7712 | 8459 |
| neutralization) | (mPa · s) | | | | |
| | Bead Suspension | Pass | Pass | Pass | Fail |
| | Stability | | | | |
| | (3 months at | | | | |
| | 45° C.) | | | | |
| Neutralized | pH (NaOH | 6.6 | 6.5 | 6.5 | 6.8 |
| Formulation | added) | | | | |
| (base | Turbidity (NTU) | 19.4 | 16.3 | 9.7 | 7.9 |
| neutralization) | Viscosity | 26390 | 8593 | 16720 | 33120 |
| | (mPa · s) | | | | |
| | Bead Suspension | Pass | Pass | Fail | Fail |
| | Stability | | | | |
| | (3 months at | | | | |
| | 45° C.) | | | | |

At medium surfactant concentrations the polymers of the present technology prepared from an amphiphilic macromonomer provide bead suspension stability, clarity and ideal viscosity values. The results indicate that no base neutralization is necessary to obtain clarity, ideal viscosity properties and stable suspension of particulate materials. However, increasing the pH (polymer neutralization) enhances the clarity and viscosity properties of the surfactant composition in which the polymer is included.

### Example 37

Polymers of the disclosed technology were formulated in a highly concentrated surfactant chassis (total surfactant concentration of 18 wt. %: 16 wt. % SLES-2 and 2 wt. % CAPB active wt. %) and evaluated for clarity, rheology and suspension stability properties at two different pH values and polymer concentrations (no base neutralization vs. base neutralization). The formulation components and amounts are identified in Table 10 and the test results are reported in Table 11.

TABLE 10

| | | Amount (wt. %) | | |
|---|---|---|---|---|
| | | No Base | Base | |
| | Components | Neutralization | Neutralization | Function |
| 1 | DI Water | 24.15 | 20.00 | Diluent |
| 2 | Tetrasodium EDTA | 0.05 | 0.05 | Chelating Agent |
| 3 | Polymer (100% active) | 2 | 3 | Rheology Modifier |
| 4 | SLES-2 (27.3% active) | 58.61 | 58.61 | Detersive Anionic Surfactant |
| 5 | CAPB (35% active) | 5.71 | 5.71 | Amphoteric Surfactant |
| 6 | Kathon ™ CG Preservative | 0.05 | 0.05 | Antimicrobial |
| 7 | NaOH | 0 | q.s. to pH ≈ | pH Adjusting Agent |
| | (18 wt. % aqueous w/w) | | 6.5 | |
| 8 | DI Water | 5 | q.s. to 100 | Diluent |

The procedure utilized to formulate the compositions of Example 36 were repeated in the present example.

TABLE 11

| | As is Formulation | | | Neutralized Formulation |
|---|---|---|---|---|
| | (no base neutralization) | | | (base neutralized) |
| | Polymer No. | | | |
| | Ex. 7¹ | Ex. 11¹ | Ex. 15¹ | Ex. 11² |
| pH | 5.5 | 5.4 | 5.5 | 6.5 |
| Turbidity (NTU) | 16.1 | 9.9 | 16.5 | 4.1 |
| Viscosity³ | 11720 | 11050 | 13510 | 27530 |
| (mPa · s) | | | | |
| Bead | Pass | Pass | Pass | Pass⁴ |
| Suspension | | | | |
| Stability | | | | |
| (3 months at | | | | |
| 45° C.) | | | | |

¹2 wt. % active polymer solids.
²3 wt. % active polymer solids.
³Viscosity measured by AR-G2 Rheometer, TA Instruments.
⁴Only able to stabilize Type 3 beads.

At higher surfactant concentrations the polymers of the present technology prepared from an amphiphilic macromonomer provide bead suspension stability, clarity and ideal viscosity values. The results indicate that no base neutralization is necessary to obtain clarity, ideal viscosity properties and stable suspension of particulate materials. However, increasing the pH (polymer neutralization) enhances the clarity and viscosity properties of the surfactant composition in which the polymer is included.

### Example 38 (Pearlized Conditioning Shampoo)

Two pearlized conditioning shampoo compositions containing a surfactant chassis comprising 13 active wt. % SLES-2 and 2 active wt. % CAPB were separately formulated with the HASE polymers of Examples 11 and 15. The formulation components are set forth in Table 12.

TABLE 12

| | | Amount | |
|---|---|---|---|
| | Component | (wt. %) | Function |
| 1 | DI Water | 34.37 | Diluent |
| 2 | Tetrasodium EDTA | 0.05 | Chelating Agent |
| 3 | SLES-2 (27.3% active) | 47.62 | Detersive Anionic Surfactant |
| 4 | Polymer | 1.5 (active | Rheology Modifier |
| | | solids) | |
| 5 | Mica, Titanium Dioxide | 0.2 | Pearlizing Agent |
| 6 | CAPB (35% active) | 5.71 | Amphoteric Surfactant |
| 7 | Dimethicone (and) | 2 | Conditioning Silicone |
| | Laureth-23 (and) C12-15 | | |
| | Pareth-3 | | |
| 8 | Guar | 5 | Cationic Conditioning |
| | Hydroxypropyltrimonium | | Polymer |
| | Chloride, Jaguar ® excel | | |
| | (2% solution) | | |
| 9 | Fragrance | 0.5 | Fragrance |
| 10 | Kathon ™ CG | 0.05 | Antimicrobial |
| | Preservative | | |

Procedure:

1. Dissolve chelating agent in DI water under mild mixing.

2. Add components 3 to 10 one at a time in the listed order to the mixture prepared in step 1. Insure that each component is homogenously dispersed in the mixture before adding the successive component. After the last component is added continue mixing until uniform.

The pearlized conditioning shampoo compositions were evaluated for viscosity properties and the ability to stability suspend the pearlizing agent in accelerated suspension stability testing. The results are set forth in Table 13. The results indicate that no base neutralization is necessary to obtain ideal viscosities and the stable suspension of particulate materials (pearlizing agent). In addition, the tested polymers showed good compatibility with the cationic polymer, fragrance and silicone emulsion.

TABLE 13

| Polymer | Ex. 11 | Ex. 15 |
|---|---|---|
| pH | 5.62 | 5.63 |
| Viscosity (mPa · s) | 4490 | 4680 |
| Bead Suspension | Pass | Pass |
| Stability | | |
| (4 wks. at 45° C.) | | |

### Example 39 (Body Wash Containing Soybean Oil)

Two body wash compositions containing a surfactant chassis comprising 4.3 active wt. % ALS, 6.7 active wt. % ALES-3 and 2.5 active wt. % CAPB were separately formulated with the HASE polymers of Examples 14 and 16. The formulation components are set forth in Table 14.

TABLE 14

| | | | Amount | |
|---|---|---|---|---|
| Part | | Component | (wt. %) | Function |
| A | 1 | DI Water | 22.30 | Diluent |
| | 2 | Tetrasodium EDTA | 0.05 | Chelating Agent |
| | 3 | ALS (29.1% active) | 14.78 | Surfactant |
| | 4 | ALES-3 (26.1 wt. % active) | 25.67 | Surfactant |
| B | 5 | Soybean Oil (U.S.P.) | 15.00 | Skin Conditioner |
| | 6 | Polymer (100% active) | 1.75 | Rheology Modifier |
| C | 7 | NaOH (18 wt. % | q.s. | pH adjusting agent |
| | | aqueous w/w) | to pH | |
| D | 8 | Guar | 5.00 | Cationic Skin |
| | | Hydroxypropyltrimonium | | Conditioner |
| | | Chloride, Jaguar ® excel (2% | | |
| | | solution) | | |
| | 9 | Glycerin (U.S.P.) | 5.00 | Humectant |
| | 10 | CAPB (35% active) | 5.00 | Surfactant |
| | 11 | Kathon ™ CG Preservative | 0.05 | Antimicrobial |

Procedure:

1. Combine Part A components and mix until uniform.

2. Add Part B components to Part A one at a time in the order listed with good mixing between additions. Mix until uniform.

3. Add NaOH solution to neutralize the AB mixture and mix until uniform.

4. Add Part D components to the ABC mixture one at a time in the order listed with good mixing between additions. Mix the components until uniform.

The body wash compositions were evaluated for viscosity properties and the ability to provide stable emulsions with the soy bean oil in accelerated stability testing. The results are set forth in Table 15. The results indicate that ideal viscosities and stable emulsions are obtained. In addition, the tested polymers showed good compatibility with the cationic skin conditioning polymer.

TABLE 15

| Polymer | Ex. 14 | Ex. 16 |
|---|---|---|
| pH | 6.56 | 7.08 |
| Viscosity (mPa · s) | 21690 | 15680 |
| Emulsion Stability | Pass | Pass |
| (4 wks. at 45° C.) | | |

### Example 40 (Clear Hair Styling Gel)

Hair styling gels containing two commercially available hair fixative polymers were separately formulated with the HASE polymers of Examples 11, 14 and 15. The formulation components are set forth in Tables 16 and 17. The tested HASE rheology modifier polymers had excellent compatibility with the fixative polymers, demonstrating good viscosity profiles, excellent clarity and freeze-thaw stability.

Procedure:

1. Thoroughly mix rheology modifier in DI water.

2. Add fixative polymer to the mixture obtained in step 1 and mix until uniform.

3. Add pH adjusting agent to the mixture obtained in step 2 and mix until uniform.

4. Add the preservative to the mixture obtained in step 3 and mix until uniform.

TABLE 16

| | | Amount (wt. %) | | | |
|---|---|---|---|---|---|
| | | Example Polymer | | | |
| | Components | Ex. 11 | Ex. 14 | Ex. 15 | Function |
| 1 | DI Water | 88.5 | 89.6 | 89.6 | Diluent |
| 2 | Polymer (100 active wt. %) | 0.93 | 0.62 | 0.62 | Rheology Modifier |
| 3 | Acrylates Crosspolymer-3 (31% | 7 | 7 | 7 | Hair Fixative Polymer |
| | active) | | | | |
| 4 | Aminomethyl Propanol (95%) | 1.1 | 1 | 1 | pH Adjusting Agent |
| 5 | 2-Phenoxyethanol | 0.4 | 0.4 | 0.4 | Preservative |

Properties

| | | | |
|---|---|---|---|
| pH | 7.31 | 7.05 | 7.34 |
| Viscosity (mPa · s) | 19070 | 22750 | 20000 |
| Clarity (% T at 420 nm) | 90.1 | 88.2 | 81.1 |
| Freeze/Thaw Stability (3 cycles) | Pass | Pass | Pass |

TABLE 17

| | | Amount (wt. %) | | | |
|---|---|---|---|---|---|
| | | Example Polymer | | | |
| | Components | Ex. 11 | Ex. 14 | Ex. 15 | Function |
| 1 | DI Water | 89.5 | 90.1 | 90.1 | Diluent |
| 2 | Polymer (100 active wt. %) | 0.62 | 0.47 | 0.47 | Rheology Modifier |
| 3 | Polyacrylate-2 Crosspolymer | 7 | 7 | 7 | Hair Fixative Polymer |
| | (30.5 active) | | | | |
| 4 | Aminomethyl Propanol (95%) | 1.1 | 1 | 1 | pH Adjusting Agent |
| 5 | 2-Phenoxyethanol | 0.4 | 0.4 | 0.4 | Preservative |

Properties

| | | | |
|---|---|---|---|
| pH | 6.87 | 7.265 | 7.28 |
| Viscosity (mPa · s) | 24470 | 19030 | 14390 |
| Clarity (% T at 420 nm) | 89.2 | 82.5 | 77.2 |
| Freeze/Thaw Stability (3 cycles) | Pass | Pass | Pass |

### Example 41 (Light Foundation Cream)

The polymer of Example 11 was used to formulate a light foundation cream utilizing the components in Table 18. The rheology modifier of Example 11 provided a stable emulsion having a creamy consistency as well as providing for the stable suspension of pigment particles.

Procedure:

1. Thoroughly mix Part A components and heat to 50° C.

2. Under continuous mixing, slowly add Part B pigment dispersion into Part A. Mix until uniform and heat to 75° C.

3. In a separate vessel mix Part C components until uniform and heat to 75° C.

4. Add Part C to the AB batch with good agitation and mix until homogeneous. Maintain the temperature at 75° C. and homogenize the ABC batch at 6500 rpm using a VWR VDI 25 Homogenizer.

5. Remove heat, when batch cools to 60-70° C. add component 8 (polymer) to homogenized batch and slowly mix until polymer is uniformly dispersed.

6. When the temperature of the batch cools to 50-60° C., add component 9 (triethanolamine) to the batch with continued slow mixing to neutralize the polymer.

7. Continue slow mixing. When the batch temperature is below 40° C. add component 10 (preservative) and mix until uniform.

TABLE 18

| | | | Amount | |
|---|---|---|---|---|
| | | Component | (wt. %) | Function |
| A | 1 | PEG-20 Methyl Glucose | 1.80 | Emulsifier |
| | | Sequistearate | | |
| | 2 | DI Water | 70.14 | Diluent |
| B | 3 | Titanium Dioxide/Glycrerin/ | 10.00 | Pigment |
| | | Water (50%) | | |
| C | 4 | Isopropyl Isostearate | 5.00 | Emollient |
| | 5 | Diisopropyl Sebacate | 7.00 | Emollient |
| | 6 | Cetearyl Alcohol | 2.00 | Emulsifier |
| | 7 | Methyl Glucose Sequistearate | 1.20 | Emulsifier |
| D | 8 | Polymer of Ex. 11 (100% Active) | 0.7 | Rheology |
| | | | (active | Modifier |
| | | | wt. %) | |
| | 9 | Triethanolamine (99%) | 0.33 | Neutralizer |
| | 10 | DMDM Hydantoin (and) Idopropyl | 0.3 | Antimicrobial |
| | | Butylcarbamate | | |

Properties

| | |
|---|---|
| Appearance | Creamy Emulsion |
| Texture | Smooth Gel, Good |
| | Spreadability |
| pH | 6.9 |
| Viscosity (mPa · s), spindle no. 7 | 39400 |
| Suspension/Emulsion Stability | Pass |
| (3 months at 45° C.) | |

### Example 42 (Thickened Sulfate-Free Surfactant System)

Sulfate-free surfactant blends were formulated with 2.5 wt. % (100% active material) of HASE test polymer, 0.05 wt. % Kathon™ CG preservative and the surfactant blends forth in Table 19.

Procedure:

1. Disperse test polymer in 40 g of DI water and mix until uniform.

2a. Add each individual component of the formulated surfactant blend to the mixture obtained in step 1 in the following order: disodium laureth sulfosuccinate, sodium lauryl sarcosinate and CAPB and mix until uniform.

2b. Add the commercial surfactant blend to the mixture obtained in step 1.

3. Add the preservative to the mixtures obtained in step 2a and step 2b and mix until uniform.

4 Adjust the pH of the surfactant/polymer blends obtained in step 3 with 18% NaOH or 50% citric acid to the target pH value.

5. Add DI water (q.s. to 100) to achieve the desired target polymer concentration.

The polymers of the disclosed technology imparted good rheology properties, clarity and the ability to stably suspend particulate materials for at least one week at elevated temperature in sulfate-free surfactant systems.

TABLE 19

| | | pH = 6.5 | | | pH = 5.5 | | |
|---|---|---|---|---|---|---|---|
| | | | | Bead | | | Bead |
| | | | | Suspension | | | Suspension |
| | | | | Stability | | | Stability |
| | Polymer | Viscosity | Clarity | (1 wk. at | Viscosity | Clarity | (1 wk. at |
| Surfactant Chassis | Ex. No. | (mPa · s) | (NTU) | 45° C.) | (mPa · s) | (NTU) | 45° C.) |
| Disodium Laureth | 11 | 3632 | 7.2 | Pass | 8004 | 26.8 | Pass |
| Sulfosuccinate/CAPB/Sodium | 33 | 5575 | 18.8 | Pass | 18420 | 53.3 | Pass |
| Lauryl Sarcosinate¹ | 34 | 6392 | 12.8 | Pass | 18760 | 52.0 | Pass |
| (8/4/3 wt. % active) | | | | | | | |
| Disodium Laureth | 7 | 27460 | 7.4 | Partial | 11100 | 53.9 | Pass |
| Sulfosuccinate (and) Sodium | | | | Pass³ | | | |
| Cocoyl Isethionate (and) | 11 | 5537 | 4.7 | Pass | 6463 | 27.4 | Pass |
| Cocamidopropyl Betaine² | | | | | | | |
| (15 wt. % active) | | | | | | | |

¹Prepared Surfactant blend
²Commercial Surfactant blend
³Only able to stabilize Type 2 and Type 3 beads

1. An emulsion polymer prepared from a polymerizable monomer composition comprising:
(A) from about 10 wt. % to about 75 wt. % of at least one acidic vinyl monomer, salts thereof, and mixtures thereof;
(B) from about 10 wt. % to about 90 wt. % of at least one nonionic vinyl monomer represented by the formulas:
CH₂═C(X)Z, (I)
CH₂═CH—OC(O)R (II)
wherein, in each of formulas (I) and (II), X is H or methyl; and Z is —C(O)OR¹, —C(O)NH₂, —C(O)NHR¹, —C(O)N(R¹)₂, —C₆H₄R¹, —C₆H₄OR¹, —C₆H₄C₁, —CN, —NHC(O)CH₃, —NHC(O)H, N-(2-pyrrolidonyl), N-caprolactamyl, —C(O)NHC(CH₃)₃, —C(O)NHCH₂CH₂—N-ethyleneurea, —SiR₃, —C(O)O(CH₂)ₓSiR₃, —C(O)NH(CH₂)ₓSiR₃, or —(CH₂)ₓSiR₃; x is an integer ranging from about 1 to about 6; each R is independently C₁-C₁₈ alkyl; each R¹ is independently C₁-C₃₀ alkyl, hydroxy-substituted C₂-C₃₀ alkyl, or halogen-substituted C₁-C₃₀ alkyl;
(C) from about 0.1 wt. % to about 25 wt. % and from about 0.5 wt. % to about 15 wt. % of at least one associative monomer represented by the formula: 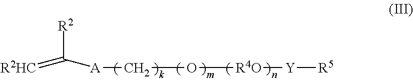
wherein, each R² is independently H, methyl, —C(O)OH, or —C(O)OR³; R³ is C₁-C₃₀ alkyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent aryl; E is H or methyl; z is 0 or 1; k is an integer in the range of 0 to about 30, and m is 0 or 1, with the proviso that when k is 0,
m is 0, and when k is in the range of 1 to about 30, m is 1; (R⁴-0)ₙ is a polyoxyalkylene, which is a homopolymer, a random copolymer, or a block copolymer of C₂-C₄ oxyalkylene units, wherein R⁴ is C₂H₄, C₃H₆, C₄H₈, and n is an integer in the range of about 2 to about 250, Y is —R⁴O—, —R⁴NH—, —C(O)—, —C(O)NH—, —R⁴NHC(O)NH—, or —C(O)NHC(O)—; and R⁵ is a substituted or unsubstituted alkyl selected from a C₈-C₄₀ linear alkyl, a C₈-C₄₀ branched alkyl, a C₈-C₄₀ carbocyclic alkyl, a C₂-C₄₀ alkyl-substituted phenyl, and an aryl-substituted C₂-C₄₀ alkyl; wherein the R⁵ alkyl group optionally contains one or more substituents selected from a hydroxyl group, an alkoxyl group, and a halogen group;
(D) from about 0.01 to about 20 wt. % (based on the wt. of total monounsaturated monomers) of a polyunsaturated amphiphilic macromonomer;
(E) from about 0 or 0.1 wt. % to about 25 wt. % of at least one semihydrophobic monomer represented by the formulas: 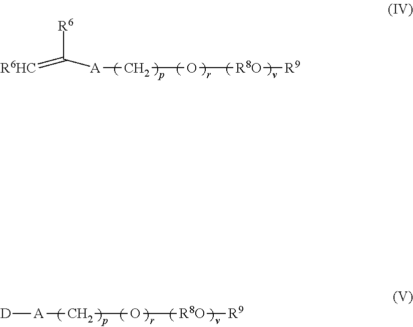
wherein, in each of formulas (IV) and (V), each R⁶ is independently H, C₁-C₃₀ alkyl, —C(O)OH, or —C(O)OR⁷; R⁷ is C₁-C₃₀ alkyl; A is —CH₂C(O)O—, —C(O)O—, —O—, —CH₂O—, —NHC(O)NH—, —C(O)NH—, —Ar—(CE₂)_{z}-NHC(O)O—, —Ar—(CE₂)_{z}-NHC(O)NH—, or —CH₂CH₂NHC(O)—; Ar is a divalent aryl; E is H or methyl; z is 0 or 1; p is an integer in the range of 0 to about 30, and r is 0 or 1, with the proviso that when p is 0, r is 0, and when p is in the range of 1 to about 30, r is 1; (R⁸—O)ᵥ is a polyoxyalkylene, which is a homopolymer, a random copolymer or a block copolymer of C₂-C₄ oxyalkylene units, wherein R⁸ is C₂H₄, C₃H₆, C₄H₈, and v is an integer in the range of from about 2 to about 250; R⁹ is H or C₁-C₄ alkyl; and D is a C₈-C₃₀ unsaturated alkyl or a carboxy-substituted C₈-C₃₀ unsaturated alkyl; optionally
(F) from about 0 or 0.1 wt. % to about 3 wt. % (based on the wt. of total monounsaturated monomers) of at least one polyunsaturated crosslinking monomer; and
wherein the sum of monomer components (A)-(F) totals 100 wt. %.

2. An emulsion polymer of claim 1 wherein said amphiphilic macromonomer (D) contains at least two polymerizable unsaturated groups.

3. An emulsion polymer of claim 1 wherein said amphiphilic macromonomer (D) contains at least two allyl groups.

4. An emulsion polymer of claim 1 wherein said amphiphilic monomer (D) is represented by the formula: 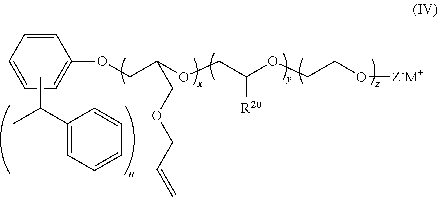
where R²⁰ is CH₃, CH₂CH₃, C₆H₅, or C₁₄H₂₉; n is 1, 2, or 3; x is 2-10, y is 0-200, z is 4-200, Z can be either SO₃⁻ or PO₃²⁻, and M⁺ is Na⁺, K⁺, NH₄⁺, or an alkanolamine such as, for example, monoethanolamine, diethanolamine, and triethanolamine.

5. An emulsion polymer of claim 1 wherein said amphiphilic monomer (D) is represented by the formula: 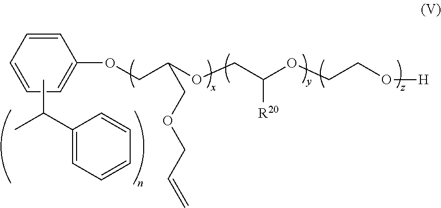
where R²⁰ is CH₃, CH₂CH₃, C₆H₅, or C₁₄H₂₉; n is 1, 2, 3; x is 2-10, y is 0-200, z is 4-200.

6. An emulsion polymer of claim 1 wherein said amphiphilic monomer (D) is represented by the formula: 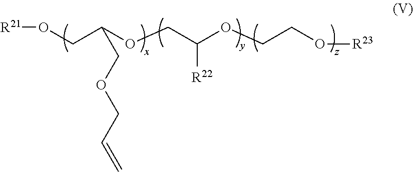
where R²¹ is a C₈-C₃₀ alkyl, alkaryl, alkenyl, or cycloalkyl group; R²² is CH₃, CH₂CH₃, C₆H₅, or C₁₄H₂₉; x is 2-100, y is 0-200; and R²³ is H or Z⁻M⁺, where Z can be SO₃⁻ or PO₃²⁻, and M⁺ is a salt forming cation.

7. An emulsion polymer of claim 6 wherein said salt forming cation M⁺ is selected from Na, K, and NH₄, or an alkanolamine.

8. An emulsion polymer of claim 1 wherein said monomer composition further comprises at least one polyunsaturated crosslinking monomer.

9. An emulsion polymer of claim 8 wherein said crosslinking monomer (F) is an acrylate ester of a polyol having at least two acrylate ester groups, a methacrylate ester of a polyol having at least two methacrylate ester groups, and mixtures thereof.

10. An emulsion polymer of claim 1 wherein said acidic vinyl monomer (A) is selected from acrylic acid, methacrylic acid, styrenesulfonic acid, 2-acrylamido-2-methylpropane sulfonic acid; and salts thereof; and mixtures thereof.

11. An emulsion polymer of claim 10 wherein said salt is selected from an alkali metal salt, an alkaline earth metal salt, an ammonium salt, an alkyl-substituted ammonium salt, and mixtures thereof.

12. An emulsion polymer of claim 1 wherein said nonionic vinyl monomer (B) is selected from a C₁-C₈ alkyl ester of (meth)acrylic acid, a hydroxy-substituted C₁-C₈ alkyl ester of (meth)acrylic acid, a vinyl C₂-C₁₀ alkanoate, N-vinyl pyrrolidone, and mixtures thereof.

13. An emulsion polymer of claim 1 wherein said nonionic vinyl monomer (B) is selected from ethyl acrylate, butyl acrylate, hydroxyethyl methacrylate, vinyl acetate, vinyl neodecanoate, N-vinyl pyrrolidone, and mixtures thereof.

14. An emulsion polymer of claim 1 wherein said at least one associative monomer (C) is selected from a monomer represented by the formula: 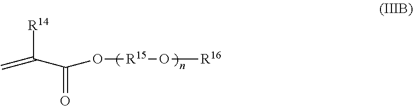
wherein R¹⁴ is hydrogen or methyl; R¹⁵ is a divalent alkylene moiety independently selected from C₂H₄, C₃H₆, and C₄H₈, and n represents an integer ranging from about 2 to about 150, and from about 15 to about 30 in a still further aspect, (R¹⁵—O) can be arranged in a random or a block configuration; R¹⁶ is a substituted or unsubstituted alkyl selected from a C₈-C₃₀ linear alkyl, a C₈-C₃₀ branched alkyl, an alkyl substituted and unsubstituted C₇-C₃₀ carbocyclic alkyl, a C₂-C₃₀ alkyl-substituted phenyl, and an aryl-substituted C₂-C₃₀ alkyl.

15. An emulsion polymer of claim 1 wherein said at least one associative monomer (C) is selected from lauryl polyethoxylated (meth)acrylate, cetyl polyethoxylated (meth)acrylate, cetearyl polyethoxylated (meth)acrylate, stearyl polyethoxylated (meth)acrylate, arachidyl polyethoxylated (meth)acrylate, behenyl polyethoxylated (meth)acrylate, lignoceryl polyethoxylated (meth)acrylate, cerotyl polyethoxylated (meth)acrylate, montanyl polyethoxylated (meth)acrylate, melissyl polyethoxylated (meth)acrylate, where the polyethoxylated portion of the monomer contains about 2 to about 60 ethylene oxide units.

16. An emulsion polymer of claim 1 wherein said at least one semihydrophobic monomer (E) is selected from a monomer represented by the formulas:
CH₂═C(R²⁵)C(O)O—(C₂H₄O)ₐ(C₃H₆O)_{b}—H IXA
CH₂═C(R²⁵)C(O)O—(C₂H₄O)ₐ(C₃H₆O)_{b}—CH₃ IXB
wherein R²⁵ is hydrogen or methyl, and “a” is an integer ranging from 0 or 2 to about 120 and “b” is an integer ranging from about 0 or 2 to about 120, subject to the proviso that “a” and “b” cannot be 0 at the same time.

17. An emulsion polymer of claim 1 wherein said at least one semihydrophobic monomer (E) is selected from methoxy polyethyleneglycol (meth)acrylate, polyethyleneglycol (meth)acrylate, and mixtures thereof where the polyethyleneglycol portion of the monomer contains about 2 to about 50 ethylene oxide units.

18. An emulsion polymer of claim 1 wherein said monomer composition further comprises from about 0.05 wt. % to about 10 wt. % of at least one chain transfer agent (G), based on the weight of said monomer composition, and wherein the sum of monomer components (A) through (F) and chain transfer agent (G) totals 100 wt. % of the monomer composition.

19. An emulsion polymer of claim 1 wherein said polymerizable monomer composition comprises:
(A) from about 30 wt. % to about 60 wt. % of at least one acidic vinyl monomer or a salt thereof selected from acrylic acid, methacrylic acid, 2-acrylamido-2-methylpropane sulfonic acid;
(B) from about 30 wt. % to about 60 wt. % of at least one nonionic vinyl monomer selected from ethyl acrylate, butyl acrylate, hydroxyethyl methacrylate, vinyl acetate, vinyl neodecanoate, N-vinyl pyrrolidone, and mixtures thereof;
(C) from about 0.5 wt. % to about 15 wt. % of at least one associative monomer selected from cetearyl polyethoxylated methacrylate, stearyl polyethoxylated methacrylate, behenyl polyethoxylated methacrylate, lignoceryl polyethoxylated methacrylate, cerotyl polyethoxylated methacrylate, montanyl polyethoxylated methacrylate, and mixtures thereof, wherein the polyethoxylated portion of the monomer contains from about 5 to about 50 ethylene oxide units;
(D) from 0.5 wt. % to about 10 wt. % of at least one amphiphilic macromonomer represented by the formula: 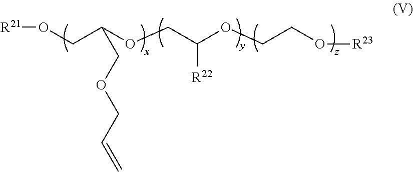
where R²¹ is a C₈-C₃₀ alkyl, alkaryl, alkenyl, or cycloalkyl group in one aspect, and a C₁₀-C₂₄ alkyl, aryl, alkylaryl, and aralkylaryl group in another aspect; R²² is CH₃, CH₂CH₃, C₆H₅, or C₁₄H₂₉; x is 2-100, z is 4-200; and R²³ is H or Z⁻M⁺, where Z can be SO₃⁻ or PO₃²⁻, and M⁺ is a salt forming cation.
(E) from about 0 or 0.5 wt. % to about 15 wt. % of at least one semihydrophobic monomer selected from methoxy polyethyleneglycol (meth)acrylate, polyethyleneglycol (meth)acrylate, and mixtures thereof where the polyethyleneglycol portion of the monomer contains about 2 to about 50 ethylene oxide units; and
(F) from about 0 or 0.1 wt. % to about 3 wt. % of at least one polyunsaturated crosslinking monomer.

20. An emulsion polymer of claim 19 wherein said at least one amphiphilic macromonomer (D) is represented by the formula: 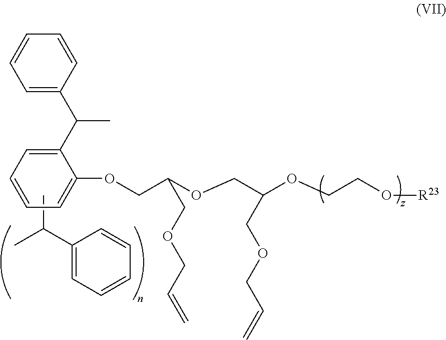
where n is 1 or 2; z is 4-40; and R²³ is H, SO₃⁻M⁺ or PO₃⁻M⁺, and M is a salt forming cation.

21. An emulsion polymer of claim 20 wherein said salt forming cation M⁺ is Na, K, and NH₄, or alkanolamine.

22. An emulsion polymer of claim 19 wherein said polymerizable monomer composition comprises:
(A) methacrylic acid;
(B) at least one nonionic monomer selected from ethyl acrylate, butyl acrylate, hydroxyethyl methacrylate, vinyl acetate, N-vinyl pyrrolidone, and mixtures thereof;
(C) at least one associative monomer selected from cetearyl polyethoxylated methacrylate, stearyl polyethoxylated methacrylate, behenyl polyethoxylated methacrylate, and mixtures thereof;
(D) at least one amphiphilic macromonomer represented by the formulas (VI) and (VII); optionally
(E) at least one semihydrophobic monomer selected from methoxy polyethyleneglycol (meth)acrylate, polyethyleneglycol (meth)acrylate, and mixtures thereof; and optionally
(F) at least one polyunsaturated crosslinking monomer.

23. An emulsion polymer of claim 22 wherein said polymerizable monomer composition comprises:
(A) methacrylic acid;
(B) a nonionic monomer selected from ethyl acrylate, butyl acrylate, and mixtures thereof;
(C) at least one associative monomer selected from cetearyl polyethoxylated methacrylate, stearyl polyethoxylated methacrylate, behenyl polyethoxylated methacrylate, and mixtures thereof;
(D) at least one amphiphilic macromonomer selected from a macromonomer represented by the formulas (VI) and (VII); optionally
(E) at least one semihydrophobic monomer selected from methoxy polyethyleneglycol (meth)acrylate, polyethyleneglycol (meth)acrylate, and mixtures thereof; and optionally
(F) at least one polyunsaturated crosslinking monomer.

24. An aqueous surfactant containing composition comprising:
(i) a surfactant selected from at least one anionic surfactant, at least one amphoteric surfactant, at least one nonionic surfactant, a least one cationic surfactant, and mixtures thereof;
(ii) at least one emulsion polymer selected from of claim 1; and
(iii) water.

25. An aqueous surfactant containing composition of claim 24 further comprising (iv) a neutralizing agent.

26. An aqueous surfactant containing composition of claim 24 comprising:
A) from about 5 wt. % to about 30 wt. % of surfactant component (i); and
B) from about 0.1 wt. % to about 10 wt. % of said emulsion polymer component (ii).

27. An aqueous surfactant containing composition of claim 25 wherein said neutralizing agent is present in an amount to attain a compositional pH ranging from about 3 to about 12.

28. An aqueous surfactant containing composition of claim 24 wherein said anionic surfactant is selected from alkali metal and ammonium salts of alkyl sulfates, alkyl ether sulfates, alkyl monoglyceryl ether sulfates, alkyl monoglyceride sulfates, alkyl monoglyceride sulfonates, alkyl sulfonates, alkylalkyl sulfonates, alkyl phosphates, alkyl sulfoacetates, alkyl sulfosuccinates, alkyl ether sulfosuccinates, alkyl amidosulfosuccinates, alkyl succinates, alkyl carboxylates, alkyl amidoether carboxylates, C₁₄-C₁₆ olefin sulfonates, acyl sarcosinates, acyl isethionates, acyl methyl isethionates, acyl N-methyl taurates, acyl glutamates, acyl lactylates, acyl glycinates, acyl alaninates, and mixtures thereof.

29. An aqueous surfactant containing composition of claim 24 wherein said anionic surfactant is selected from alkali metal or ammonium salts of saturated and unsaturated fatty acids containing from about 8 to about 22 carbon atoms, and mixtures thereof.

30. An aqueous surfactant containing composition of claim 24 wherein said amphoteric surfactant is selected from (mono- or di-)alkylamphoacetates, alkyl betaines, amidoalkyl betaines, amidoalkyl sultaines, and mixtures thereof.

31. An aqueous surfactant containing composition of claim 24 wherein said nonionic surfactant is selected from C₈-C₁₈ alkyl glucosides and polyglucosides, sucrose, glucose, sorbitol, sorbitan and polyglycerol esters of C₁₀-C₁₈ fatty acids.

32. An aqueous surfactant containing composition of claim 24 wherein said anionic surfactant is selected from an alkyl sulfate salt, an alkyl ether sulfate salt, a salt of a C₁₂ to C₂₂ fatty acid, and mixtures thereof.

33. An aqueous surfactant composition of claim 24 wherein said surfactant is selected from sodium and ammonium lauryl sulfate, sodium and ammonium lauryl ether sulfate, sodium C₁₄-C₁₆ alpha olefin sulfonate, and mixtures thereof.

34. An aqueous surfactant composition of claim 33 further comprising an amphoteric surfactant selected from lauryl betaine, cocamidopropyl betaine, cocamidopropyl hydroxysultaine, and mixtures thereof.

35. An aqueous surfactant containing composition of claim 33 wherein said sodium and ammonium lauryl ether sulfate salt contains 1 to 3 moles of ethylene oxide units.

36. An aqueous surfactant containing composition of claim 24 further comprising an insoluble material, a particulate material, or combinations thereof.

37. An aqueous surfactant containing composition of claim 36 wherein said particulate material is selected from mica, coated mica, pigments, exfoliants, anti-dandruff agents, clay, swellable clay, laponite, microsponges, cosmetic beads, cosmetic microcapsules, flakes, fragrance microcapsules, fragrance particles, and mixtures thereof.

38. An aqueous surfactant containing composition of claim 36 further comprising perfumes, fragrances, fragrance oils, and mixtures thereof.

39. An emulsion polymer of claim 1 wherein said at least one amphiphilic macromonomer (D) is represented by the formula:

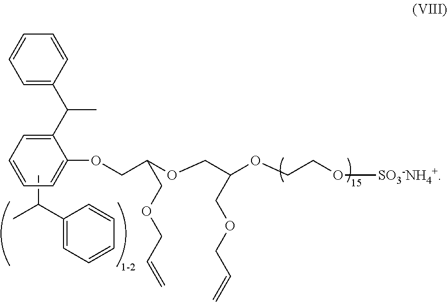

40. An emulsion polymer of claim 39 wherein said monomer composition is devoid of a conventional polyunsaturated crosslinking monomer.
